(19) United States
(12) Patent Application Publication
(10) Pub. No.: US 2021/0251988 A1
(43) Pub. Date: Aug. 19, 2021

(54) METHODS OF TREATING DISORDERS

(71) Applicant: Foghorn Therapeutics Inc., Cambridge, MA (US)

(72) Inventors: Qianhe ZHOU, Winchester, MA (US); Michael BOCKER, Cambridge, MA (US); Ho Man CHAN, Carlisle, MA (US); Luis SOARES, Cambridge, MA (US)

(21) Appl. No.: 17/254,046

(22) PCT Filed: Jun. 20, 2019
(86) PCT No.: PCT/US2019/038296

§ 371 Date: Dec. 18, 2020

Related U.S. Application Data
(60) Provisional application No. 62/688,274, filed on Jun. 21, 2018.

(51) Int. Cl. A61K 31/496; A61K 31/704; A61K 31/675; A61K 38/46; C12N 15/113; A61P 35/00; A61K 47/54; A61K 45/06; A61K 31/454; A61K 31/427; A61K 31/472

(52) U.S. Cl. CPC A61K 31/496; A61K 31/704; A61K 31/675; A61K 38/465; C12N 15/113; A61P 35/00; C12N 2310/141; A61K 45/06; A61K 31/454; A61K 31/427; A61K 31/472; C12N 2310/531; C12N 2310/14; A61K 47/54

(57) ABSTRACT

The present invention relates to methods and compositions for the treatment of BAF-related disorders such as cancers and viral infections.

### SEQUENCE LISTING

The instant application contains a Sequence Listing which has been submitted electronically in ASCII format and is hereby incorporated by reference in its entirety. Said ASCII copy, created on Jun. 20, 2019, is named 51121-023W02_Sequence_Listing_6.20.2019_ST25 and is 180,048 bytes in size.

### BACKGROUND

Disorders can be affected by the BAF complex. BICRA is a component of the BAF complex. The present invention relates to useful methods and compositions for the treatment of BAF-related disorders, such as cancer and infection.

### SUMMARY

BRD4 Interacting Chromatin Remodeling Complex Associated protein (BICRA) is a protein encoded by the BICRA gene on chromosome 19. BICRA is a component of the BAF (BRG1- or BRM-associated factors) complex, a SWI/SNF ATPase chromatin remodeling complex. BICRA is present in several SWI/SNF ATPase chromatin remodeling complexes and is upregulated in multiple cancer cell lines. Accordingly, agents which reduce the levels and/or activity of BICRA may provide new methods for the treatment of disease and disorders, such as cancer. Depleting BICRA in cells may result in the depletion of the SS18-SSX fusion protein in those cells. The SS18-SSX fusion protein has been detected in more than 95% of synovial sarcoma tumors and is often the only cytogenetic abnormality in synovial sarcoma. Thus, agents that degrade BICRA, e.g., antibodies, enzymes, polynucleotides, and compounds, may be useful in the treatment of cancers related to BICRA or SS18-SSX expression such as soft tissue sarcomas, e.g., synovial sarcoma.

The present disclosure features useful methods to treat cancer, e.g., in a subject in need thereof. In some embodiments, the methods described herein are useful in the treatment of disorders associated with BICRA expression, e.g., soft tissue sarcomas, e.g., adult soft tissue sarcomas. In some embodiments, the methods described herein are useful in the treatment of disorders associated with SS18-SSX fusion protein.

In one aspect, the invention features a method of treating soft tissue sarcoma (e.g., adult soft tissue sarcoma) in a subject in need thereof, the method including administering to the subject an effective amount of an agent that reduces the level and/or activity of BICRA in the sarcoma.

In another aspect, the invention features a method of treating soft tissue sarcoma (e.g., adult soft tissue sarcoma) in a subject in need thereof, the method including administering to the subject an effective amount of an agent that reduces the level and/or activity of a BAF complex (e.g., GBAF) in the sarcoma.

In another aspect, the invention features a method of reducing tumor growth of a (soft tissue sarcoma (e.g., an adult soft tissue sarcoma) in a subject in need thereof, the method including administering to the subject an effective amount of an agent that reduces the level and/or activity of BICRA in the tumor.

In another aspect, the invention features a method of inducing apoptosis in a soft tissue sarcoma (e.g., an adult soft tissue sarcoma) cell, the method including contacting the cell with an effective amount of an agent that reduces the level and/or activity of BICRA in the cell.

In another aspect, the invention features a method of reducing the level of BICRA in a soft tissue sarcoma (e.g., an adult soft tissue sarcoma) cell, the method including contacting the cell with an effective amount of an agent that reduces the level and/or activity of BICRA in the cell.

In some embodiments of any of the above aspects, the soft tissue sarcoma (e.g., adult soft tissue sarcoma) cell is in a subject. In some embodiments, the subject or cell has been identified as expressing SS18-SSX fusion protein or BICRA fusion protein.

In another aspect, the invention features a method of modulating the level of an SS18-SSX fusion protein, SS18 wild-type protein, or SSX wild-type protein in a cell or subject, the method including contacting the cell with an effective amount of an agent that reduces the level and/or activity of BICRA in a cell or subject. In some embodiments, the cell is in a subject.

In another aspect, the invention features a method of treating a disorder related to an SS18-SSX fusion protein, SS18 wild-type protein, or SSX wild-type protein in a subject in need thereof, the method including administering to the subject an effective amount of an agent that reduces the level and/or activity of BICRA in an SS18-SSX fusion protein-expressing cell in the subject.

In some embodiments of any of the above aspects, the effective amount of the agent reduces the level and/or activity of BICRA by at least 5% (e.g., 6%, 7%, 8%, 8%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the agent that reduces the level and/or activity of BICRA by at least 50% (e.g., 55%, 60%, 65%, 70%, 75%, 80%, 85%, 90%, or 95%) as compared to a reference. In some embodiments, the effective amount of the agent that reduces the level and/or activity of BICRA by at least 90% (e.g., 91%, 92%, 93%, 94%, 95%, 96%, 97%, 98%, or 99%).

In some embodiments, the effective amount of the agent reduces the level and/or activity of BICRA by at least 5% (e.g., 6%, 7%, 8%, 8%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the agent that reduces the level and/or activity of BICRA by at least 5% (e.g., 6%, 7%, 8%, 8%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses SS18-SSX fusion protein and/or the cell or subject has been identified as expressing SS18-SSX fusion protein. In some embodiments, the disorder is synovial sarcoma or Ewing's sarcoma. In some embodiments, the disorder is synovial sarcoma.

In one aspect, the invention features a method of modulating the activity of a BAF complex in a cell or subject, the method including contacting the cell with an effective amount of an agent that reduces the level and/or activity of BICRA in the cell or subject.

In another aspect, the invention features a method of increasing the level of BAF47 in a cell or subject, the method including contacting the cell with an effective amount of an agent that reduces the level and/or activity of BICRA in the cell or subject.

In one aspect, the invention features a method of decreasing Wnt/β-catenin signaling in a cell or subject, the method including contacting the cell with an effective amount of an agent that reduces the level and/or activity of BICRA in the cell or subject.

In one aspect, the invention features a method treating a disorder related to BAF47 in a subject in need thereof, the method including administering to the subject an effective amount of an agent that reduces the level and/or activity of BICRA in the subject.

In some embodiments, the disorder related to BAF47 is a cancer or viral infection. In some embodiments, the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer.

In some embodiments, the viral infection is an infection with a virus of the Retroviridae family, Hepadnaviridae family, Flaviviridae family, Adenoviridae family, Herpesviridae family, Papillomaviridae family, Parvoviridae family, Polyomaviridae family, Paramyxoviridae family, or Togaviridae family.

In an aspect, the invention features a method for treating cancer in a subject in need thereof, the method including administering to the subject an effective amount of an agent that reduces the level and/or activity of BICRA in a cancer cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

In an aspect, the invention features a method of reducing tumor growth of a cancer in a subject in need thereof, the method including administering to the subject an effective amount of an agent that reduces the level and/or activity of BICRA in a tumor cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

In another aspect, the invention features a method of inducing apoptosis in a cancer cell, the method including contacting the cell with an effective amount of an agent that reduces the level and/or activity of BICRA in the cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

In another aspect, the invention features a method of reducing the level of BICRA in a cancer cell, the method including contacting the cell with an effective amount of an agent that reduces the level and/or activity of BICRA in the cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

In some embodiments of any of the foregoing aspects, the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer. In some embodiments, the cancer is non-small cell lung cancer, stomach cancer, breast cancer, malignant rhabdoid tumor, multiple myeloma, or atypical teratoid rhabdoid tumor.

In one aspect, the invention features a method of modulating the activity of a BICRA fusion protein in a cell or subject, the method including contacting the cell with an effective amount of an agent that reduces the level and/or activity of BICRA in the cell or subject.

In another aspect, the invention features a method of modulating the level of a BICRA fusion protein in a cell or subject, the method including contacting the cell with an effective amount of an agent that reduces the level and/or activity of BICRA in the cell or subject. In some embodiments, the cell is in a subject.

In another aspect, the invention features a method of treating a disorder related to a BICRA fusion protein in a subject in need thereof, the method including administering to the subject an effective amount of an agent that reduces the level and/or activity of BICRA in a BICRA fusion protein-expressing cell.

In some embodiments of any of the above aspects, the subject has cancer. In some embodiments, the cancer expresses a BICRA fusion protein and/or the cell or subject has been identified as expressing a BICRA fusion protein. In some embodiments, the method further includes administering to the subject or contacting the cell with an anticancer therapy. In some embodiments, the anticancer therapy is a chemotherapeutic or cytotoxic agent or radiotherapy. In some embodiments, the chemotherapeutic or cytotoxic agent is doxorubicin or ifosfamide. In some embodiments, the anticancer therapy and the agent that reduces the level and/or activity of BICRA in a cell are administered within 28 days of each other and each in an amount that together are effective to treat the subject. In some embodiments, the subject or cancer has been identified as having an elevated level of an SS18-SSX fusion protein or a BICRA fusion protein as compared to a reference. In some embodiments, the subject or cancer has been identified as having a decreased level of SS18 wild-type protein or SSX wild-type protein as compared to a reference.

In one aspect, the invention features a method of treating a viral infection, the method including administering to the subject an effective amount of an agent that reduces the level and/or activity of BICRA in a cell of the subject.

In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma. In some embodiments, the viral infection is an infection with a virus of the Retroviridae family, Hepadnaviridae family, Flaviviridae family, Adenoviridae family, Herpesviridae family, Papillomaviridae family, Parvoviridae family, Polyomaviridae family, Paramyxoviridae family, or Togaviridae family.

In some embodiments of any of the above aspects, the agent that reduces the level and/or activity of BICRA in a cell is a small molecule compound, an antibody, an enzyme, and/or a polynucleotide. In some embodiments, the agent that reduces the level and/or activity of BICRA in a cell is an enzyme. In some embodiments, the enzyme is a clustered regularly interspaced short palindromic repeats (CRISPR)-associated protein, a zinc finger nuclease (ZFN), a transcription activator-like effector nuclease (TALEN), or a meganuclease. In some embodiments, the CRISPR-associated protein is CRISPR-associated protein 9 (Cas9).

In some embodiments of any of the above aspects, the agent that reduces the level and/or activity of BICRA in a cell is a polynucleotide. In some embodiments, the polynucleotide is an antisense nucleic acid, a short interfering RNA (siRNA), a short hairpin RNA (shRNA), a CRISPR/Cas 9 nucleotide (e.g., a guide RNA (gRNA)), or a ribozyme. In some embodiments, the polynucleotide has a sequence having at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 3-124. In some embodiments, the polynucleotide comprises a sequence having at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 3-68.

In some embodiments of any of the above aspects, the agent that reduces the level and/or activity of BICRA in a cell is a small molecule compound, or a pharmaceutically acceptable salt thereof.

In some embodiments, the small molecule compound, or a pharmaceutically acceptable salt thereof is a degrader. In some embodiments, the degrader has the structure of Formula I:

A-L-B Formula I

wherein A is a BICRA binding moiety; L is a linker; and B is a degradation moiety, or a pharmaceutically acceptable salt thereof. In some embodiments, the degradation moiety is a ubiquitin ligase moiety. In some embodiments, the ubiquitin ligase binding moiety includes Cereblon ligands, IAP (Inhibitors of Apoptosis) ligands, mouse double minute 2 homolog (MDM2), hydrophobic tag, or von Hippel-Lindau ligands, or derivatives or analogs thereof.

In some embodiments, the hydrophobic tag includes a diphenylmethane, adamantine, or tri-Boc arginine, i.e., the hydrophobic tag includes the structure:

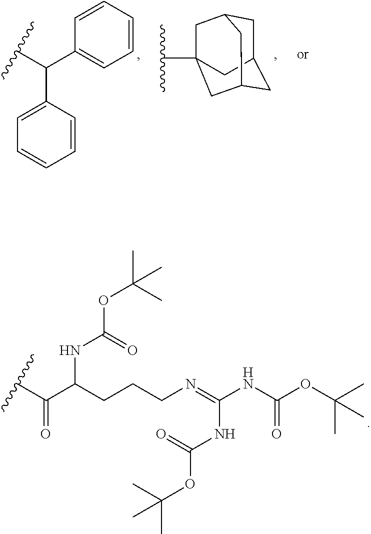

In some embodiments, the ubiquitin ligase binding moiety includes the structure of Formula A:

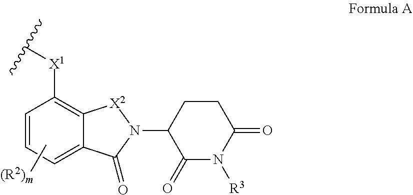

wherein X¹ is CH2, O, S, or NR¹, wherein R¹ is H, optionally substituted C1-C6 alkyl, or optionally substituted C1-C6 heteroalkyl; X² is C═O, CH₂, or

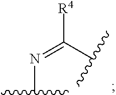

R³ and R⁴ are, independently, H, optionally substituted C1-C6 alkyl, or optionally substituted C1-C6 heteroalkyl; m is 0, 1, 2, 3, or 4; and each R² is, independently, halogen, optionally substituted C1-C6 alkyl, optionally substituted C1-C6 heteroalkyl, optionally substituted C3-C10 carbocyclyl, optionally substituted C2-C9 heterocyclyl, optionally substituted C6-C10 aryl, optionally substituted C2-C9 heteroaryl, optionally substituted C2-C6 alkenyl, optionally substituted C2-C6 heteroalkenyl, hydroxy, thiol, or optionally substituted amino, or a pharmaceutically acceptable salt thereof.

In some embodiments, the ubiquitin ligase binding moiety includes the structure:

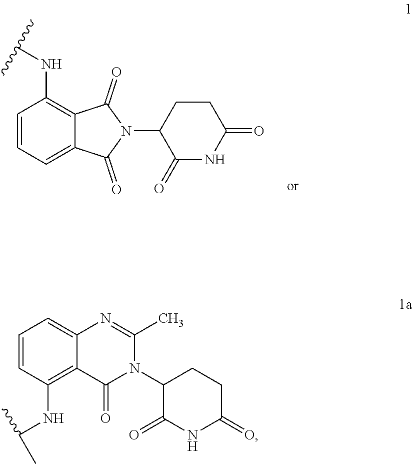

or is a derivative or an analog thereof, or a pharmaceutically acceptable salt thereof.

In some embodiments, the ubiquitin ligase binding moiety includes the structure of Formula B:

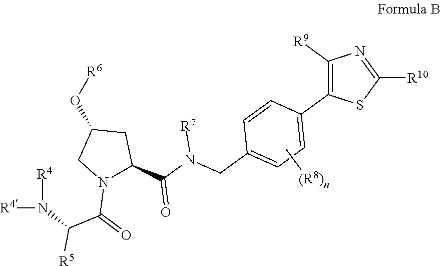

wherein each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; n is 0, 1, 2, 3, or 4; each R⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, wherein R^{4′} or R⁵ comprises a bond to the linker, or a pharmaceutically acceptable salt thereof.

In some embodiments, the ubiquitin ligase binding moiety includes the structure:

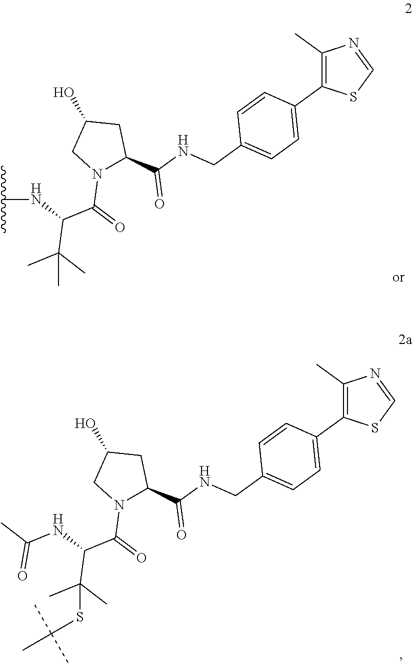

or is a derivative or analog thereof, or a pharmaceutically acceptable salt thereof.

In some embodiments, the ubiquitin ligase binding moiety includes the structure of Formula C:

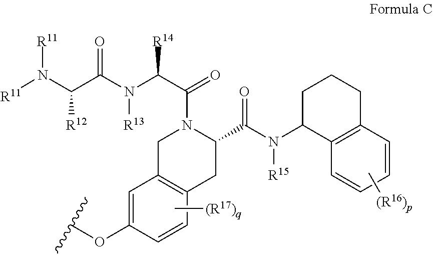

wherein each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

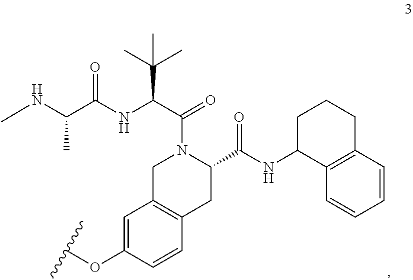 [SMILES: *Oc1ccc2c(c1)CN(C(=O)[C@@H](NC(=O)[C@H](C)NC)C(C)(C)C)[C@H](C(=O)NC1CCCc3ccccc31)C2]

or is a derivative or an analog thereof, or a pharmaceutically acceptable salt thereof.

In some embodiments, the ubiquitin ligase binding moiety includes the structure of Formula D:

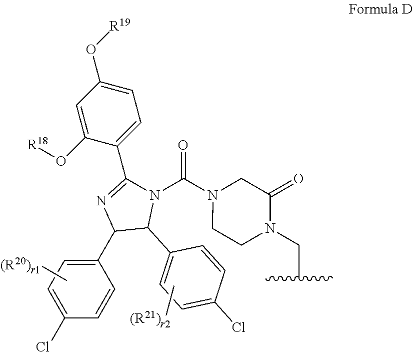

wherein each R¹⁸ and R¹⁹ is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the ubiquitin ligase binding moiety includes the structure:

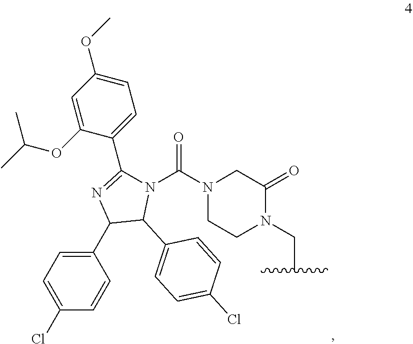 [SMILES: *CN1CCN(C(=O)N2C(c3ccc(OC)cc3OC(C)C)=NC(c3ccc(Cl)cc3)C2c2ccc(Cl)cc2)CC1=O]

or is a derivative or an analog thereof, or a pharmaceutically acceptable salt thereof.

In some embodiments, the linker has the structure of Formula II:

A¹-(B¹)_{f}—(C¹)_{g}—(B²)ₕ-(D)-(B³)ᵢ—(C²)ⱼ—(B⁴)ₖ-A² Formula II

wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, D is optionally substituted C₂-C₁₀ polyethylene glycol. In some embodiments, C¹ and C² are each, independently, a carbonyl or sulfonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen or optionally substituted C₁₋₄ alkyl. In some embodiments, B¹, B², B³, and B⁴ each, independently, is selected from optionally substituted C₁-C₂ alkyl or optionally substituted C₁-C₃ heteroalkyl. In some embodiments, j is 0. In some embodiments, k is 0. In some embodiments, j and k are each, independently, 0. In some embodiments, f, g, h, and i are each, independently, 1.

In some embodiments, the linker of Formula II has the structure of Formula IIa:

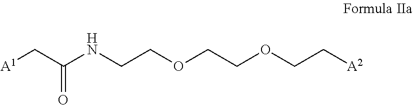

wherein A¹ is a bond between the linker and A, and A² is a bond between B and the linker.

In some embodiments, D is optionally substituted C₁₋₁₀ alkyl. In some embodiments, C¹ and C² are each, independently, a carbonyl. In some embodiments, B¹, B², B³, and B⁴ each, independently, is selected from optionally substituted C₁-C₂ alkyl, optionally substituted C₁-C₃ heteroalkyl, O, S, S(O)₂, and NR^{N}, wherein R^{N} is hydrogen or optionally substituted C₁₋₄ alkyl. In some embodiments, B¹, B², B³, and B⁴ each, independently, is selected from optionally substituted C₁-C₂ alkyl, O, S, S(O)₂, and NR^{N}, wherein R^{N} is hydrogen or optionally substituted C₁₋₄ alkyl. In some embodiments, B¹ and B⁴ each, independently, is optionally substituted C₁-C₂ alkyl. In some embodiments, B¹ and B⁴ each, independently, is C₁ alkyl. In some embodiments, B² and B⁴ each, independently, is NR^{N}, wherein R^{N} is hydrogen or optionally substituted C₁₋₄ alkyl. In some embodiments, B² and B⁴ each, independently, is NH. In some embodiments, f, g, h, l, j, and k are each, independently, 1.

In some embodiments, the linker of Formula II has the structure of Formula Mb:

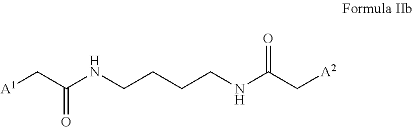

wherein A¹ is a bond between the linker and A, and A² is a bond between B and the linker.

In an aspect, the invention features a method of treating cancer in a subject, the method including: (a) determining the level of SS18-SSX fusion protein, SS18 wild-type protein, SSX wild-type protein, or a BICRA fusion protein in the subject; and (b) administering to the subject an effective amount of an agent that reduces the level and/or activity of BICRA in a cell or subject if the subject has an elevated level of SS18-SSX fusion protein or BICRA fusion protein or a decreased level of SS18 wild-type protein or SSX wild-type protein as compared to a reference. In a related aspect, the invention features a method of treating cancer in a subject determined to have an elevated level of SS18-SSX fusion protein, SS18 wild-type protein, SSX wild-type protein, or a BICRA fusion protein, the method including administering to the subject an effective amount of an agent that reduces the level and/or activity of BICRA in the cell or subject.

In some embodiments, the level of SS18-SSX fusion protein, SS18 wild-type protein, SSX wild-type protein, or a BICRA fusion protein in the subject is measured in one or more cancer cells. In some embodiments, the level of SS18-SSX fusion protein, SS18 wild-type protein, SSX wild-type protein, or a BICRA fusion protein in the subject is measured systemically.

In one aspect, the invention features a composition including an adult soft tissue sarcoma cell and an agent that reduces the level and/or activity of BICRA in a cell.

### Chemical Terms

For any of the following chemical definitions, a number following an atomic symbol indicates that total number of atoms of that element that are present in a particular chemical moiety. As will be understood, other atoms, such as hydrogen atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a hydrogen or an alkyl group that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O, or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds described herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds described herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “including” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “soft tissue sarcoma” refers to a sarcoma that develops in the soft tissues of the body (e.g., an adult soft tissue sarcoma). Adult soft tissue sarcoma refers to a sarcoma that develops typically in adolescent and adult subjects (e.g., subjects who are at least 10 years old, 11 years old, 12 years old, 13 years old, 14 years old, 15 years old, 16 years old, 17 years old, 18 years old, or 19 years old). Non-limiting examples of soft tissue sarcoma include, but are not limited to, synovial sarcoma, fibrosarcoma, malignant fibrous histiocytoma, dermatofibrosarcoma, liposarcoma, leiomyosarcoma, hemangiosarcoma, Kaposi's sarcoma, lymphangiosarcoma, malignant peripheral nerve sheath tumor/neurofibrosarcoma, extraskeletal chondrosarcoma, extraskeletal osteosarcoma, extraskeletal myxoid chondrosarcoma, and extraskeletal mesenchymal.

As used herein, the term “BAF complex” refers to the BRG1- or FIRBM-associated factors complex in a human cell.

As used herein, the terms “GBAF complex” and “GBAF” refer to a SWI/SNF ATPase chromatin remodeling complex in a human cell. GBAF complex subunits may include, but are not limited to, ACTB, ACTL6A, ACTL6B, BICRA, BICRAL, BRD9, SMARCA2, SMARCA4, SMARCC1, SMARCD1, SMARCD2, SMARCD3, and SS18.

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “BICRA” refers to BRD4 interacting chromatin remodeling complex associated protein (also called glioma tumor suppressor candidate region gene 1 protein or GLTSCR1), a component of the BAF (BRG1- or BRM-associated factors) complex, a SWI/SNF ATPase chromatin remodeling complex. BICRA is encoded by the BICRA gene. The nucleic acid sequence of an exemplary human BICRA is shown under NCBI Reference Sequence: NM_015711.3 or in SEQ ID NO: 1. The amino acid sequence of an exemplary protein encoded by human BICRA is shown under UniProt Accession No. Q9NZM4 or in SEQ ID NO: 2. The term “BICRA” also refers to natural variants of the wild-type BICRA protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type BICRA, an example of which is set forth in SEQ ID NO: 2.

As used herein, the term “degrader” refers to a small molecule compound including a degradation moiety, wherein the compound interacts with a protein (e.g., BICRA) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BICRA. In one example, the moiety binds to a protease or a ubiquitin ligase that metabolizes the protein, e.g., BICRA.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BICRA,” is meant decreasing the level of an activity related to BICRA, or a related downstream effect. A non-limiting example of inhibition of an activity of BICRA is decreasing the level of a BAF complex (e.g., GBAF) in a cell. The activity level of BICRA may be measured using any method known in the art. In some embodiments, an agent which reduces the activity of BICRA is a small molecule BICRA inhibitor. In some embodiments, an agent which reduces the activity of BICRA is a small molecule BICRA degrader.

By “reducing the level of BICRA,” is meant decreasing the level of BICRA in a cell or subject. The level of BICRA may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibitor” refers to any agent which reduces the level and/or activity of a protein (e.g., BICRA). Non-limiting examples of inhibitors include small molecule inhibitors, degraders, antibodies, enzymes, or polynucleotides (e.g., siRNA).

As used herein, the terms “effective amount,” “therapeutically effective amount,” and “a “sufficient amount” of an agent that reduces the level and/or activity of BICRA (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of BICRA sufficient to achieve a treatment response as compared to the response obtained without administration of the agent that reduces the level and/or activity of BICRA. The amount of a given agent that reduces the level and/or activity of BICRA described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of BICRA of the present disclosure is an amount which results in a beneficial or desired result in a subject as compared to a control. As defined herein, a therapeutically effective amount of an agent that reduces the level and/or activity of BICRA of the present disclosure may be readily determined by one of ordinary skill by routine methods known in the art. Dosage regimen may be adjusted to provide the optimum therapeutic response.

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., BICRA). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a BICRA polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared.

For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The details of one or more embodiments of the invention are set forth in the description below. Other features, objects, and advantages of the invention will be apparent from the description and from the claims.

### BRIEF DESCRIPTION OF THE DRAWINGS

FIG. 1 is a graph illustrating the effect of sgRNA targeting of the BICRA BAF complex subunit on synovial sarcoma cell growth. FIG. 1 corresponds to data obtained with SYO1 cell line. The Y-axis indicated the dropout ratio. The X-axis indicates the nucleotide position of the BICRA gene. The grey box indicates the range of the negative control sgRNAs in the screen. The SYO1 cell line carries SS18-SSX2 fusion protein. The linear protein sequence is shown with BICRA PFAM domains annotated from the PFAM database.

FIG. 2 is a graph illustrating the effect of sgRNA targeting of the BICRA BAF complex subunit on synovial sarcoma cell growth. FIG. 2 corresponds to data obtained with HS-SY-II cell line. The Y-axis indicated the dropout ratio. The X-axis indicates the nucleotide position of the BICRA gene. The grey box indicates the range of the negative control sgRNAs in the screen. The HS-SY-II cell line carries a SS18-SSX1 fusion protein. The linear protein sequence is shown with BICRA PFAM domains annotated from the PFAM database.

### DETAILED DESCRIPTION

The present inventors have found that depletion of BICRA in cancer cells inhibits cell growth and may result in the depletion of the SS18-SSX fusion protein and further inhibits the proliferation of the cancer cells.

Accordingly, the invention features methods and compositions useful for the inhibition of the activity of the SS18-SSX fusion proteins, e.g., for the treatment of cancer such as soft tissue sarcomas, e.g., adult soft tissue sarcomas. The invention further features methods and compositions useful for inhibition of the activity of the BICRA protein, e.g., for the treatment of cancer such as soft tissue sarcomas, e.g., in a subject in need thereof. Exemplary methods are described herein.

### BICRA-Reducing Agents

Agents described herein that reduce the level and/or activity of BICRA in a cell may be an antibody, a protein (such as an enzyme), a polynucleotide, or a small molecule compound. The agents reduce the level of an activity related to BICRA, or a related downstream effect, or reduce the level of BICRA in a cell or subject.

In some embodiments, the agent that reduces the level and/or activity of BICRA in a cell is an enzyme, a polynucleotide, or a small molecule compound such as a degrader or small molecule BICRA inhibitor.

Antibodies

The agent that reduces the level and/or activity of BICRA can be an antibody or antigen binding fragment thereof. For example, an agent that reduces the level and/or activity of BICRA described herein is an antibody that reduces or blocks the activity and/or function of BICRA through binding to BICRA. The making and use of therapeutic antibodies against a target antigen (e.g., BICRA) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Polynucleotides

In some embodiments, the agent that reduces the level and/or activity of BICRA is a polynucleotide. In some embodiments, the polynucleotide is an inhibitory RNA molecule, e.g., that acts by way of the RNA interference (RNAi) pathway. An inhibitory RNA molecule can decrease the expression level (e.g., protein level or mRNA level) of BICRA. For example, an inhibitory RNA molecule includes a short interfering RNA (siRNA), short hairpin RNA (shRNA), and/or a microRNA (miRNA) that targets full-length BICRA. A siRNA is a double-stranded RNA molecule that typically has a length of about 19-25 base pairs. A shRNA is a RNA molecule including a hairpin turn that decreases expression of target genes via RNAi. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. Degradation is caused by an enzymatic, RNA-induced silencing complex (RISC).

In some embodiments, the agent that reduces the level and/or activity of BICRA is an antisense nucleic acid. Antisense nucleic acids include antisense RNA (asRNA) and antisense DNA (asDNA) molecules, typically about 10 to 30 nucleotides in length, which recognize polynucleotide target sequences or sequence portions through hydrogen bonding interactions with the nucleotide bases of the target sequence (e.g., BICRA). The target sequences may be single- or double-stranded RNA, or single- or double-stranded DNA.

In embodiments, the polynucleotide decreases the level and/or activity of a negative regulator of function or a positive regulator of function. In other embodiments, the polynucleotide decreases the level and/or activity of an inhibitor of a positive regulator of function.

A polynucleotide of the invention can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The polynucleotides mentioned above, may also be provided in a specialized form such as liposomes, microspheres, or may be applied to gene therapy, or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. These moieties may be attached to the nucleic acid at the 3′ or 5′ ends and may also be attached through a base, sugar, or intramolecular nucleoside linkage. Other moieties may be capping groups specifically placed at the 3′ or 5′ ends of the nucleic acid to prevent degradation by nucleases such as exonuclease, RNase, etc. Such capping groups include hydroxyl protecting groups known in the art, including glycols such as polyethylene glycol and tetraethylene glycol. The inhibitory action of the polynucleotide can be examined using a cell-line or animal based gene expression system of the present invention in vivo and in vitro. In some embodiments, the polynucleotide decreases the level and/or activity or function of BICRA. In embodiments, the polynucleotide inhibits expression of BICRA. In other embodiments, the polynucleotide increases degradation of BICRA and/or decreases the stability (i.e., half-life) of BICRA. The polynucleotide can be chemically synthesized or transcribed in vitro.

Inhibitory polynucleotides can be designed by methods well known in the art. siRNA, miRNA, shRNA, and asRNA molecules with homology sufficient to provide sequence specificity required to uniquely degrade any RNA can be designed using programs known in the art, including, but not limited to, those maintained on websites for Thermo Fisher Scientific, the German Cancer Research Center, and The Ohio State University Wexner Medical Center. Systematic testing of several designed species for optimization of the inhibitory polynucleotide sequence can be routinely performed by those skilled in the art. Considerations when designing interfering polynucleotides include, but are not limited to, biophysical, thermodynamic, and structural considerations, base preferences at specific positions in the sense strand, and homology. The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010. Exemplary inhibitory polynucleotides, for use in the methods of the invention, are provided in Table 1, below. In some embodiments, the inhibitory polynucleotides have a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of an inhibitory polynucleotide in Table 1. In some embodiments, the inhibitory polynucleotides have a nucleic acid sequence with at least 70% sequence identity (e.g., 80%, 85%, 86%, 87%, 88%, 89%, 90%, 91%, 92%, 93%, 94%, 95%, 96%, 97%, 98%, 99%, 99.9% identity, or more) to the nucleic acid sequence of an inhibitory polynucleotide in Table 1.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

Gene Editing

In some embodiments, the agent that reduces the level and/or activity of BICRA is a component of a gene editing system. For example, the agent that reduces the level and/or activity of BICRA introduces an alteration (e.g., insertion, deletion (e.g., knockout), translocation, inversion, single point mutation, or other mutation) in BICRA. In some embodiments, the agent that reduces the level and/or activity of BICRA is a nuclease. Exemplary gene editing systems include the zinc finger nucleases (ZFNs), Transcription Activator-Like Effector-based Nucleases (TALENs), and the clustered regulatory interspaced short palindromic repeat (CRISPR) system. ZFNs, TALENs, and CRISPR-based methods are described, e.g., in Gaj et al., Trends Biotechnol. 31 (7):397-405 (2013).

CRISPR refers to a set of (or system including a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482(7385):331-338 (2012). For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that include a repeat sequence flanked by a spacer. The RNAs serve as guides to direct Cas proteins to silence specific DNA/RNA sequences, depending on the spacer sequence. See, e.g., Horvath et al., Science 327(5962):167-170 (2010); Makarova et al., Biology Direct 1:7 (2006); Pennisi, Science 341 (6148):833-836 (2013). In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a BICRA sequence.

In some embodiments, the agent that reduces the level and/or activity of BICRA includes a guide RNA (gRNA) for use in a CRISPR system for gene editing. Exemplary gRNAs, for use in the methods of the invention, are provided in Table 1, below. In embodiments, the agent that reduces the level and/or activity of BICRA includes a ZFN, or an mRNA encoding a ZFN, that targets (e.g., cleaves) a nucleic acid sequence (e.g., DNA sequence) of BICRA. In embodiments, the agent that reduces the level and/or activity of BICRA includes a TALEN, or an mRNA encoding a TALEN, that targets (e.g., cleaves) a nucleic acid sequence (e.g., DNA sequence) of BICRA.

For example, the gRNA can be used in a CRISPR system to engineer an alteration in a gene (e.g., BICRA). In other examples, the ZFN and/or TALEN can be used to engineer an alteration in a gene (e.g., BICRA). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) BICRA, e.g., the alteration is a negative regulator of function. In yet another example, the alteration corrects a defect (e.g., a mutation causing a defect), in BICRA. In certain embodiments, the CRISPR system is used to edit (e.g., to add or delete a base pair) a target gene, e.g., BICRA. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a target gene, e.g., BICRA, thereby blocking an RNA polymerase sterically.

In some embodiments, a CRISPR system can be generated to edit BICRA using technology described in, e.g., U.S. Publication No. 20140068797; Cong et al., Science 339(6121):819-823 (2013); Tsai, Nature Biotechnd, 32(6):569-576 (2014); and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding BICRA. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits BICRA. In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Bid. 17(1):5-15 (2016), incorporated herein by reference.

TABLE 1
Exemplary Inhibitory Polynucleotides

| SEQ | Type of | |
|---|---|---|
| ID | Inhibitory | |
| NO. | Polynucleotide | Nucleic Acid Sequence |
| 3 | CRISPR gRNA | GGAGGGCGCCCTGGTAGACA |
| 4 | CRISPR gRNA | ATATCGGCTCCTGCTCCTGG |
| 5 | CRISPR gRNA | TCCTGCTCCTGGAGGAGTCC |
| 6 | CRISPR gRNA | GGCGCCCTGGTAGACATGGT |
| 7 | CRISPR gRNA | TGCAGGGCGTCCTCAAAGGA |
| 8 | CRISPR gRNA | GCAGCTGCTGAAACGCACCC |
| 9 | CRISPR gRNA | GATCATTACCATCTCCGCTG |
| 10 | CRISPR gRNA | CCTGCCCTACCATGTCTACC |
| 11 | CRISPR gRNA | GAAGTCTAGGTCCACACTGG |
| 12 | CRISPR gRNA | CCTGGTAGACATGGTAGGGC |
| 13 | CRISPR gRNA | TCATTACCATCTCCGCTGAG |
| 14 | CRISPR gRNA | GGTAGGGCAGGAGGCGATGC |
| 15 | CRISPR gRNA | GCAGGGCGTCCTCAAAGGAG |
| 16 | CRISPR gRNA | TCAGGGACCAGGTGGAGGGT |
| 17 | CRISPR gRNA | TCTGCAGGGAGTCCTGAGTG |
| 18 | CRISPR gRNA | CTGCCCTACCATGTCTACCA |
| 19 | CRISPR gRNA | GTAGGGCAGGAGGCGATGCA |
| 20 | CRISPR gRNA | AGGCCATGCTCAATAAATAT |
| 21 | CRISPR gRNA | ACAGCTGGCCAAGGAGAAGC |
| 22 | CRISPR gRNA | ATGCAGGGCGTCCTCAAAGG |
| 23 | CRISPR gRNA | GCCTCCTCGGACCTTCCAGA |
| 24 | CRISPR gRNA | AGAAGTCATTGAGGGCCTGT |
| 25 | CRISPR gRNA | TCTCCTCCTGAATGAACATT |
| 26 | CRISPR gRNA | TTCTGGAGGATGATTCCGGA |
| 27 | CRISPR gRNA | GCAGGAAGGGCTGCACACTC |
| 28 | CRISPR gRNA | GGGGCCTGGTGAGGTAGTGA |
| 29 | CRISPR gRNA | CCTTGCTGGGCTGAAGCGTG |
| 30 | CRISPR gRNA | ATCATTACCATCTCCGCTGA |
| 31 | CRISPR gRNA | TTCCTTGCTGGGCTGAAGCG |
| 32 | CRISPR gRNA | GACGGCCTTCCCCTCCTTTG |
| 33 | CRISPR gRNA | GCTGGTGGCCTCGTCCACTT |
| 34 | CRISPR gRNA | AAGTGGACGAGGCCACCAGC |
| 35 | CRISPR gRNA | CAGCTGTTTATCCAAGGCAA |
| 36 | CRISPR gRNA | GGTAGACATGGTAGGGCAGG |
| 37 | CRISPR gRNA | GGAGCATTTGCACAAACACC |
| 38 | CRISPR gRNA | TTCAGGAGGTGGACGCTCAT |
| 39 | CRISPR gRNA | TCTAGGTCCACACTGGGGGC |
| 40 | CRISPR gRNA | GCCCCAGGACGATCTTCTCC |
| 41 | CRISPR gRNA | GACACACTCTGTGGCCGGGA |
| 42 | CRISPR gRNA | CTTGGCCAGGAGCTGGGAGG |
| 43 | CRISPR gRNA | GAGCTGTCCACCTGTGTGGG |
| 44 | CRISPR gRNA | CGGAAGAGGCTGCGATGGGG |
| 45 | CRISPR gRNA | GCGATGCAGGGCGTCCTCAA |
| 46 | CRISPR gRNA | GTTCAGGAGGTGGACGCTCA |
| 47 | CRISPR gRNA | TCCCCGCCGCCATGAACGTC |
| 48 | CRISPR gRNA | CTTGTTCTGGAGGATGATTC |
| 49 | CRISPR gRNA | GGCCTGGTGAGGTAGTGACG |
| 50 | CRISPR gRNA | GGGCCTCCCCGGGATTATCC |
| 51 | CRISPR gRNA | GTCCCCGTCACTACCTCACC |
| 52 | CRISPR gRNA | CTTGTAGTCGGGGTGCAGGA |
| 53 | CRISPR gRNA | CTCTGGGTTCAGGTGGTTGC |
| 54 | CRISPR gRNA | GCTGCCTGGGAAGAGGGCTT |
| 55 | CRISPR gRNA | CCTCCCCGGGATTATCCAGG |
| 56 | CRISPR gRNA | TCGAGAAGAGCCTTCGGCTG |
| 57 | CRISPR gRNA | GTTGTGACTGGAGGGTGGGT |
| 58 | CRISPR gRNA | GATGAGCTGTCCACCTGTGT |
| 59 | CRISPR gRNA | GCTGGAGGATGTCACAGGGC |
| 60 | CRISPR gRNA | TGCCGGATCACAAGCTTGGT |
| 61 | CRISPR gRNA | TCCCCCTCCAACCCTCCACC |
| 62 | CRISPR gRNA | AGTGGACGAGGAGTTTGAGA |
| 63 | CRISPR gRNA | GTAGTGACGGGGACGAAGAG |
| 64 | CRISPR gRNA | GGGTGCAAGGGTGGCTCTGA |
| 65 | CRISPR gRNA | GACCCCCTGGAGGACAGTGG |
| 66 | CRISPR gRNA | GAGCATTTGCACAAACACCA |
| 67 | CRISPR gRNA | GGGCCTGGTGAGGTAGTGAC |
| 68 | CRISPR gRNA | GGGCCTTGTTGACCACGTCC |
| 69 | CRISPR gRNA | GCCCAAAGTGCCTTCTATGA |
| 70 | CRISPR gRNA | CAACATCACGGAGCAGACGC |
| 71 | CRISPR gRNA | TGCCGGAAGCTTCTTGCACA |
| 72 | CRISPR gRNA | CCGTGATGTTGGCCTCTTGG |
| 73 | CRISPR gRNA | GCTCCGTGATGTTGGCCTCT |
| 74 | CRISPR gRNA | CAGCGTCTGCTCCGTGATGT |
| 75 | CRISPR gRNA | TGACCTCCTGGATAATCCCG |
| 76 | CRISPR gRNA | GGCCTTGTTGACCACGTCCT |
| 77 | CRISPR gRNA | CTGTGGCCACCACGCTCAAT |
| 78 | CRISPR gRNA | TGACGGGCTGGCCCACCACC |
| 79 | miRNA | CCACACAGCGGAGGGAGGCGGC |
| 80 | miRNA | GGGGAGAGCGAGAGCCCGGCUG |
| 81 | miRNA | CCUCCUUUCCGAGGGGCGUCGU |
| 82 | miRNA | UUCUUCAGCGGACUCAGUUUGC |
| 83 | miRNA | UGCACCGUCUCGACAGGCGCGG |
| 84 | miRNA | UUAUCCAACCGAAGGGUGGUCU |
| 85 | miRNA | UCUCUCACAGUCUUGUGCACAC |
| 86 | miRNA | UGUCGAUGCGCCUCUGCAGGUG |
| 87 | miRNA | UGCACACAGUGACACACACAGG |
| 88 | miRNA | UGUCAAGCAAGUCGGAUCCAUG |
| 89 | miRNA | UGCUCCAGCUUACAGGCUUCCU |
| 90 | miRNA | UUGUGCACCGGCUCGCUGAGCC |
| 91 | miRNA | UGUGCACAGUGACACACACACA |
| 92 | siRNA (guide | GGAGAATTCTGTACATTTA |
| | strand) | |
| 93 | siRNA (guide | GTATAACGATTTTTTTAAA |
| | strand) | |
| 94 | siRNA (guide | CTTTGAAATCTGAGCAAAA |
| | strand) | |
| 95 | siRNA (guide | CTGTAAGATAAATTTTTTT |
| | strand) | |
| 96 | siRNA (guide | CATTTAGAACTCTTGTAAA |
| | strand) | |
| 97 | siRNA (guide | GTGATGACCTCCTGGATAA |
| | strand) | |
| 98 | siRNA (guide | GCATCTTTGTCATCCAAAA |
| | strand) | |
| 99 | siRNA (guide | CCCAGGCCATGCTCAATAA |
| | strand) | |
| 100 | siRNA (guide | GGCCATGCTCAATAAATAT |
| | strand) | |
| 101 | siRNA (guide | CCTCAGCGGAGATGGTAAT |
| | strand) | |
| 102 | siRNA (guide | CCACCCTTGCCTTGGATAA |
| | strand) | |
| 103 | siRNA (guide | CACCCCTCGACTTTGAAAT |
| | strand) | |
| 104 | siRNA (guide | CCGCGCCAGATTTTGAAAT |
| | strand) | |
| 105 | siRNA (guide | CCTGTTTCCTGGAGCATTT |
| | strand) | |
| 106 | siRNA (guide | GGCCCAAAGTGCCTTCTAT |
| | strand) | |
| 107 | siRNA (guide | CCAGGACGTTGACCAGATA |
| | strand) | |
| 108 | siRNA (guide | CTGTATTTATTGTGTATAA |
| | strand) | |
| 109 | siRNA (guide | CCGGAATCATCCTCCAGAA |
| | strand) | |
| 110 | siRNA (guide | CCAGGCCATGCTCAATAAA |
| | strand) | |
| 111 | siRNA (guide | CAGGCCATGCTCAATAAAT |
| | strand) | |
| 112 | shRNA (loop | GAGGATGGGAGATGCTTACTATCAAGAGTAGTAAGCATCTCCCATCCTC |
| | bolded) | |
| 113 | shRNA (loop | GGATGGGAGATGCTTACTAGATCAAGAGTCTAGTAAGCATCTCCCATCC |
| | bolded) | |
| 114 | shRNA (loop | GATGCTTACTAGACGTGATTTTCAAGAGAAATCACGTCTAGTAAGCATC |
| | bolded) | |
| 115 | shRNA (loop | GGATCCGAGAAGCTTGACAGTTCAAGAGACTGTCAAGCTTCTCGGATCC |
| | bolded) | |
| 116 | shRNA (loop | GAAGCTTGACAGTGATGACCTTCAAGAGAGGTCATCACTGTCAAGCTTC |
| | bolded) | |
| 117 | shRNA (loop | GGCCCAAAGTGCCTTCTATGATCAAGAGTCATAGAAGGCACTTTGGGCC |
| | bolded) | |
| 118 | shRNA (loop | GCCCAAAGTGCCTTCTATGAATCAAGAGTTCATAGAAGGCACTTTGGGC |
| | bolded) | |
| 119 | shRNA (loop | GCAAACTGAGTGGCCTGAAGATCAAGAGTCTTCAGGCCACTCAGTTTGC |
| | bolded) | |
| 120 | shRNA (loop | GGTAATGATCGACCGAATGTTTCAAGAGAACATTCGGTCGATCATTACC |
| | bolded) | |
| 121 | shRNA (loop | GCCAGGACGTTGACCAGATAATCAAGAGTTATCTGGTCAACGTCCTGGC |
| | bolded) | |
| 122 | shRNA (loop | GTGCACAAGTGAGTGAGAGATTCAAGAGATCTCTCACTCACTTGTGCAC |
| | bolded) | |
| 123 | shRNA (loop | GCACAAGTGAGTGAGAGATTTTCAAGAGAAATCTCTCACTCACTTGTGC |
| | bolded) | |
| 124 | shRNA (loop | GCTAGTCTTCCCTCTGTTCTTTCAAGAGAAGAACAGAGGGAAGACTAGC |
| | bolded) | |

Small Molecule Compounds

In some embodiments of the invention, the agent that reduces the level and/or activity of BICRA in a cell is a small molecule compound. In some embodiments, the small molecule compound is a structure of Formula I:

A-L-B Formula I

wherein A is a BICRA binding moiety; L is a linker; and B is a degradation moiety.

In some embodiments, the degradation moiety has the structure of:

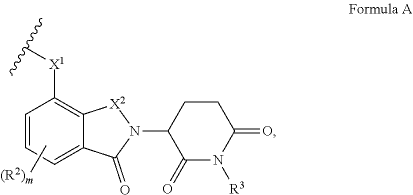

wherein X¹ is CH₂, O, S, or NR¹, wherein R¹ is H, optionally substituted C₁-C₆ alkyl, or optionally substituted C₁-C₆ heteroalkyl; X² is C═O, CH₂, or

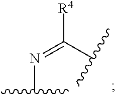

R³ and R⁴ are, independently, H, optionally substituted C₁-C₆ alkyl, or optionally substituted C₁-C₆ heteroalkyl; m is 0, 1, 2, 3, or 4; and each R² is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino,

or a pharmaceutically acceptable salt thereof;

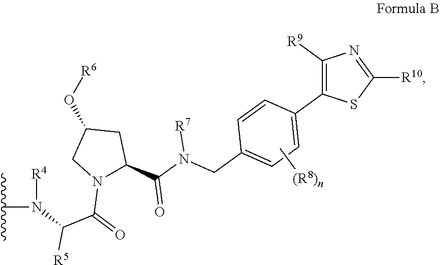

wherein each R⁴, R^{4′}, and R⁷ is, independently, H, optionally substituted C₁-C₆ alkyl, or optionally substituted C₁-C₆ heteroalkyl; R⁵ is optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R⁶ is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; n is 0, 1, 2, 3, or 4; each R⁸ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; and each R⁹ and R¹⁰ is, independently, H, halogen, optionally substituted C₁-C₆ alkyl, or optionally substituted C₆-C₁₀ aryl, wherein R^{4′} or R⁵ comprises a bond to the linker, or a pharmaceutically acceptable salt thereof;

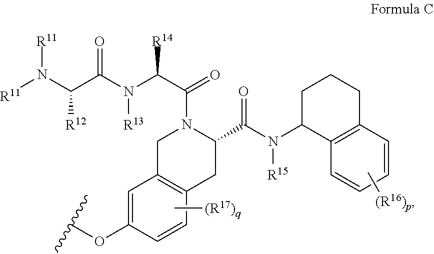

wherein each R¹¹, R¹³, and R¹⁵ is, independently, H, optionally substituted C₁-C₆ alkyl, or optionally substituted C₁-C₆ heteroalkyl; R¹² is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; R¹⁴ is optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; p is 0, 1, 2, 3, or 4; each R¹⁶ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; q is 0, 1, 2, 3, or 4; and each R¹⁷ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof; or

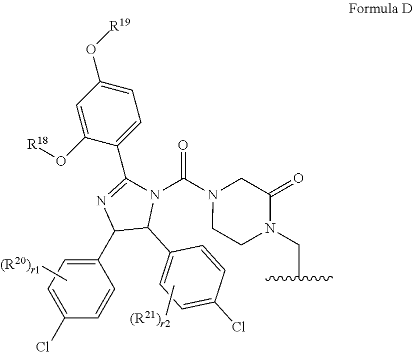

wherein each R¹⁸ and R¹⁹ is, independently, H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl; r1 is 0, 1, 2, 3, or 4; each R²⁰ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino; r2 is 0, 1, 2, 3, or 4; and each R²¹ is, independently, halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino, or a pharmaceutically acceptable salt thereof.

In some embodiments, the linker has the structure of Formula II:

A¹-(B¹)_{f}—(C¹)_{g}—(B²)ₕ-(D)-(B³)ᵢ—(C²)ⱼ—(B⁴)ₖ-A² Formula II

wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

Linkers include, but are not limited to, the structure of:

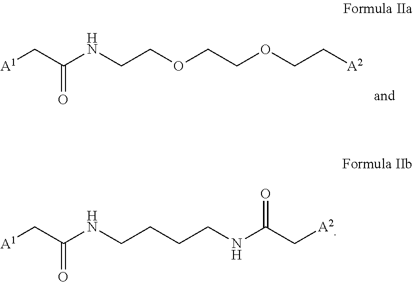

### Pharmaceutical Uses

The compounds described herein are useful in the methods of the invention and, while not bound by theory, are believed to exert their desirable effects through their ability to modulate the level, status, and/or activity of a BAF complex, e.g., by inhibiting the activity or level of the BRG and BRM proteins in a cell within the BAF complex in a mammal.

An aspect of the present invention relates to methods of treating disorders related to BRG and BRM proteins such as cancer in a subject in need thereof. In some embodiments, the compound is administered in an amount and for a time effective to result in one of (or more, e.g., two or more, three or more, four or more of): (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound described herein. An increase in average survival time of a population may also be measured, for example, by calculating for a population the average length of survival following completion of a first round of treatment with a pharmaceutically acceptable salt of a compound described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of a compound described herein. A decrease in the mortality rate of a population may also be measured, for example, by calculating for a population the average number of disease-related deaths per unit time following completion of a first round of treatment with a pharmaceutically acceptable salt of a compound described herein.

### Combination Therapies

A method of the invention can be used alone or in combination with an additional therapeutic agent, e.g., other agents that treat cancer or symptoms associated therewith, or in combination with other types of therapies to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-I-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The second agent may be a therapeutic agent which is a non-drug treatment. For example, the second therapeutic agent is radiation therapy, cryotherapy, hyperthermia, and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or fusion a protein such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In some embodiments, the anti-cancer therapy is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

Delivery of Anti-BICRA Agents

A variety of methods are available for the delivery of anti-BICRA agents to a subject including viral and non-viral methods.

Viral Delivery Methods

In some embodiments, the agent that reduces the level and/or activity of BICRA is delivered by a viral vector (e.g., a viral vector expressing an anti-BICRA agent). Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papillomavirus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Exemplary viral vectors include lentiviral vectors, AAVs, and retroviral vectors. Lentiviral vectors and AAVs can integrate into the genome without cell divisions, and both types have been tested in pre-clinical animal studies. Methods for preparation of AAVs are described in the art e.g., in U.S. Pat. Nos. 5,677,158, 6,309,634, and 6,683,058, each of which is incorporated herein by reference. Methods for preparation and in vivo administration of lentiviruses are described in US 20020037281 (incorporated herein by reference). Preferably, a lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding the fusion protein, an origin of second strand DNA synthesis and a 3′ lentiviral LTR.

Retroviruses are most commonly used in human clinical trials, as they carry 7-8 kb, and have the ability to infect cells and have their genetic material stably integrated into the host cell with high efficiency (see, e.g., WO 95/30761; WO 95/24929, each of which is incorporated herein by reference). Preferably, a retroviral vector is replication defective. This prevents further generation of infectious retroviral particles in the target tissue. Thus, the replication defective virus becomes a “captive” transgene stable incorporated into the target cell genome. This is typically accomplished by deleting the gag, env, and pol genes (along with most of the rest of the viral genome). Heterologous nucleic acids are inserted in place of the deleted viral genes. The heterologous genes may be under the control of the endogenous heterologous promoter, another heterologous promoter active in the target cell, or the retroviral 5′ LTR (the viral LTR is active in diverse tissues).

These delivery vectors described herein can be made target-specific by attaching, for example, a sugar, a glycolipid, or a protein (e.g., an antibody to a target cell receptor).

Reversible delivery expression systems may also be used. The Cre-loxP or FLP/FRT system and other similar systems can be used for reversible delivery-expression of one or more of the above-described nucleic acids. See WO2005/112620, WO2005/039643, US20050130919, US20030022375, US20020022018, US20030027335, and US20040216178. In particular, the reversible delivery-expression system described in US20100284990 can be used to provide a selective or emergency shut-off.

Non-Viral Delivery Methods

Several non-viral methods exist for delivery of anti-BICRA agents including polymeric, biodegradable microparticle, or microcapsule delivery devices known in the art. For example, a colloidal dispersion system may be used for targeted delivery an anti-BICRA agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules.

The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Exemplary phospholipids include egg phosphatidylcholine, dipalmitoylphosphatidylcholine, and distearoyl-phosphatidylcholine. The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255.

### Pharmaceutical Compositions

The pharmaceutical compositions described herein are preferably formulated into pharmaceutical compositions for administration to human subjects in a biologically compatible form suitable for administration in vivo.

The compounds described herein may be used in the form of the free base, in the form of salts, solvates, and as prodrugs. All forms are within the methods described herein. In accordance with the methods of the invention, the described compounds or salts, solvates, or prodrugs thereof may be administered to a patient in a variety of forms depending on the selected route of administration, as will be understood by those skilled in the art. The compounds described herein may be administered, for example, by oral, parenteral, buccal, sublingual, nasal, rectal, patch, pump, intratumoral, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound described herein may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound described herein may be incorporated with an excipient and used in the form of ingestible tablets, buccal tablets, troches, capsules, elixirs, suspensions, syrups, and wafers. A compound described herein may also be administered parenterally. Solutions of a compound described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds described herein, and/or compositions including a compound described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds described herein may be administered initially in a suitable dosage that may be adjusted as required, depending on the clinical response. In general, satisfactory results may be obtained when the compounds described herein are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-50 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### Kits

The invention also features kits including (a) a pharmaceutical composition including an agent that reduces the level and/or activity of BICRA in a cell or subject described herein, and (b) a package insert with instructions to perform any of the methods described herein. In some embodiments, the kit includes (a) a pharmaceutical composition including an agent that reduces the level and/or activity of BICRA in a cell or subject described herein, (b) an additional therapeutic agent (e.g., an anti-cancer agent), and (c) a package insert with instructions to perform any of the methods described herein.

### EXAMPLES

### Example 1—High Density Tiling sgRNA Screen Against Human BAF Complex Subunits in Synovial Sarcoma Cell Line SYO1

The following example shows that BICRA sgRNA inhibits cell growth in synovial sarcoma cells.

Procedure: To perform high density sgRNA tiling screen, an sgRNA library against BAF complex subunits was custom synthesized at Cellecta (Mountain View, Calif.). All BICRA-targeting sgRNAs used in this screen are listed in Table 2. Negative and positive control sgRNA were included in the library. Negative controls consisted of 200 sgRNAs that do not target human genome. The positive controls are sgRNAs targeting essential genes (CDC16, GTF2B, HSPA5, HSPA9, PAFAH1B1, PCNA, POLR2L, RPL9, and SF3A3). All positive and negative control sgRNAs are listed in Table 3. Procedures for virus production, cell infection, and performing the sgRNA screen were previously described (Tsherniak et al, Cell 170:564-576 (2017); Munoz et al, Cancer Discovery 6:900-913 (2016)). For each sgRNA, 50 counts were added to the sequencing counts and for each time point the resulting counts were normalized to the total number of counts. The log 2 of the ratio between the counts (defined as dropout ratio) at day 24 and day 1 post-infection was calculated. For negative control sgRNAs, the 2.5 and 97.5 percentile of the log 2 dropout ratio of all non-targeting sgRNAs was calculated and considered as background (grey box in the graph). Protein domains were obtained from PFAM regions defined for the UNIPROT identifier: Q9NZM4.

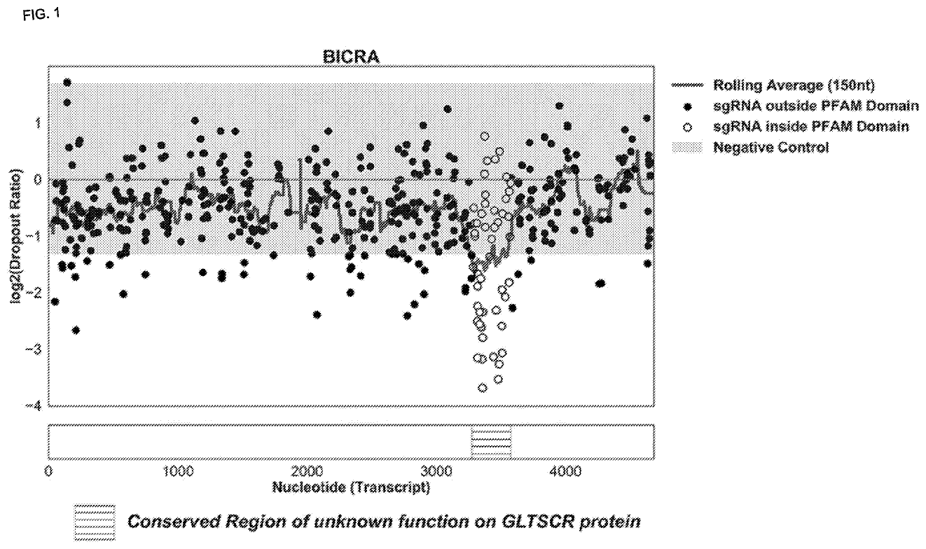

Results: As shown in FIG. 1, targeted inhibition of the GBAF complex component BICRA by sgRNA resulted in growth inhibition of the SYO1 synovial sarcoma cell line, respectively. sgRNAs against other components of the BAF complex resulted in increased proliferation of cells, inhibition of cell growth, or had no effect on SYO1 cells. These data show that targeting various subunits of the GBAF complex represents a therapeutic strategy for the treatment of synovial sarcoma.

### Example 2—High Density Tiling sgRNA Screen Against Human BAF Complex Subunits in Synovial Sarcoma Cell Line HS-SY-II

The following example shows that BICRA sgRNA inhibits cell growth in synovial sarcoma cells.

Procedure: To perform high density sgRNA tiling screen, an sgRNA library against BAF complex subunits was custom synthesized at Cellecta (Mountain View, Calif.). All BICRA-targeting sgRNAs used in this screen are listed in Table 2. Negative and positive control sgRNA were included in the library. Negative controls consisted of 200 sgRNAs that do not target human genome. The positive controls are sgRNAs targeting essential genes (CDC16, GTF2B, HSPA5, HSPA9, PAFAH1B1, PCNA, POLR2L, RPL9, and SF3A3). All positive and negative control sgRNAs are listed in Table 3. Procedures for virus production, cell infection, and performing the sgRNA screen were previously described (Tsherniak et al, Cell 170:564-576 (2017); Munoz et al, Cancer Discovery 6:900-913 (2016)). For each sgRNA, 50 counts were added to the sequencing counts and for each time point the resulting counts were normalized to the total number of counts. The log 2 of the ratio between the counts (defined as dropout ratio) at day 24 and day 1 post-infection was calculated. For negative control sgRNAs, the 2.5 and 97.5 percentile of the log 2 dropout ratio of all non-targeting sgRNAs was calculated and considered as background (grey box in the graph). Protein domains were obtained from PFAM regions defined for the UNIPROT identifier: Q9NZM4.

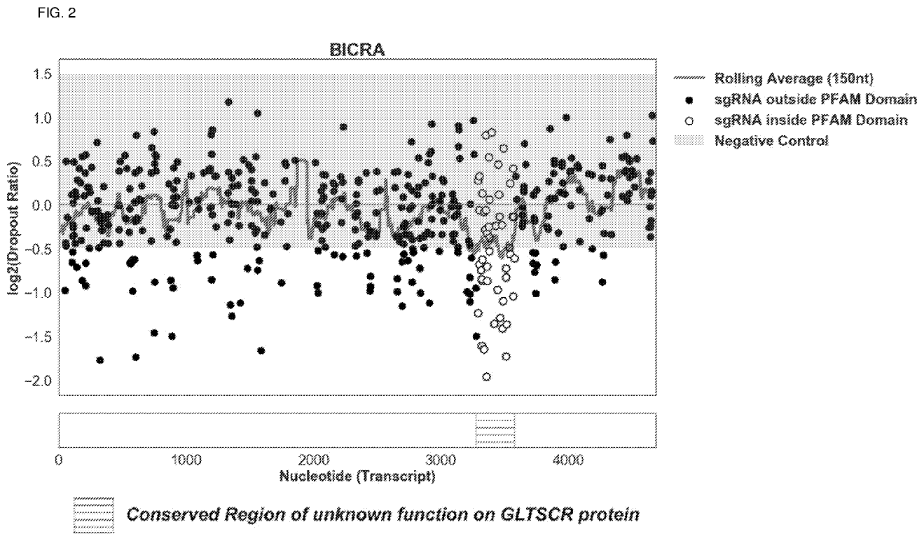

Results: As shown in FIG. 2, targeted inhibition of the GBAF complex component BICRA by sgRNA resulted in growth inhibition of the HS-SY-II synovial sarcoma cell line. sgRNAs against other components of the BAF complex resulted in increased proliferation of cells, inhibition of cell growth, or had no effect on HS-SY-II cells. These data show that targeting various subunits of the GBAF complex represents a therapeutic strategy for the treatment of synovial sarcoma.

TABLE 2
BICRA sgRNA Library

| SEQ ID | | SEQ ID | |
|---|---|---|---|
| NO | Nucleic Acid Sequence | NO | Nucleic Acid Sequence |
| 125 | GGAGGAGGCCACAGCAGAGG | 354 | GGCGTGTTGAGCGCCATGAC |
| 126 | GGTGGAGGATGGAGGGAGGT | 355 | GTTCAGGAGGTGGACGCTCA |
| 127 | CTTGGCCAGGAGCTGGGAGG | 356 | TCCGAGGGTCATCGGCTTCC |
| 128 | GCTGCCTGGGAAGAGGGCTT | 357 | CGGAAGAGGCTGCGATGGGG |
| 129 | ACTGAGCCTGGGCCCCGTGT | 358 | TGGCCACATTCAGGGTCGGG |
| 130 | CTTGGTGGGCAGGTGTGGAG | 359 | CTGCGTCTGTGCTGGTCAGT |
| 131 | CCACCCTCCAGTCACAACGG | 360 | CTGCTTGGCCAGGAGCTGGG |
| 132 | TACCTCACCAGGCCCCTCTG | 361 | GGTGAGCCCCTCAGCGGAGA |
| 133 | TCTGCAGGGAGTCCTGAGTG | 362 | CACGGGCCAGATGAACGGCA |
| 134 | GGTTGGGCTCTGGGTTCAGG | 363 | AACTCTGTGTTCGGAGGCGC |
| 135 | GCCCGGAGAAGATCGTCCTG | 364 | TGCTGCCTTGGTTCAGGAGG |
| 136 | ATCCTCCACCTCCTCTGCTG | 365 | GGAGGGCGCCCTGGTAGACA |
| 137 | AAGGCACTTTGGGCCTCCCC | 366 | GCCATGAACGTCAGGTTCTG |
| 138 | AAGTGGACGAGGCCACCAGC | 367 | TTGTTGACCACGTCCTGGGG |
| 139 | GACCCCCTGGAGGACAGTGG | 368 | TAAATATCGGCTCCTGCTCC |
| 140 | AGGGCCGAGGCCAGCTCCTT | 369 | CCCGACCCTGAATGTGGCCA |
| 141 | AGGGTGCAAGGGTGGCTCTG | 370 | CCCTCGGAAGAGGCTGCGAT |
| 142 | GGAGGCCACAGCAGAGGAGG | 371 | TACCCACCCTCCAGTCACAA |
| 143 | TAGCCCGGAGAAGATCGTCC | 372 | ACCCCCCGCTACCCTCAAGG |
| 144 | AAAGATGCCAGGCAGAGTTG | 373 | GGGGGCCCTTCAGCATACGC |
| 145 | GTGGAGGGTTGGAGGGGGAG | 374 | GCCCAAAGTGCCTTCTATGA |
| 146 | CCTCGGAAGAGGCTGCGATG | 375 | TCAGGGACCAGGTGGAGGGT |
| 147 | CATCCTCCAGAACAAGGCTG | 376 | GTCAGCTGGTAGAGCTTGGG |
| 148 | CCTGGAGGACGTCTCAGAGG | 377 | CTGCCTGGGAAGAGGGCTTG |
| 149 | TGTGCGATGCAGGATCACGT | 378 | TGCTCCTGGAGGAGTCCCGG |
| 150 | TGCCCACCAAGCTTGTGATC | 379 | CAGGCAGTAGGAACTGGTTC |
| 151 | GAAGCTGGCCTGGTCCACGT | 380 | GATGGCGCTCTGCAGGTGCT |
| 152 | GTCCAAGGAGGAGGCGGCAG | 381 | CTTCCTGGAAGATGACATCC |
| 153 | GGACCAGGTGGAGGGTTGGA | 382 | ACAGCTGGCCAAGGAGAAGC |
| 154 | ATCATTACCATCTCCGCTGA | 383 | CTGACCAGCACAGACGCAGC |
| 155 | TGGAGGATGGAGGGAGGTGG | 384 | GAAGTCTAGGTCCACACTGG |
| 156 | TGGAAGGTCCGAGGAGGCGG | 385 | ATGGGTTTGCTCAGGGCCCC |
| 157 | TGGGCAGCTGGAAGGGACCC | 386 | GATGACCTCCTGGATAATCC |
| 158 | GATCATTACCATCTCCGCTG | 387 | GTGCCTTCTATGAAGGTCCT |
| 159 | ACAGCAGAGGAGGTGGAGGA | 388 | CCCCACGGCCATCCTCACTC |
| 160 | TCCAGTTCCCACCCAGCCAG | 389 | AGCTTGTGATCCGGCACGGC |
| 161 | CTGAGTGAGGATGGCCGTGG | 390 | CCTCCTGGATAATCCCGGGG |
| 162 | TGACCTCCTGGATAATCCCG | 391 | CTACCTCACCAGGCCCCTCT |
| 163 | CTCTGCTGGAGGATGTCACA | 392 | CTCTGGGTTCAGGTGGTTGC |
| 164 | GCAAGGGTGGCTCTGAGGGA | 393 | TGCAAGGGTGGCTCTGAGGG |
| 165 | TGCCCCAGGACGATCTTCTC | 394 | CAAACGGCTTGGGCGTCAGC |
| 166 | CTCCTCTGCCGCCTCCTCCT | 395 | TTGCTGGGCTGAAGCGTGGG |
| 167 | GCCTCCTCGGACCTTCCAGA | 396 | TGGTCAACGTCCTGGCGCCG |
| 168 | AGAGGTTCTTCTGGATGACC | 397 | GGCGTCAGCTGGTAGAGCTT |
| 169 | TCTTCTCGAGAGATTTCACC | 398 | GTGCCGGATCACAAGCTTGG |
| 170 | AGCGTCCACCTCCTGAACCA | 399 | TGCAGGGCGTCCTCAAAGGA |
| 171 | TCTGCTTCCAGCCGAGAACA | 400 | GATCGACCGAATGTTCATTC |
| 172 | GGGCCTCCCCGGGATTATCC | 401 | CCTGGGGCAGGAACATCTGC |
| 173 | TGCCGGATCACAAGCTTGGT | 402 | CAGCGTCTGCTCCGTGATGT |
| 174 | AGAGTTCCCATTGAGCGTGG | 403 | TCCATGCAAGAAGTCATTGA |
| 175 | GGTGGCCTCGTCCACTTTGG | 404 | CAGGAAGTCTAGGTCCACAC |
| 176 | TTCCTGGAAGATGACATCCT | 405 | CCCCAGTGACTACCACAAAG |
| 177 | CTGCCCTACCATGTCTACCA | 406 | TGGCCTCTTGGAGGCTCTGC |
| 178 | TCCTGGGCTCTCCTGCGACA | 407 | TTCCTTGCTGGGCTGAAGCG |
| 179 | GCCCCAGGACGATCTTCTCC | 408 | GGGGGTGGTCACCATCTGGA |
| 180 | CTCGAGAAGAGCCTTCGGCT | 409 | CAGAACCAGTTCCTACTGCC |
| 181 | GGTAGTGACGGGGACGAAGA | 410 | CTGTGGCCACCACGCTCAAT |
| 182 | CGGTGAGCGTGTCATCCTGC | 411 | CAGCAAGGTCGTGCACAACA |
| 183 | GAAGAACCTCTCGGCCGCTG | 412 | GACGTTCATGGCGGCGGGGA |
| 184 | CTGCAGGGTGGGCAGCTGGA | 413 | AGTGGACGAGGAGTTTGAGA |
| 185 | TTGGCCAGGAGCTGGGAGGT | 414 | GGGACCAGGTGGAGGGTTGG |
| 186 | AGTGCCTTCTATGAAGGTCC | 415 | CTGTACCAGCGTATGCTGAA |
| 187 | TCCTGCTCCTGGAGGAGTCC | 416 | TCAGTGGGCCCGCCAGGTTC |
| 188 | GCCGTGCCGGATCACAAGCT | 417 | GGGCGCCAGGCAGTAGGAAC |
| 189 | CGGCTGGAAGCAGAGGAGGG | 418 | GGGGGTGTTGAGCATGGCCA |
| 190 | ACCCCCGTCGCCAAAGGAGC | 419 | GACCTTCATAGAAGGCACTT |
| 191 | GTCAGTGGGCAGGCCCCATC | 420 | TCTCGGCTGGAAGCAGAGGA |
| 192 | ACCCTCGGAAGAGGCTGCGA | 421 | GTCTGTGCTGGTCAGTGGGC |
| 193 | GGTGACCCCCTGGAGGACAG | 422 | GCTGGAAGTCGGATGGCGTA |
| 194 | GCAGGGCGTCCTCAAAGGAG | 423 | GTTGAGCATGGCCACGGCGC |
| 195 | CGGAGAGGATGTGCGCGCTG | 424 | AAGCTTGTGATCCGGCACGG |
| 196 | ACTACCTCACCAGGCCCCTC | 425 | GGTAGGGCAGGAGGCGATGC |
| 197 | TCTTGAGCTTGAGCCCGATG | 426 | GTCGGTGCTGCCTGGGAAGA |
| 198 | ATATCGGCTCCTGCTCCTGG | 427 | CATGGGTTTGCTCAGGGCCC |
| 199 | GTTGTGACTGGAGGGTGGGT | 428 | TGGGAACTGGAGCTGGAAGT |
| 200 | AGCGGCCTTGGCCACATTCA | 429 | GAGGCCAGCTCCTTTGGCGA |
| 201 | AGATGCCAGGCAGAGTTGGG | 430 | GTCACCATCTGGAAGGTCCG |
| 202 | GGCCTCCAAGGCCTGCCCAA | 431 | AATCTCTCGAGAAGAGCCTT |
| 203 | GGAAGGGGGTGGTCACCATC | 432 | AGTGAGGATGGCCGTGGGGG |
| 204 | ATGCAGGGCGTCCTCAAAGG | 433 | AAGATGCCAGGCAGAGTTGG |
| 205 | ATCCTCCAGAACAAGGCTGG | 434 | CCTGCAGATGTTCCTGCCCC |
| 206 | TTCTCGGCTGGAAGCAGAGG | 435 | GCAGGAAGCCGGGCTCAGCA |
| 207 | CAGCCCTTCCTGCAGCCTGT | 436 | CGGGCCTCGTAGGTCTTGAT |
| 208 | TGACCGAGGCAGGCACGGAG | 437 | GTCCTGAGTGAGGATGGCCG |
| 209 | CCTCGTAGGTCTTGATGGGC | 438 | ACCTACCGCGAGAACGTGGG |
| 210 | GCTGCAGTGTCACATTGCCC | 439 | CTCCAGTTCCCACCCAGCCA |
| 211 | ACGGGCATCTGGAAGAGCGC | 440 | AACTGGAGCTGGAAGTCGGA |
| 212 | GGGCCTTGTTGACCACGTCC | 441 | GGGAAGGCCGTCTTGTAGTC |
| 213 | AGCTTGGTGGGCAGGTGTGG | 442 | GGGGCCTGGTGAGGTAGTGA |
| 214 | GCTTGACAGTGATGACCTCC | 443 | ACCTGACGTTCATGGCGGCG |
| 215 | GCCTTGTTGACCACGTCCTG | 444 | GCCGTCGGGGGTGTTGAGCA |
| 216 | ACCAGGTGGAGGGTTGGAGG | 445 | GGTAGTCACTGGGGGAGGGG |
| 217 | GTCCTCGTCGGCGTCCAAGG | 446 | CTCCTCCTTGGACGCCGACG |
| 218 | ACCAAGCTTGTGATCCGGCA | 447 | ACTCTGTGTTCGGAGGCGCG |
| 219 | ATGACCTCCTGGATAATCCC | 448 | CGTAGGGGCTGGCAACCTGG |
| 220 | TCATCCTCCAGAACAAGGCT | 449 | CTTGTAGTCGGGGTGCAGGA |
| 221 | GGTGCGTTTCAGCAGCTGCG | 450 | CGTGCCGTTCATCTGGCCCG |
| 222 | TGCTGCTGCCTTGGTTCAGG | 451 | ATGACCAGGCCAGCCCCCTG |
| 223 | GCGATGCAGGGCGTCCTCAA | 452 | GTCCCCGTCACTACCTCACC |
| 224 | GGTCATCCAGAAGAACCTCT | 453 | AGAAGTCATTGAGGGCCTGT |
| 225 | CTTGGCCAGCTGTTTATCCA | 454 | AGCCCAGGATGTCATCTTCC |
| 226 | CGGGGCGCTGACTATGACCG | 455 | CTGAGAGCTGCTGCGGGAGC |
| 227 | CCTCCTCTGAGACGTCCTCC | 456 | AGCTGGAAGTCGGATGGCGT |
| 228 | AAGCCGATGACCCTCGGAAG | 457 | GTGGCCTCGTCCACTTTGGC |
| 229 | TCTAGGTCCACACTGGGGGC | 458 | GGTCGGTGCTGCCTGGGAAG |
| 230 | TCGGTGAGCGTGTCATCCTG | 459 | GGTTCTGGTTTGTGAGGATG |
| 231 | CCCATCGCAGCCTCTTCCGA | 460 | ACTGGAGGGTGGGTAGGCCT |
| 232 | GTGACACTGCAGCCCATCCC | 461 | GAACCCAGAGCCCAACCAGC |
| 233 | CAAGTCCGAGTCGCCCGACG | 462 | CCTGAGTGAGGATGGCCGTG |
| 234 | TCCCCCTCCAACCCTCCACC | 463 | ATTCAGGGTCGGGAGGTTGC |
| 235 | GCTCCGTGATGTTGGCCTCT | 464 | GCCTACCGTGCTGACCCACC |
| 236 | GCTGGTGGCCTCGTCCACTT | 465 | CAGTATGAGAGCAAACTGAG |
| 237 | ACCATCTGGAAGGTCCGAGG | 466 | AGGCCATGCTCAATAAATAT |
| 238 | CCGGGCCTCGTAGGTCTTGA | 467 | GGGCCTGGTGAGGTAGTGAC |
| 239 | GACCTACCGCGAGAACGTGG | 468 | CAGCATCCTGAACCTGCAGC |
| 240 | CCTCCCCGGGATTATCCAGG | 469 | GCCCCAGGACGTGGTCAACA |
| 241 | GGAGAGGATGTGCGCGCTGT | 470 | ATGAGCTGTCCACCTGTGTG |
| 242 | GGGTGCAAGGGTGGCTCTGA | 471 | TCTTGATGGGCGGGCGGTTG |
| 243 | TGAGCTGTCCACCTGTGTGG | 472 | CAGCCTCTTCCGAGGGTCAT |
| 244 | GCAGCTGCTGAAACGCACCC | 473 | CCATGAACGTCAGGTTCTGC |
| 245 | AGCCCGGAGAAGATCGTCCT | 474 | AAGTCGGATGGCGTAGGGGC |
| 246 | TCAGTGGGCAGGCCCCATCT | 475 | CGATGCTGCTGCCTTGGTTC |
| 247 | ACCTTCATAGAAGGCACTTT | 476 | TGGGCGTGGGTGTGCGATGC |
| 248 | CCTCCAAGAGGCCAACATCA | 477 | GGTAGGTCTTGCGCAGTGGC |
| 249 | ACCCAGGTCCAGCTCAGCCT | 478 | GGATCACAAGCTTGGTGGGC |
| 250 | TCCTTGCTGGGCTGAAGCGT | 479 | CTGGTACAGCTCGTCCTCCA |
| 251 | TGTCATCTTCCAGGAAGTCT | 480 | CCCCACGCTTCAGCCCAGCA |
| 252 | TTTGTCATCCAAAACCAGCT | 481 | ACCTTGAGGGTAGCGGGGGG |
| 253 | AGGCCCACAGGCTGCAGGAA | 482 | ACAAAGATGCCAGGCAGAGT |
| 254 | AGCCCGACAGCACCACGTTC | 483 | GACGGCCTTCCCCTCCTTTG |
| 255 | GCTGTGGCCACCACGCTCAA | 484 | CTTGCTGGGCTGAAGCGTGG |
| 256 | CAGGATCTGCCCGCCCACGT | 485 | TCTTCTCCGGGCTAGACGCC |
| 257 | GGTAGACATGGTAGGGCAGG | 486 | AGGCCAGCTCCTTTGGCGAC |
| 258 | CAACGTGGGCGGGCAGATCC | 487 | GGCGTCCTCAAAGGAGGGGA |
| 259 | CCTTGCCTTGGATAAACAGC | 488 | AGGAAGTCTAGGTCCACACT |
| 260 | TCGAGAAGAGCCTTCGGCTG | 489 | GGAGGGCGGGACACACTCTG |
| 261 | CCTTGCTGGGCTGAAGCGTG | 490 | CAGTGTGGACCTAGACTTCC |
| 262 | GGCCTGGTGAGGTAGTGACG | 491 | GTCACATTGCCCAGGCCCAC |
| 263 | TCTCGAGAAGAGCCTTCGGC | 492 | GCTTGGTGGGCAGGTGTGGA |
| 264 | ATTGAGCGTGGTGGCCACAG | 493 | GCCCTCAATGACTTCTTGCA |
| 265 | CTCGTCGGCGTCCAAGGAGG | 494 | CCACCGTTGTGACTGGAGGG |
| 266 | GCTGGCAAAAGCCTTGTTCT | 495 | TGACCAGCACAGACGCAGCG |
| 267 | CAGGAAGGGCTGCACACTCA | 496 | CAGCTGTTTATCCAAGGCAA |
| 268 | GATGAGCTGTCCACCTGTGT | 497 | TGACGGGCACCTGCTTGGCC |
| 269 | CAACATCACGGAGCAGACGC | 498 | CACAGAGTTCCCATTGAGCG |
| 270 | CAGGCCCACAGGCTGCAGGA | 499 | GGTCGTGCACAACACGGCCC |
| 271 | TGCAGGGTGGGCAGCTGGAA | 500 | TGCCATTGGGCAGGCCTTGG |
| 272 | CCTGCCCTACCATGTCTACC | 501 | CACCTGCTTGGCCAGGAGCT |
| 273 | AAAGTGGACGAGGCCACCAG | 502 | CAGGGAGTCCTGAGTGAGGA |
| 274 | CTCAATGGGAACTCTGTGTT | 503 | GGGCGTCAGCTGGTAGAGCT |
| 275 | TCGCGGTAGGTCTTGCGCAG | 504 | CTGTTTATCCAAGGCAAGGG |
| 276 | GGAAGGCCGTCTTGTAGTCG | 505 | GAAGGCACTTTGGGCCTCCC |
| 277 | GCCTTGGCCACATTCAGGGT | 506 | TTCTGGAGGATGATTCCGGA |
| 278 | GCAGAACCTGACGTTCATGG | 507 | AGTAGGAACTGGTTCTGGCC |
| 279 | GTCATCCTGCGGGCTGTCGC | 508 | AGGCCACCGTTGTGACTGGA |
| 280 | CCTCACAAACCAGAACCTGG | 509 | GGATGACGATGCTGCTGCCT |
| 281 | AACACGGGGCCCAGGCTCAG | 510 | CGGGCTGTCGCTGGAGAAGC |
| 282 | CATCCTCACAAACCAGAACC | 511 | GTGCACGACCTTGCTGAGCC |
| 283 | CTCCATGTGCAAGAAGCTTC | 512 | CTGGGGCAGGAACATCTGCA |
| 284 | AGGGGGAGTGGGGGACTTGT | 513 | GTCGCCAAAGGAGCTGGCCT |
| 285 | GCGCTGACTATGACCGAGGC | 514 | TCAAGATCAAGCAGGAAGCC |
| 286 | AAGCGGCCTTGGCCACATTC | 515 | ATCTGGAAGGTCCGAGGAGG |
| 287 | TTCAGGAGGTGGACGCTCAT | 516 | CTCGGCCACCTTGAGGGTAG |
| 288 | TTGTTCTCGGCTGGAAGCAG | 517 | CATGAACGTCAGGTTCTGCG |
| 289 | CTTCTCCAGCGACAGCCCGC | 518 | GGATGATTCCGGACGGCACC |
| 290 | CCTGGTAGACATGGTAGGGC | 519 | CCTCTTGGAGGCTCTGCTGG |
| 291 | GCTGGAGGATGTCACAGGGC | 520 | GAACTCTGTGTTCGGAGGCG |
| 292 | GGCGCCCTGGTAGACATGGT | 521 | ATATTTATTGAGCATGGCCT |
| 293 | GATGCCAGGCAGAGTTGGGG | 522 | CGACTCGGACTTGCGCCGCT |
| 294 | GGTCCACCGTGCCGTTCATC | 523 | CTCACAAACCAGAACCTGGC |
| 295 | CCTTGGCCACATTCAGGGTC | 524 | TTTGTGGTAGTCACTGGGGG |
| 296 | GCCATGCTCAACACCCCCGA | 525 | TCATTACCATCTCCGCTGAG |
| 297 | TGCTGTCGATGGCGCTCTGC | 526 | GAGCATTTGCACAAACACCA |
| 298 | GCCTCGTAGGTCTTGATGGG | 527 | TCGTCCACTTTGGCGGGCAG |
| 299 | GCAGGAAGGGCTGCACACTC | 528 | ATCCTGGGCTCTCCTGCGAC |
| 300 | CTGGAAGTCGGATGGCGTAG | 529 | GCATCTGGAAGAGCGCAGGC |
| 301 | GTAGGGCAGGAGGCGATGCA | 530 | CTCGCCCTGGATGGTGAGCG |
| 302 | TGGCGTAGGGGCTGGCAACC | 531 | TTGAGCATGGCCACGGCGCT |
| 303 | GCTCTGCTGGAGGATGTCAC | 532 | GGGCGTGTTGAGCGCCATGA |
| 304 | TCTGGCCCGGCAGCATGTGC | 533 | ATCCATGCAAGAAGTCATTG |
| 305 | GAGGGGGAGTGGGGGACTTG | 534 | TCGGCCACCTTGAGGGTAGC |
| 306 | AGGTAGTGACGGGGACGAAG | 535 | CCAGCTGTTTATCCAAGGCA |
| 307 | CAACCTCCCGACCCTGAATG | 536 | CTCCCAGCTCCTGGCCAAGC |
| 308 | GCTGGTTGGGCTCTGGGTTC | 537 | ACAAGCTTGGTGGGCAGGTG |
| 309 | GGGCCAGAACGTGGTGCTGT | 538 | GCTGTACCAGCGTATGCTGA |
| 310 | CACCGTTGTGACTGGAGGGT | 539 | CTTGGATAAACAGCTGGCCA |
| 311 | TCTCCTCCTGAATGAACATT | 540 | CGTAGGTCTTGATGGGCGGG |
| 312 | GCAGCCCTTCCTGCAGCCTG | 541 | TCCCCGCCGCCATGAACGTC |
| 313 | TCCTTGGACGCCGACGAGGA | 542 | GAGCCGATATTTATTGAGCA |
| 314 | GACCAGGTGGAGGGTTGGAG | 543 | GAGGCCACCGTTGTGACTGG |
| 315 | CAACCTGGAGGACGTCTCAG | 544 | AAGAAGTCATTGAGGGCCTG |
| 316 | CCGTGATGTTGGCCTCTTGG | 545 | CTCAAGATCAAGCAGGAAGC |
| 317 | TCCTGAGTGAGGATGGCCGT | 546 | CAGGTTCTGGTTTGTGAGGA |
| 318 | GGACACACTCTGTGGCCGGG | 547 | GTCAGGTTCTGCGGGGCCTT |
| 319 | ACTGACCAGCACAGACGCAG | 548 | CAAAGATGCCAGGCAGAGTT |
| 320 | TCCTCCAGAACAAGGCTGGG | 549 | GCAGGCTGCACCGTGAGGAC |
| 321 | GGAGCATTTGCACAAACACC | 550 | TGTTGAGCGCCATGACGGGC |
| 322 | ATCATCCTCCAGAACAAGGC | 551 | GTGCAAATGCTCCAGGAAAC |
| 323 | GGCCTTGTTGACCACGTCCT | 552 | AACCTGACGTTCATGGCGGC |
| 324 | GACACACTCTGTGGCCGGGA | 553 | CTTCCAGATGCCCGTGTCGC |
| 325 | GGGCCAGATGAACGGCACGG | 554 | TGCTGCCTGGGAAGAGGGCT |
| 326 | AGGTTCTGGTTTGTGAGGAT | 555 | ACACTCTGTGGCCGGGAGGG |
| 327 | AATGGGAACTCTGTGTTCGG | 556 | TCGGACTTGCGCCGCTTGGC |
| 328 | GCTCAAGCTCAAGATCAAGC | 557 | CTTGTTCTGGAGGATGATTC |
| 329 | GCACCTGCTTGGCCAGGAGC | 558 | GCAAAAGCCTTGTTCTCGGC |
| 330 | TTGTGGTAGTCACTGGGGGA | 559 | CGATGAGCTGTCCACCTGTG |
| 331 | GAAGCGTGGGGGGCTTCTTC | 560 | GCTGCCCGCCAAAGTGGACG |
| 332 | GTGCCGTTCATCTGGCCCGT | 561 | CCCCCCCAGCCTTGTTCTGG |
| 333 | CAGCGGCCGAGAGGTTCTTC | 562 | GCCGAGGCCACCGTTGTGAC |
| 334 | GACTATGACCGAGGCAGGCA | 563 | CGACCGAATGTTCATTCAGG |
| 335 | AGCTCCTTTGGCGACGGGGG | 564 | CTTCCTGCAGCCTGTGGGCC |
| 336 | GGACGCTCATGGGTTTGCTC | 565 | CCGCCAGGTTCTGGTTTGTG |
| 337 | CCGTCTTGTAGTCGGGGTGC | 566 | TCCAGTCACAACGGTGGCCT |
| 338 | CCCCATCGCAGCCTCTTCCG | 567 | GATATTTATTGAGCATGGCC |
| 339 | TGTGACACTGCAGCCCATCC | 568 | GGCTGCCATTGGGCAGGCCT |
| 340 | GGGGAAGGCCGTCTTGTAGT | 569 | TGGTGGGTCAGCACGGTAGG |
| 341 | CCCGCAGAACCTGACGTTCA | 570 | TTCCTGCAGCCTGTGGGCCT |
| 342 | CATCACGGAGCAGACGCTGG | 571 | GCGCCCTGGTAGACATGGTA |
| 343 | AAGCTGGCCTGGTCCACGTC | 572 | TGCCGGAAGCTTCTTGCACA |
| 344 | TGTGGTAGTCACTGGGGGAG | 573 | CCTCCAGCAGAGCCTCCAAG |
| 345 | GCTGCGTCTGTGCTGGTCAG | 574 | GTAGTCACTGGGGGAGGGGA |
| 346 | GGTGTTTGTGCAAATGCTCC | 575 | CGTCAGGTTCTGCGGGGCCT |
| 347 | GGAAGTCTAGGTCCACACTG | 576 | CAGGTTCAGGATGCTGTCGA |
| 348 | GAACCTGACGTTCATGGCGG | 577 | TGTCGCTGGAGAAGCTGGCC |
| 349 | GTAGTGACGGGGACGAAGAG | 578 | GGCCAGAACGTGGTGCTGTC |
| 350 | GACGCTCATGGGTTTGCTCA | 579 | GAGCTGTCCACCTGTGTGGG |
| 351 | CCTCCAGAACAAGGCTGGGG | 580 | GCTCCAGTTCCCACCCAGCC |
| 352 | CGGAATCATCCTCCAGAACA | 581 | GGCTGGGTGGGAACTGGAGC |
| 353 | GCCTGGTGGGTCAGCACGGT | | |

TABLE 3
Control sgRNA Library

| SEQ | | | |
|---|---|---|---|
| ID | | | |
| NO. | gRNA Label | Gene | Nucleic Acid Sequence |
| 582 | 1\|sg_Non_Targeting_Human_0001\| | Non-Targeting_Human | GTAGCGAACGTGTCCGGCGT |
| | Non_Targeting_Human | | |
| 583 | 1\|sg_Non_Targeting_Human_0002\| | Non-Targeting_Human | GACCGGAACGATCTCGCGTA |
| | Non_Targeting_Human | | |
| 584 | 1\|sg_Non_Targeting_Human_0003\| | Non-Targeting_Human | GGCAGTCGTTCGGTTGATAT |
| | Non_Targeting_Human | | |
| 585 | 1\|sg_Non_Targeting_Human_0004\| | Non-Targeting_Human | GCTTGAGCACATACGCGAAT |
| | Non_Targeting_Human | | |
| 586 | 1\|sg_Non_Targeting_Human_0005\| | Non-Targeting_Human | GTGGTAGAATAACGTATTAC |
| | Non_Targeting_Human | | |
| 587 | 1\|sg_Non_Targeting_Human_0006\| | Non-Targeting_Human | GTCATACATGGATAAGGCTA |
| | Non_Targeting_Human | | |
| 588 | 1\|sg_Non_Targeting_Human_0007\| | Non-Targeting_Human | GATACACGAAGCATCACTAG |
| | Non_Targeting_Human | | |
| 589 | 1\|sg_Non_Targeting_Human_0008\| | Non-Targeting_Human | GAACGTTGGCACTACTTCAC |
| | Non_Targeting_Human | | |
| 590 | 1\|sg_Non_Targeting_Human_0009\| | Non-Targeting_Human | GATCCATGTAATGCGTTCGA |
| | Non_Targeting_Human | | |
| 591 | 1\|sg_Non_Targeting_Human_0010\| | Non-Targeting_Human | GTCGTGAAGTGCATTCGATC |
| | Non_Targeting_Human | | |
| 592 | 1\|sg_Non_Targeting_Human_0011\| | Non-Targeting_Human | GTTCGACTCGCGTGACCGTA |
| | Non_Targeting_Human | | |
| 593 | 1\|sg_Non_Targeting_Human_0012\| | Non-Targeting_Human | GAATCTACCGCAGCGGTTCG |
| | Non_Targeting_Human | | |
| 594 | 1\|sg_Non_Targeting_Human_0013\| | Non-Targeting_Human | GAAGTGACGTCGATTCGATA |
| | Non_Targeting_Human | | |
| 595 | 1\|sg_Non_Targeting_Human_0014\| | Non-Targeting_Human | GCGGTGTATGACAACCGCCG |
| | Non_Targeting_Human | | |
| 596 | 1\|sg_Non_Targeting_Human_0015\| | Non-Targeting_Human | GTACCGCGCCTGAAGTTCGC |
| | Non_Targeting_Human | | |
| 597 | 1\|sg_Non_Targeting_Human_0016\| | Non-Targeting_Human | GCAGCTCGTGTGTCGTACTC |
| | Non_Targeting_Human | | |
| 598 | 1\|sg_Non_Targeting_Human_0017\| | Non-Targeting_Human | GCGCCTTAAGAGTACTCATC |
| | Non_Targeting_Human | | |
| 599 | 1\|sg_Non_Targeting_Human_0018\| | Non-Targeting_Human | GAGTGTCGTCGTTGCTCCTA |
| | Non_Targeting_Human | | |
| 600 | 1\|sg_Non_Targeting_Human_0019\| | Non-Targeting_Human | GCAGCTCGACCTCAAGCCGT |
| | Non_Targeting_Human | | |
| 601 | 1\|sg_Non_Targeting_Human_0020\| | Non-Targeting_Human | GTATCCTGACCTACGCGCTG |
| | Non_Targeting_Human | | |
| 602 | 1\|sg_Non_Targeting_Human_0021\| | Non-Targeting_Human | GTGTATCTCAGCACGCTAAC |
| | Non_Targeting_Human | | |
| 603 | 1\|sg_Non_Targeting_Human_0022\| | Non-Targeting_Human | GTCGTCATACAACGGCAACG |
| | Non_Targeting_Human | | |
| 604 | 1\|sg_Non_Targeting_Human_0023\| | Non-Targeting_Human | GTCGTGCGCTTCCGGCGGTA |
| | Non_Targeting_Human | | |
| 605 | 1\|sg_Non_Targeting_Human_0024\| | Non-Targeting_Human | GCGGTCCTCAGTAAGCGCGT |
| | Non_Targeting_Human | | |
| 606 | 1\|sg_Non_Targeting_Human_0025\| | Non-Targeting_Human | GCTCTGCTGCGGAAGGATTC |
| | Non_Targeting_Human | | |
| 607 | 1\|sg_Non_Targeting_Human_0026\| | Non-Targeting_Human | GCATGGAGGAGCGTCGCAGA |
| | Non_Targeting_Human | | |
| 608 | 1\|sg_Non_Targeting_Human_0027\| | Non-Targeting_Human | GTAGCGCGCGTAGGAGTGGC |
| | Non_Targeting_Human | | |
| 609 | 1\|sg_Non_Targeting_Human_0028\| | Non-Targeting_Human | GATCACCTGCATTCGTACAC |
| | Non_Targeting_Human | | |
| 610 | 1\|sg_Non_Targeting_Human_0029\| | Non-Targeting_Human | GCACACCTAGATATCGAATG |
| | Non_Targeting_Human | | |
| 611 | 1\|sg_Non_Targeting_Human_0030\| | Non-Targeting_Human | GTTGATCAACGCGCTTCGCG |
| | Non_Targeting_Human | | |
| 612 | 1\|sg_Non_Targeting_Human_0031\| | Non-Targeting_Human | GCGTCTCACTCACTCCATCG |
| | Non_Targeting_Human | | |
| 613 | 1\|sg_Non_Targeting_Human_0032\| | Non-Targeting_Human | GCCGACCAACGTCAGCGGTA |
| | Non_Targeting_Human | | |
| 614 | 1\|sg_Non_Targeting_Human_0033\| | Non-Targeting_Human | GGATACGGTGCGTCAATCTA |
| | Non_Targeting_Human | | |
| 615 | 1\|sg_Non_Targeting_Human_0034\| | Non-Targeting_Human | GAATCCAGTGGCGGCGACAA |
| | Non_Targeting_Human | | |
| 616 | 1\|sg_Non_Targeting_Human_0035\| | Non-Targeting_Human | GCACTGTCAGTGCAACGATA |
| | Non_Targeting_Human | | |
| 617 | 1\|sg_Non_Targeting_Human_0036\| | Non-Targeting_Human | GCGATCCTCAAGTATGCTCA |
| | Non_Targeting_Human | | |
| 618 | 1\|sg_Non_Targeting_Human_0037\| | Non-Targeting_Human | GCTAATATCGACACGGCCGC |
| | Non_Targeting_Human | | |
| 619 | 1\|sg_Non_Targeting_Human_0038\| | Non-Targeting_Human | GGAGATGCATCGAAGTCGAT |
| | Non_Targeting_Human | | |
| 620 | 1\|sg_Non_Targeting_Human_0039\| | Non-Targeting_Human | GGATGCACTCCATCTCGTCT |
| | Non_Targeting_Human | | |
| 621 | 1\|sg_Non_Targeting_Human_0040\| | Non-Targeting_Human | GTGCCGAGTAATAACGCGAG |
| | Non_Targeting_Human | | |
| 622 | 1\|sg_Non_Targeting_Human_0041\| | Non-Targeting_Human | GAGATTCCGATGTAACGTAC |
| | Non_Targeting_Human | | |
| 623 | 1\|sg_Non_Targeting_Human_0042\| | Non-Targeting_Human | GTCGTCACGAGCAGGATTGC |
| | Non_Targeting_Human | | |
| 624 | 1\|sg_Non_Targeting_Human_0043\| | Non-Targeting_Human | GCGTTAGTCACTTAGCTCGA |
| | Non_Targeting_Human | | |
| 625 | 1\|sg_Non_Targeting_Human_0044\| | Non-Targeting_Human | GTTCACACGGTGTCGGATAG |
| | Non_Targeting_Human | | |
| 626 | 1\|sg_Non_Targeting_Human_0045\| | Non-Targeting_Human | GGATAGGTGACCTTAGTACG |
| | Non_Targeting_Human | | |
| 627 | 1\|sg_Non_Targeting_Human_0046\| | Non-Targeting_Human | GTATGAGTCAAGCTAATGCG |
| | Non_Targeting_Human | | |
| 628 | 1\|sg_Non_Targeting_Human_0047\| | Non-Targeting_Human | GCAACTATTGGAATACGTGA |
| | Non_Targeting_Human | | |
| 629 | 1\|sg_Non_Targeting_Human_0048\| | Non-Targeting_Human | GTTACCTTCGCTCGTCTATA |
| | Non_Targeting_Human | | |
| 630 | 1\|sg_Non_Targeting_Human_0049\| | Non-Targeting_Human | GTACCGAGCACCACAGGCCG |
| | Non_Targeting_Human | | |
| 631 | 1\|sg_Non_Targeting_Human_0050\| | Non-Targeting_Human | GTCAGCCATCGGATAGAGAT |
| | Non_Targeting_Human | | |
| 632 | 1\|sg_Non_Targeting_Human_0051\| | Non-Targeting_Human | GTACGGCACTCCTAGCCGCT |
| | Non_Targeting_Human | | |
| 633 | 1\|sg_Non_Targeting_Human_0052\| | Non-Targeting_Human | GGTCCTGTCGTATGCTTGCA |
| | Non_Targeting_Human | | |
| 634 | 1\|sg_Non_Targeting_Human_0053\| | Non-Targeting_Human | GCCGCAATATATGCGGTAAG |
| | Non_Targeting_Human | | |
| 635 | 1\|sg_Non_Targeting_Human_0054\| | Non-Targeting_Human | GCGCACGTATAATCCTGCGT |
| | Non_Targeting_Human | | |
| 636 | 1\|sg_Non_Targeting_Human_0055\| | Non-Targeting_Human | GTGCACAACACGATCCACGA |
| | Non_Targeting_Human | | |
| 637 | 1\|sg_Non_Targeting_Human_0056\| | Non-Targeting_Human | GCACAATGTTGACGTAAGTG |
| | Non_Targeting_Human | | |
| 638 | 1\|sg_Non_Targeting_Human_0057\| | Non-Targeting_Human | GTAAGATGCTGCTCACCGTG |
| | Non_Targeting_Human | | |
| 639 | 1\|sg_Non_Targeting_Human_0058\| | Non-Targeting_Human | GTCGGTGATCCAACGTATCG |
| | Non_Targeting_Human | | |
| 640 | 1\|sg_Non_Targeting_Human_0059\| | Non-Targeting_Human | GAGCTAGTAGGACGCAAGAC |
| | Non_Targeting_Human | | |
| 641 | 1\|sg_Non_Targeting_Human_0060\| | Non-Targeting_Human | GTACGTGGAAGCTTGTGGCC |
| | Non_Targeting_Human | | |
| 642 | 1\|sg_Non_Targeting_Human_0061\| | Non-Targeting_Human | GAGAACTGCCAGTTCTCGAT |
| | Non_Targeting_Human | | |
| 643 | 1\|sg_Non_Targeting_Human_0062\| | Non-Targeting_Human | GCCATTCGGCGCGGCACTTC |
| | Non_Targeting_Human | | |
| 644 | 1\|sg_Non_Targeting_Human_0063\| | Non-Targeting_Human | GCACACGACCAATCCGCTTC |
| | Non_Targeting_Human | | |
| 645 | 1\|sg_Non_Targeting_Human_0064\| | Non-Targeting_Human | GAGGTGATCGATTAAGTACA |
| | Non_Targeting_Human | | |
| 646 | 1\|sg_Non_Targeting_Human_0065\| | Non-Targeting_Human | GTCACTCGCAGACGCCTAAC |
| | Non_Targeting_Human | | |
| 647 | 1\|sg_Non_Targeting_Human_0066\| | Non-Targeting_Human | GCGCTACGGAATCATACGTT |
| | Non_Targeting_Human | | |
| 648 | 1\|sg_Non_Targeting_Human_0067\| | Non-Targeting_Human | GGTAGGACCTCACGGCGCGC |
| | Non_Targeting_Human | | |
| 649 | 1\|sg_Non_Targeting_Human_0068\| | Non-Targeting_Human | GAACTGCATCTTGTTGTAGT |
| | Non_Targeting_Human | | |
| 650 | 1\|sg_Non_Targeting_Human_0069\| | Non-Targeting_Human | GATCCTGATCCGGCGGCGCG |
| | Non_Targeting_Human | | |
| 651 | 1\|sg_Non_Targeting_Human_0070\| | Non-Targeting_Human | GGTATGCGCGATCCTGAGTT |
| | Non_Targeting_Human | | |
| 652 | 1\|sg_Non_Targeting_Human_0071\| | Non-Targeting_Human | GCGGAGCTAGAGAGCGGTCA |
| | Non_Targeting_Human | | |
| 653 | 1\|sg_Non_Targeting_Human_0072\| | Non-Targeting_Human | GAATGGCAATTACGGCTGAT |
| | Non_Targeting_Human | | |
| 654 | 1\|sg_Non_Targeting_Human_0073\| | Non-Targeting_Human | GTATGGTGAGTAGTCGCTTG |
| | Non_Targeting_Human | | |
| 655 | 1\|sg_Non_Targeting_Human_0074\| | Non-Targeting_Human | GTGTAATTGCGTCTAGTCGG |
| | Non_Targeting_Human | | |
| 656 | 1\|sg_Non_Targeting_Human_0075\| | Non-Targeting_Human | GGTCCTGGCGAGGAGCCTTG |
| | Non_Targeting_Human | | |
| 657 | 1\|sg_Non_Targeting_Human_0076\| | Non-Targeting_Human | GAAGATAAGTCGCTGTCTCG |
| | Non_Targeting_Human | | |
| 658 | 1\|sg_Non_Targeting_Human_0077\| | Non-Targeting_Human | GTCGGCGTTCTGTTGTGACT |
| | Non_Targeting_Human | | |
| 659 | 1\|sg_Non_Targeting_Human_0078\| | Non-Targeting_Human | GAGGCAAGCCGTTAGGTGTA |
| | Non_Targeting_Human | | |
| 660 | 1\|sg_Non_Targeting_Human_0079\| | Non-Targeting_Human | GCGGATCCAGATCTCATTCG |
| | Non_Targeting_Human | | |
| 661 | 1\|sg_Non_Targeting_Human_0080\| | Non-Targeting_Human | GGAACATAGGAGCACGTAGT |
| | Non_Targeting_Human | | |
| 662 | 1\|sg_Non_Targeting_Human_0081\| | Non-Targeting_Human | GTCATCATTATGGCGTAAGG |
| | Non_Targeting_Human | | |
| 663 | 1\|sg_Non_Targeting_Human_0082\| | Non-Targeting_Human | GCGACTAGCGCCATGAGCGG |
| | Non_Targeting_Human | | |
| 664 | 1\|sg_Non_Targeting_Human_0083\| | Non-Targeting_Human | GGCGAAGTTCGACATGACAC |
| | Non_Targeting_Human | | |
| 665 | 1\|sg_Non_Targeting_Human_0084\| | Non-Targeting_Human | GCTGTCGTGTGGAGGCTATG |
| | Non_Targeting_Human | | |
| 666 | 1\|sg_Non_Targeting_Human_0085\| | Non-Targeting_Human | GCGGAGAGCATTGACCTCAT |
| | Non_Targeting_Human | | |
| 667 | 1\|sg_Non_Targeting_Human_0086\| | Non-Targeting_Human | GACTAATGGACCAAGTCAGT |
| | Non_Targeting_Human | | |
| 668 | 1\|sg_Non_Targeting_Human_0087\| | Non-Targeting_Human | GCGGATTAGAGGTAATGCGG |
| | Non_Targeting_Human | | |
| 669 | 1\|sg_Non_Targeting_Human_0088\| | Non-Targeting_Human | GCCGACGGCAATCAGTACGC |
| | Non_Targeting_Human | | |
| 670 | 1\|sg_Non_Targeting_Human_0089\| | Non-Targeting_Human | GTAACCTCTCGAGCGATAGA |
| | Non_Targeting_Human | | |
| 671 | 1\|sg_Non_Targeting_Human_0090\| | Non-Targeting_Human | GACTTGTATGTGGCTTACGG |
| | Non_Targeting_Human | | |
| 672 | 1\|sg_Non_Targeting_Human_0091\| | Non-Targeting_Human | GTCACTGTGGTCGAACATGT |
| | Non_Targeting_Human | | |
| 673 | 1\|sg_Non_Targeting_Human_0092\| | Non-Targeting_Human | GTACTCCAATCCGCGATGAC |
| | Non_Targeting_Human | | |
| 674 | 1\|sg_Non_Targeting_Human_0093\| | Non-Targeting_Human | GCGTTGGCACGATGTTACGG |
| | Non_Targeting_Human | | |
| 675 | 1\|sg_Non_Targeting_Human_0094\| | Non-Targeting_Human | GAACCAGCCGGCTAGTATGA |
| | Non_Targeting_Human | | |
| 676 | 1\|sg_Non_Targeting_Human_0095\| | Non-Targeting_Human | GTATACTAGCTAACCACACG |
| | Non_Targeting_Human | | |
| 677 | 1\|sg_Non_Targeting_Human_0096\| | Non-Targeting_Human | GAATCGGAATAGTTGATTCG |
| | Non_Targeting_Human | | |
| 678 | 1\|sg_Non_Targeting_Human_0097\| | Non-Targeting_Human | GAGCACTTGCATGAGGCGGT |
| | Non_Targeting_Human | | |
| 679 | 1\|sg_Non_Targeting_Human_0098\| | Non-Targeting_Human | GAACGGCGATGAAGCCAGCC |
| | Non_Targeting_Human | | |
| 680 | 1\|sg_Non_Targeting_Human_0099\| | Non-Targeting_Human | GCAACCGAGATGAGAGGTTC |
| | Non_Targeting_Human | | |
| 681 | 1\|sg_Non_Targeting_Human_0100\| | Non-Targeting_Human | GCAAGATCAATATGCGTGAT |
| | Non_Targeting_Human | | |
| 682 | 1\|sg_Non_Targeting_Human_GA_ | Non-Targeting_Human | ACGGAGGCTAAGCGTCGCAA |
| | 0101\|Non_Targeting_Human | | |
| 683 | 1\|sg_Non_Targeting_Human_GA_ | Non-Targeting_Human | CGCTTCCGCGGCCCGTTCAA |
| | 0102\|Non_Targeting_Human | | |
| 684 | 1\|sg_Non_Targeting_Human_GA_ | Non-Targeting_Human | ATCGTTTCCGCTTAACGGCG |
| | 0103\|Non_Targeting_Human | | |
| 685 | 1\|sg_Non_Targeting_Human_GA_ | Non-Targeting_Human | GTAGGCGCGCCGCTCTCTAC |
| | 0104\|Non_Targeting_Human | | |
| 686 | 1\|sg_Non_Targeting_Human_GA_ | Non-Targeting_Human | CCATATCGGGGCGAGACATG |
| | 0105\|Non_Targeting_Human | | |
| 687 | 1\|sg_Non_Targeting_Human_GA_ | Non-Targeting_Human | TACTAACGCCGCTCCTACAG |
| | 0106\|Non_Targeting_Human | | |
| 688 | 1\|sg_Non_Targeting_Human_GA_ | Non-Targeting_Human | TGAGGATCATGTCGAGCGCC |
| | 0107\|Non_Targeting_Human | | |
| 689 | 1\|sg_Non_Targeting_Human_GA_ | Non-Targeting_Human | GGGCCCGCATAGGATATCGC |
| | 0108\|Non_Targeting_Human | | |
| 690 | 1\|sg_Non_Targeting_Human_GA_ | Non-Targeting_Human | TAGACAACCGCGGAGAATGC |
| | 0109\|Non_Targeting_Human | | |
| 691 | 1\|sg_Non_Targeting_Human_GA_ | Non-Targeting_Human | ACGGGCGGCTATCGCTGACT |
| | 0110\|Non_Targeting_Human | | |
| 692 | 1\|sg_Non_Targeting_Human_GA_ | Non-Targeting_Human | CGCGGAAATTTTACCGACGA |
| | 0111\|Non_Targeting_Human | | |
| 693 | 1\|sg_Non_Targeting_Human_GA_ | Non-Targeting_Human | CTTACAATCGTCGGTCCAAT |
| | 0112\|Non_Targeting_Human | | |
| 694 | 1\|sg_Non_Targeting_Human_GA_ | Non-Targeting_Human | GCGTGCGTCCCGGGTTACCC |
| | 0113\|Non_Targeting_Human | | |
| 695 | 1\|sg_Non_Targeting_Human_GA_ | Non-Targeting_Human | CGGAGTAACAAGCGGACGGA |
| | 0114\|Non_Targeting_Human | | |
| 696 | 1\|sg_Non_Targeting_Human_GA_ | Non-Targeting_Human | CGAGTGTTATACGCACCGTT |
| | 0115\|Non_Targeting_Human | | |
| 697 | 1\|sg_Non_Targeting_Human_GA_ | Non-Targeting_Human | CGACTAACCGGAAACTTTTT |
| | 0116\|Non_Targeting_Human | | |
| 698 | 1\|sg_Non_Targeting_Human_GA_ | Non-Targeting_Human | CAACGGGTTCTCCCGGCTAC |
| | 0117\|Non_Targeting_Human | | |
| 699 | 1\|sg_Non_Targeting_Human_GA_ | Non-Targeting_Human | CAGGAGTCGCCGATACGCGT |
| | 0118\|Non_Targeting_Human | | |
| 700 | 1\|sg_Non_Targeting_Human_GA_ | Non-Targeting_Human | TTCACGTCGTCTCGCGACCA |
| | 0119\|Non_Targeting_Human | | |
| 701 | 1\|sg_Non_Targeting_Human_GA_ | Non-Targeting_Human | GTGTCGGATTCCGCCGCTTA |
| | 0120\|Non_Targeting_Human | | |
| 702 | 1\|sg_Non_Targeting_Human_GA_ | Non-Targeting_Human | CACGAACTCACACCGCGCGA |
| | 0121\|Non_Targeting_Human | | |
| 703 | 1\|sg_Non_Targeting_Human_GA_ | Non-Targeting_Human | CGCTAGTACGCTCCTCTATA |
| | 0122\|Non_Targeting_Human | | |
| 704 | 1\|sg_Non_Targeting_Human_GA_ | Non-Targeting_Human | TCGCGCTTGGGTTATACGCT |
| | 0123\|Non_Targeting_Human | | |
| 705 | 1\|sg_Non_Targeting_Human_GA_ | Non-Targeting_Human | CTATCTCGAGTGGTAATGCG |
| | 0124\|Non_Targeting_Human | | |
| 706 | 1\|sg_Non_Targeting_Human_GA_ | Non-Targeting_Human | AATCGACTCGAACTTCGTGT |
| | 0125\|Non_Targeting_Human | | |
| 707 | 1\|sg_Non_Targeting_Human_GA_ | Non-Targeting_Human | CCCGATGGACTATACCGAAC |
| | 0126\|Non_Targeting_Human | | |
| 708 | 1\|sg_Non_Targeting_Human_GA_ | Non-Targeting_Human | ACGTTCGAGTACGACCAGCT |
| | 0127\|Non_Targeting_Human | | |
| 709 | 1\|sg_Non_Targeting_Human_GA_ | Non-Targeting_Human | CGCGACGACTCAACCTAGTC |
| | 0128\|Non_Targeting_Human | | |
| 710 | 1\|sg_Non_Targeting_Human_GA_ | Non-Targeting_Human | GGTCACCGATCGAGAGCTAG |
| | 0129\|Non_Targeting_Human | | |
| 711 | 1\|sg_Non_Targeting_Human_GA_ | Non-Targeting_Human | CTCAACCGACCGTATGGTCA |
| | 0130\|Non_Targeting_Human | | |
| 712 | 1\|sg_Non_Targeting_Human_GA_ | Non-Targeting_Human | CGTATTCGACTCTCAACGCG |
| | 0131\|Non_Targeting_Human | | |
| 713 | 1\|sg_Non_Targeting_Human_GA_ | Non-Targeting_Human | CTAGCCGCCCAGATCGAGCC |
| | 0132\|Non_Targeting_Human | | |
| 714 | 1\|sg_Non_Targeting_Human_GA_ | Non-Targeting_Human | GAATCGACCGACACTAATGT |
| | 0133\|Non_Targeting_Human | | |
| 715 | 1\|sg_Non_Targeting_Human_GA_ | Non-Targeting_Human | ACTTCAGTTCGGCGTAGTCA |
| | 0134\|Non_Targeting_Human | | |
| 716 | 1\|sg_Non_Targeting_Human_GA_ | Non-Targeting_Human | GTGCGATGTCGCTTCAACGT |
| | 0135\|Non_Targeting_Human | | |
| 717 | 1\|sg_Non_Targeting_Human_GA_ | Non-Targeting_Human | CGCCTAATTTCCGGATCAAT |
| | 0136\|Non_Targeting_Human | | |
| 718 | 1\|sg_Non_Targeting_Human_GA_ | Non-Targeting_Human | CGTGGCCGGAACCGTCATAG |
| | 0137\|Non_Targeting_Human | | |
| 719 | 1\|sg_Non_Targeting_Human_GA_ | Non-Targeting_Human | ACCCTCCGAATCGTAACGGA |
| | 0138\|Non_Targeting_Human | | |
| 720 | 1\|sg_Non_Targeting_Human_GA_ | Non-Targeting_Human | AAACGGTACGACAGCGTGTG |
| | 0139\|Non_Targeting_Human | | |
| 721 | 1\|sg_Non_Targeting_Human_GA_ | Non-Targeting_Human | ACATAGTCGACGGCTCGATT |
| | 0140\|Non_Targeting_Human | | |
| 722 | 1\|sg_Non_Targeting_Human_GA_ | Non-Targeting_Human | GATGGCGCTTCAGTCGTCGG |
| | 0141\|Non_Targeting_Human | | |
| 723 | 1\|sg_Non_Targeting_Human_GA_ | Non-Targeting_Human | ATAATCCGGAAACGCTCGAC |
| | 0142\|Non_Targeting_Human | | |
| 724 | 1\|sg_Non_Targeting_Human_GA_ | Non-Targeting_Human | CGCCGGGCTGACAATTAACG |
| | 0143\|Non_Targeting_Human | | |
| 725 | 1\|sg_Non_Targeting_Human_GA_ | Non-Targeting_Human | CGTCGCCATATGCCGGTGGC |
| | 0144\|Non_Targeting_Human | | |
| 726 | 1\|sg_Non_Targeting_Human_GA_ | Non-Targeting_Human | CGGGCCTATAACACCATCGA |
| | 0145\|Non_Targeting_Human | | |
| 727 | 1\|sg_Non_Targeting_Human_GA_ | Non-Targeting_Human | CGCCGTTCCGAGATACTTGA |
| | 0146\|Non_Targeting_Human | | |
| 728 | 1\|sg_Non_Targeting_Human_GA_ | Non-Targeting_Human | CGGGACGTCGCGAAAATGTA |
| | 0147\|Non_Targeting_Human | | |
| 729 | 1\|sg_Non_Targeting_Human_GA_ | Non-Targeting_Human | TCGGCATACGGGACACACGC |
| | 0148\|Non_Targeting_Human | | |
| 730 | 1\|sg_Non_Targeting_Human_GA_ | Non-Targeting_Human | AGCTCCATCGCCGCGATAAT |
| | 0149\|Non_Targeting_Human | | |
| 731 | 1\|sg_Non_Targeting_Human_GA_ | Non-Targeting_Human | ATCGTATCATCAGCTAGCGC |
| | 0150\|Non_Targeting_Human | | |
| 732 | 1\|sg_Non_Targeting_Human_GA_ | Non-Targeting_Human | TCGATCGAGGTTGCATTCGG |
| | 0151\|Non_Targeting_Human | | |
| 733 | 1\|sg_Non_Targeting_Human_GA_ | Non-Targeting_Human | CTCGACAGTTCGTCCCGAGC |
| | 0152\|Non_Targeting_Human | | |
| 734 | 1\|sg_Non_Targeting_Human_GA_ | Non-Targeting_Human | CGGTAGTATTAATCGCTGAC |
| | 0153\|Non_Targeting_Human | | |
| 735 | 1\|sg_Non_Targeting_Human_GA_ | Non-Targeting_Human | TGAACGCGTGTTTCCTTGCA |
| | 0154\|Non_Targeting_Human | | |
| 736 | 1\|sg_Non_Targeting_Human_GA_ | Non-Targeting_Human | CGACGCTAGGTAACGTAGAG |
| | 0155\|Non_Targeting_Human | | |
| 737 | 1\|sg_Non_Targeting_Human_GA_ | Non-Targeting_Human | CATTGTTGAGCGGGCGCGCT |
| | 0156\|Non_Targeting_Human | | |
| 738 | 1\|sg_Non_Targeting_Human_GA_ | Non-Targeting_Human | CCGCTATTGAAACCGCCCAC |
| | 0157\|Non_Targeting_Human | | |
| 739 | 1\|sg_Non_Targeting_Human_GA_ | Non-Targeting_Human | AGACACGTCACCGGTCAAAA |
| | 0158\|Non_Targeting_Human | | |
| 740 | 1\|sg_Non_Targeting_Human_GA_ | Non-Targeting_Human | TTTACGATCTAGCGGCGTAG |
| | 0159\|Non_Targeting_Human | | |
| 741 | 1\|sg_Non_Targeting_Human_GA_ | Non-Targeting_Human | TTCGCACGATTGCACCTTGG |
| | 0160\|Non_Targeting_Human | | |
| 742 | 1\|sg_Non_Targeting_Human_GA_ | Non-Targeting_Human | GGTTAGAGACTAGGCGCGCG |
| | 0161\|Non_Targeting_Human | | |
| 743 | 1\|sg_Non_Targeting_Human_GA_ | Non-Targeting_Human | CCTCCGTGCTAACGCGGACG |
| | 0162\|Non_Targeting_Human | | |
| 744 | 1\|sg_Non_Targeting_Human_GA_ | Non-Targeting_Human | TTATCGCGTAGTGCTGACGT |
| | 0163\|Non_Targeting_Human | | |
| 745 | 1\|sg_Non_Targeting_Human_GA_ | Non-Targeting_Human | TACGCTTGCGTTTAGCGTCC |
| | 0164\|Non_Targeting_Human | | |
| 746 | 1\|sg_Non_Targeting_Human_GA_ | Non-Targeting_Human | CGCGGCCCACGCGTCATCGC |
| | 0165\|Non_Targeting_Human | | |
| 747 | 1\|sg_Non_Targeting_Human_GA_ | Non-Targeting_Human | AGCTCGCCATGTCGGTTCTC |
| | 0166\|Non_Targeting_Human | | |
| 748 | 1\|sg_Non_Targeting_Human_GA_ | Non-Targeting_Human | AACTAGCCCGAGCAGCTTCG |
| | 0167\|Non_Targeting_Human | | |
| 749 | 1\|sg_Non_Targeting_Human_GA_ | Non-Targeting_Human | CGCAAGGTGTCGGTAACCCT |
| | 0168\|Non_Targeting_Human | | |
| 750 | 1\|sg_Non_Targeting_Human_GA_ | Non-Targeting_Human | CTTCGACGCCATCGTGCTCA |
| | 0169\|Non_Targeting_Human | | |
| 751 | 1\|sg_Non_Targeting_Human_GA_ | Non-Targeting_Human | TCCTGGATACCGCGTGGTTA |
| | 0170\|Non_Targeting_Human | | |
| 752 | 1\|sg_Non_Targeting_Human_GA_ | Non-Targeting_Human | ATAGCCGCCGCTCATTACTT |
| | 0171\|Non_Targeting_Human | | |
| 753 | 1\|sg_Non_Targeting_Human_GA_ | Non-Targeting_Human | GTCGTCCGGGATTACAAAAT |
| | 0172\|Non_Targeting_Human | | |
| 754 | 1\|sg_Non_Targeting_Human_GA_ | Non-Targeting_Human | TAATGCTGCACACGCCGAAT |
| | 0173\|Non_Targeting_Human | | |
| 755 | 1\|sg_Non_Targeting_Human_GA_ | Non-Targeting_Human | TATCGCTTCCGATTAGTCCG |
| | 0174\|Non_Targeting_Human | | |
| 756 | 1\|sg_Non_Targeting_Human_GA_ | Non-Targeting_Human | GTACCATACCGCGTACCCTT |
| | 0175\|Non_Targeting_Human | | |
| 757 | 1\|sg_Non_Targeting_Human_GA_ | Non-Targeting_Human | TAAGATCCGCGGGTGGCAAC |
| | 0176\|Non_Targeting_Human | | |
| 758 | 1\|sg_Non_Targeting_Human_GA_ | Non-Targeting_Human | GTAGACGTCGTGAGCTTCAC |
| | 0177\|Non_Targeting_Human | | |
| 759 | 1\|sg_Non_Targeting_Human_GA_ | Non-Targeting_Human | TCGCGGACATAGGGCTCTAA |
| | 0178\|Non_Targeting_Human | | |
| 760 | 1\|sg_Non_Targeting_Human_GA_ | Non-Targeting_Human | AGCGCAGATAGCGCGTATCA |
| | 0179\|Non_Targeting_Human | | |
| 761 | 1\|sg_Non_Targeting_Human_GA_ | Non-Targeting_Human | GTTCGCTTCGTAACGAGGAA |
| | 0180\|Non_Targeting_Human | | |
| 762 | 1\|sg_Non_Targeting_Human_GA_ | Non-Targeting_Human | GACCCCCGATAACTTTTGAC |
| | 0181\|Non_Targeting_Human | | |
| 763 | 1\|sg_Non_Targeting_Human_GA_ | Non-Targeting_Human | ACGTCCATACTGTCGGCTAC |
| | 0182\|Non_Targeting_Human | | |
| 764 | 1\|sg_Non_Targeting_Human_GA_ | Non-Targeting_Human | GTACCATTGCCGGCTCCCTA |
| | 0183\|Non_Targeting_Human | | |
| 765 | 1\|sg_Non_Targeting_Human_GA_ | Non-Targeting_Human | TGGTTCCGTAGGTCGGTATA |
| | 0184\|Non_Targeting_Human | | |
| 766 | 1\|sg_Non_Targeting_Human_GA_ | Non-Targeting_Human | TCTGGCTTGACACGACCGTT |
| | 0185\|Non_Targeting_Human | | |
| 767 | 1\|sg_Non_Targeting_Human_GA_ | Non-Targeting_Human | CGCTAGGTCCGGTAAGTGCG |
| | 0186\|Non_Targeting_Human | | |
| 768 | 1\|sg_Non_Targeting_Human_GA_ | Non-Targeting_Human | AGCACGTAATGTCCGTGGAT |
| | 0187\|Non_Targeting_Human | | |
| 769 | 1\|sg_Non_Targeting_Human_GA_ | Non-Targeting_Human | AAGGCGCGCGAATGTGGCAG |
| | 0188\|Non_Targeting_Human | | |
| 770 | 1\|sg_Non_Targeting_Human_GA_ | Non-Targeting_Human | ACTGCGGAGCGCCCAATATC |
| | 0189\|Non_Targeting_Human | | |
| 771 | 1\|sg_Non_Targeting_Human_GA_ | Non-Targeting_Human | CGTCGAGTGCTCGAACTCCA |
| | 0190\|Non_Targeting_Human | | |
| 772 | 1\|sg_Non_Targeting_Human_GA_ | Non-Targeting_Human | TCGCAGCGGCGTGGGATCGG |
| | 0191\|Non_Targeting_Human | | |
| 773 | 1\|sg_Non_Targeting_Human_GA_ | Non-Targeting_Human | ATCTGTCCTAATTCGGATCG |
| | 0192\|Non_Targeting_Human | | |
| 774 | 1\|sg_Non_Targeting_Human_GA_ | Non-Targeting_Human | TGCGGCGTAATGCTTGAAAG |
| | 0193\|Non_Targeting_Human | | |
| 775 | 1\|sg_Non_Targeting_Human_GA_ | Non-Targeting_Human | CGAACTTAATCCCGTGGCAA |
| | 0194\|Non_Targeting_Human | | |
| 776 | 1\|sg_Non_Targeting_Human_GA_ | Non-Targeting_Human | GCCGTGTTGCTGGATACGCC |
| | 0195\|Non_Targeting_Human | | |
| 777 | 1\|sg_Non_Targeting_Human_GA_ | Non-Targeting_Human | TACCCTCCGGATACGGACTG |
| | 0196\|Non_Targeting_Human | | |
| 778 | 1\|sg_Non_Targeting_Human_GA_ | Non-Targeting_Human | CCGTTGGACTATGGCGGGTC |
| | 0197\|Non_Targeting_Human | | |
| 779 | 1\|sg_Non_Targeting_Human_GA_ | Non-Targeting_Human | GTACGGGGCGATCATCCACA |
| | 0198\|Non_Targeting_Human | | |
| 780 | 1\|sg_Non_Targeting_Human_GA_ | Non-Targeting_Human | AAGAGTAGTAGACGCCCGGG |
| | 0199\|Non_Targeting_Human | | |
| 781 | 1\|sg_Non_Targeting_Human_GA_ | Non-Targeting_Human | AAGAGCGAATCGATTTCGTG |
| | 0200\|Non_Targeting_Human | | |
| 782 | 3\|sg_hCDC16_CC_1\|CDC16 | CDC16 | TCAACACCAGTGCCTGACGG |
| 783 | 3\|sg_hCDC16_CC_2\|CDC16 | CDC16 | AAAGTAGCTTCACTCTCTCG |
| 784 | 3\|sg_hCDC16_CC_3\|CDC16 | CDC16 | GAGCCAACCAATAGATGTCC |
| 785 | 3\|sg_hCDC16_CC_4\|CDC16 | CDC16 | GCGCCGCCATGAACCTAGAG |
| 786 | 3\|sg_hGTF2B_CC_1\|GTF2B | GTF2B | ACAAAGGTTGGAACAGAACC |
| 787 | 3\|sg_hGTF2B_CC_2\|GTF2B | GTF2B | GGTGACCGGGTTATTGATGT |
| 788 | 3\|sg_hGTF2B_CC_3\|GTF2B | GTF2B | TTAGTGGAGGACTACAGAGC |
| 789 | 3\|sg_hGTF2B_CC_4\|GTF2B | GTF2B | ACATATAGCCCGTAAAGCTG |
| 790 | 3\|sg_hHSPA5_CC_1\|HSPA5 | HSPA5 | CGTTGGCGATGATCTCCACG |
| 791 | 3\|sg_hHSPA5_CC_2\|HSPA5 | HSPA5 | TGGCCTTTTCTACCTCGCGC |
| 792 | 3\|sg_hHSPA5_CC_3\|HSPA5 | HSPA5 | AATGGAGATACTCATCTGGG |
| 793 | 3\|sg_hHSPA5_CC_4\|HSPA5 | HSPA5 | GAAGCCCGTCCAGAAAGTGT |
| 794 | 3\|sg_hHSPA9_CC_1\|HSPA9 | HSPA9 | CAATCTGAGGAACTCCACGA |
| 795 | 3\|sg_hHSPA9_CC_2\|HSPA9 | HSPA9 | AGGCTGCGGCGCCCACGAGA |
| 796 | 3\|sg_hHSPA9_CC_3\|HSPA9 | HSPA9 | ACTTTGACCAGGCCTTGCTA |
| 797 | 3\|sg_hHSPA9_CC_4\|HSPA9 | HSPA9 | ACCTTCCATAACTGCCACGC |
| 798 | 3\|sg_hPAFAH1B1_CC_1\|PAFAH1B1 | PAFAH1B1 | CGAGGCGTACATACCCAAGG |
| 799 | 3\|sg_hPAFAH1B1_CC_2\|PAFAH1B1 | PAFAH1B1 | ATGGTACGGCCAAATCAAGA |
| 800 | 3\|sg_hPAFAH1B1_CC_3\|PAFAH1B1 | PAFAH1B1 | TCTTGTAATCCCATACGCGT |
| 801 | 3\|sg_hPAFAH1B1_CC_4\|PAFAH1B1 | PAFAH1B1 | ATTCACAGGACACAGAGAAT |
| 802 | 3\|sg_hPCNA_CC_1\|PCNA | PCNA | CCAGGGCTCCATCCTCAAGA |
| 803 | 3\|sg_hPCNA_CC_2\|PCNA | PCNA | TGAGCTGCACCAAAGAGACG |
| 804 | 3\|sg_hPCNA_CC_3\|PCNA | PCNA | ATGTCTGCAGATGTACCCCT |
| 805 | 3\|sg_hPCNA_CC_4\|PCNA | PCNA | CGAAGATAACGCGGATACCT |
| 806 | 3\|sg_hPOLR2L_CC_1\|POLR2L | POLR2L | GCTGCAGGCCGAGTACACCG |
| 807 | 3\|sg_hPOLR2L_CC_2\|POLR2L | POLR2L | ACAAGTGGGAGGCTTACCTG |
| 808 | 3\|sg_hPOLR2L_CC_3\|POLR2L | POLR2L | GCAGCGTACAGGGATGATCA |
| 809 | 3\|sg_hPOLR2L_CC_4\|POLR2L | POLR2L | GCAGTAGCGCTTCAGGCCCA |
| 810 | 3\|sg_hRPL9_CC_1\|RPL9 | RPL9 | CAAATGGTGGGGTAACAGAA |
| 811 | 3\|sg_hRPL9_CC_2\|RPL9 | RPL9 | GAAAGGAACTGGCTACCGTT |
| 812 | 3\|sg_hRPL9_CC_3\|RPL9 | RPL9 | AGGGCTTCCGTTACAAGATG |
| 813 | 3\|sg_hRPL9_CC_4\|RL9 | RPL9 | GAACAAGCAACACCTAAAAG |
| 814 | 3\|sg_hSF3A3_CC_1\|SF3A3 | SF3A3 | TGAGGAGAAGGAACGGCTCA |
| 815 | 3\|sg_hSF3A3_CC_2\|SF3A3 | SF3A3 | GGAAGAATGCAGAGTATAAG |
| 816 | 3\|sg_hSF3A3_CC_3\|SF3A3 | SF3A3 | GGAATTTGAGGAACTCCTGA |
| 817 | 3\|sg_hSF3A3_CC_4\|SF3A3 | SF3A3 | GCTCACCGGCCATCCAGGAA |
| 818 | 3\|sg_hSF3B3_CC_1\|SF3B3 | SF3B3 | ACTGGCCAGGAACGATGCGA |
| 819 | 3\|sg_hSF3B3_CC_2\|SF3B3 | SF3B3 | GCAGCTCCAAGATCTTCCCA |
| 820 | 3\|sg_hSF3B3_CC_3\|SF3B3 | SF3B3 | GAATGAGTACACAGAACGGA |
| 821 | 3\|sg_hSF3B3_CC_4\|SF3B3 | SF3B3 | GGAGCAGGACAAGGTCGGGG |

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

What is claimed is:

1. A method of treating soft tissue sarcoma in a subject in need thereof, the method comprising administering to the subject an effective amount of an agent that reduces the level and/or activity of BICRA in the sarcoma.

2. A method of reducing tumor growth of a soft tissue sarcoma in a subject in need thereof, the method comprising administering to the subject an effective amount of an agent that reduces the level and/or activity of BICRA in the tumor.

3. A method of inducing apoptosis in a soft tissue sarcoma cell, the method comprising contacting the cell with an effective amount of an agent that reduces the level and/or activity of BICRA in the cell.

4. A method of reducing the level and/or activity of BICRA in a soft tissue sarcoma cell, the method comprising contacting the cell with an effective amount of an agent that reduces the level and/or activity of BICRA in the cell.

5. The method of claim 3 or 4, wherein the soft tissue sarcoma cell is in a subject.

6. The method of any one of claims 1 to 5, wherein the subject or cell has been identified as expressing SS18-SSX fusion protein or BICRA fusion protein.

7. The method of any one of claims 1 to 6, wherein the effective amount of the agent reduces the level and/or activity of BICRA by at least 5% as compared to a reference.

8. The method of any one of claims 1 to 7, wherein the effective amount of the agent reduces the level and/or activity of BICRA by at least 5% as compared to a reference for at least 12 hours.

9. The method of any one of claims 1 to 8, wherein the level and/or activity of SS18-SSX or BICRA fusion protein is reduced in the subject or cell.

10. The method of any one of claims 1 to 9, wherein the soft tissue sarcoma is adult soft tissue sarcoma.

11. The method of claim 10, wherein the adult soft tissue sarcoma is synovial sarcoma.

12. A method of modulating the activity of an SS18-SSX fusion protein, SS18 wild-type protein, or SSX wild-type protein in a cell, the method comprising contacting the cell with an effective amount of an agent that reduces the level and/or activity of BICRA in the cell.

13. A method of modulating the level and/or activity of an SS18-SSX fusion protein, SS18 wild-type protein, or SSX wild-type protein in a cell or subject, the method comprising contacting the cell with an effective amount of an agent that reduces the level and/or activity of BICRA in a cell or subject.

14. The method of claim 12 or 13, wherein the cell is in a subject.

15. A method of treating a disorder related to an SS18-SSX fusion protein, SS18 wild-type protein, or SSX wild-type protein in a subject in need thereof, the method comprising administering to the subject an effective amount of an agent that reduces the level and/or activity of BICRA in an SS18-SSX fusion protein-expressing cell in the subject.

16. The method of any one of claims 12 to 15, wherein the subject has cancer.

17. The method of claim 16, wherein the cancer expresses SS18-SSX fusion protein and/or the cell or subject has been identified as expressing SS18-SSX fusion protein.

18. The method of any one of claims 15 to 17, wherein the disorder is synovial sarcoma or Ewing's sarcoma.

19. The method of claim 18, wherein the disorder is synovial sarcoma.

20. A method of modulating the activity of a BAF complex in a cell or subject, the method comprising contacting the cell with an effective amount of an agent that reduces the level and/or activity of BICRA in the cell or subject.

21. A method of increasing the level and/or activity of BAF47 in a cell or subject, the method comprising contacting the cell with an effective amount of an agent that reduces the level and/or activity of BICRA in the cell or subject.

22. A method of decreasing Wnt/β-catenin signaling in a cell or subject, the method comprising contacting the cell with an effective amount of an agent that reduces the level and/or activity of BICRA in the cell or subject.

23. A method treating a disorder related to BAF47 in a subject in need thereof, the method comprising administering to the subject an effective amount of an agent that reduces the level and/or activity of BICRA in the subject.

24. The method of claim 23, wherein the disorder related to BAF47 is a cancer or viral infection.

25. The method of claim 24, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer.

26. The method of claim 24, wherein the viral infection is an infection with a virus of the Retroviridae family, Hepadnaviridae family, Flaviviridae family, Adenoviridae family, Herpesviridae family, Papillomaviridae family, Parvoviridae family, Polyomaviridae family, Paramyxoviridae family, or Togaviridae family.

27. A method for treating cancer in a subject in need thereof, the method comprising administering to the subject an effective amount of an agent that reduces the level and/or activity of BICRA in a cancer cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

28. A method of reducing tumor growth of a cancer in a subject in need thereof, the method comprising administering to the subject an effective amount of an agent that reduces the level and/or activity of BICRA in a tumor cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

29. A method of inducing apoptosis in a cancer cell, the method comprising contacting the cell with an effective amount of an agent that reduces the level and/or activity of BICRA in the cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

30. A method of reducing the level and/or activity of BICRA in a cancer cell, the method comprising contacting the cell with an effective amount of an agent that reduces the level and/or activity of BICRA in the cell, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, colorectal cancer, non-small cell lung cancer, stomach cancer, breast cancer, malignant rhabdoid tumor, multiple myeloma, or atypical teratoid rhabdoid tumor.

31. The method of any one of claims 27 to 30, wherein the cancer is a CD8+ T-cell lymphoma, endometrial carcinoma, ovarian carcinoma, bladder cancer, stomach cancer, pancreatic cancer, esophageal cancer, prostate cancer, renal cell carcinoma, melanoma, or colorectal cancer.

32. The method of any one of claims 27 to 31, wherein the cancer is non-small cell lung cancer, stomach cancer, breast cancer, malignant rhabdoid tumor, multiple myeloma, or atypical teratoid rhabdoid tumor.

33. A method of modulating the activity of a BICRA fusion protein in a cell or subject, the method comprising contacting the cell with an effective amount of an agent that reduces the level and/or activity of BICRA in the cell or subject.

34. A method of modulating the level and/or activity of a BICRA fusion protein in a cell or subject, the method comprising contacting the cell with an effective amount of an agent that reduces the level and/or activity of BICRA in the cell or subject.

35. The method of claim 33 or 34, wherein the cell is in a subject.

36. A method of treating a disorder related to a BICRA fusion protein in a subject in need thereof, the method comprising administering to the subject an effective amount of an agent that reduces the level and/or activity of BICRA in a BICRA fusion protein-expressing cell.

37. The method of any one of claims 33 to 36, wherein the subject has cancer.

38. The method of claim 37, wherein the cancer expresses a BICRA fusion protein and/or the cell or subject has been identified as expressing a BICRA fusion protein.

39. The method of any one of claims 36 to 38, wherein the disorder related to a BICRA fusion protein is Ewing's sarcoma, lung cancer, or renal cancer.

40. The method of any one of claims 1 to 39, wherein the method further comprises administering to the subject or contacting the cell with an anticancer therapy.

41. The method of claim 40, wherein the anticancer therapy is a chemotherapeutic or cytotoxic agent or radiotherapy.

42. The method of claim 41, wherein the chemotherapeutic or cytotoxic agent is doxorubicin or ifosfamide.

43. The method of claim 41 or 42, wherein the anticancer therapy and the agent that reduces the level and/or activity of BICRA in a cell are administered within 28 days of each other and each in an amount that together are effective to treat the subject.

44. The method of any one of claims 1 to 43, wherein the subject or cancer has been identified as having an elevated level of an SS18-SSX fusion protein or a BICRA fusion protein as compared to a reference.

45. The method of any one of claims 1 to 44, wherein the subject or cancer has been identified as having a decreased level of SS18 wild-type protein or SSX wild-type protein as compared to a reference.

46. A method of treating a viral infection, the method comprising administering to the subject an effective amount of an agent that reduces the level and/or activity of BICRA in a cell of the subject.

47. The method of claim 46, wherein the viral infection is an infection with a virus of the Retroviridae family, Hepadnaviridae family, Flaviviridae family, Adenoviridae family, Herpesviridae family, Papillomaviridae family, Parvoviridae family, Polyomaviridae family, Paramyxoviridae family, or Togaviridae family.

48. The method of any one of claims 1 to 47, wherein the agent that reduces the level and/or activity of BICRA in a cell is a small molecule compound, an antibody, an enzyme, and/or a polynucleotide.

49. The method of claim 48, wherein the agent that reduces the level and/or activity of BICRA in a cell is an enzyme.

50. The method of claim 49, wherein the enzyme is a clustered regularly interspaced short palindromic repeats (CRISPR)-associated protein, a zinc finger nuclease (ZFN), a transcription activator-like effector nuclease (TALEN), or a meganuclease.

51. The method of claim 50, wherein the CRISPR-associated protein is CRISPR-associated protein 9 (Cas9).

52. The method of claim 48, wherein the agent that reduces the level and/or activity of BICRA in a cell is a polynucleotide.

53. The method of claim 52, wherein the polynucleotide is an antisense nucleic acid, a short interfering RNA (siRNA), a short hairpin RNA (shRNA), a micro RNA (miRNA), a CRISPR/Cas 9 nucleotide, or a ribozyme.

54. The method of claim 52, wherein the polynucleotide comprises a sequence having at least 85% sequence identity to the nucleic acid sequence of any one of SEQ ID NOs: 3-124.

55. The method of claim 54, wherein the polynucleotide comprises a sequence having at least 85% sequence identity to the nucleic acid sequence of any one of SEQ ID NOs: 3-68.

56. The method of claim 48, wherein the agent that reduces the level and/or activity of BICRA in a cell is a small molecule compound.

57. The method of claim 56, wherein the small molecule compound is a small molecule BICRA inhibitor.

58. The method of claim 56 or 57, wherein the small molecule compound is a degrader.

59. The method of claim 58, wherein the degrader has the structure of Formula I:
A-L-B Formula I
wherein
A is a BICRA binding moiety;
L is a linker; and
B is a degradation moiety.

60. The method of claim 59, wherein the degradation moiety is a ubiquitin ligase binding moiety.

61. The method of claim 60, wherein the ubiquitin ligase binding moiety comprises Cereblon ligands, IAP (Inhibitors of Apoptosis) ligands, mouse double minute 2 homolog (MDM2), or von Hippel-Lindau ligands, or derivatives or analogs thereof.

62. The method of claim 60 or 61, wherein the ubiquitin ligase binding moiety has the structure: 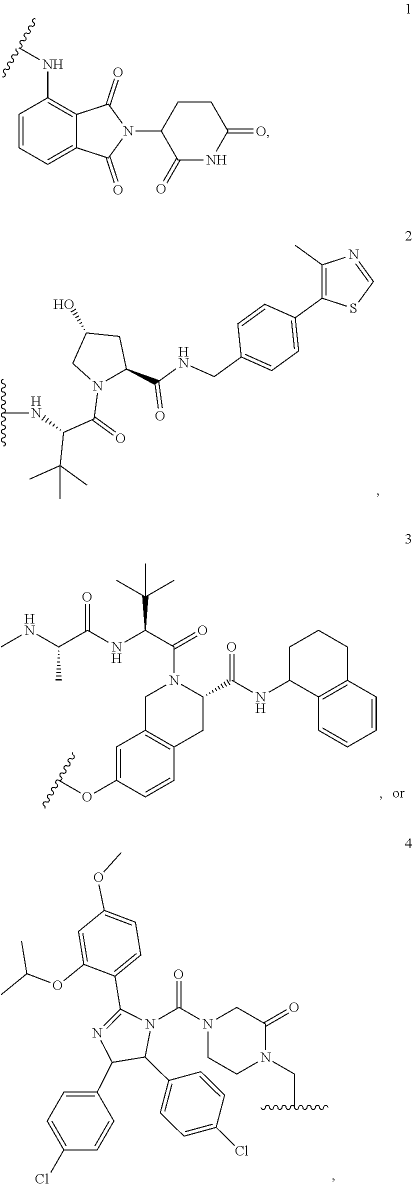 [SMILES: *Nc1cccc2c1C(=O)N(C1CCC(=O)NC1=O)C2=O ; *N[C@H](C(=O)N1C[C@H](O)C[C@H]1C(=O)NCc1ccc(-c2scnc2C)cc1)C(C)(C)C ; *Oc1ccc2c(c1)CN(C(=O)[C@@H](NC(=O)[C@H](C)NC)C(C)(C)C)[C@H](C(=O)NC1CCCc3ccccc31)C2 ; *CN1CCN(C(=O)N2C(c3ccc(OC)cc3OC(C)C)=NC(c3ccc(Cl)cc3)C2c2ccc(Cl)cc2)CC1=O]
or is a derivative or an analog thereof.

63. The method of any one of claims 59 to 62, wherein the linker has the structure of Formula II:
A¹-(B¹)_{f}—(C¹)_{g}—(B²)ₕ-(D)-(B³)ᵢ—(C²)ⱼ—(B⁴)ₖ-A² Formula II
wherein A¹ is a bond between the linker and A; A² is a bond between B and the linker; B¹, B², B³, and B⁴ each, independently, is selected from optionally substituted C₁-C₂ alkyl, optionally substituted C₁-C₃ heteroalkyl, O, S, S(O)₂, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, l, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

64. A method of treating cancer in a subject determined to have an elevated level of SS18-SSX fusion protein, SS18 wild-type protein, SSX wild-type protein, or a BICRA fusion protein, the method comprising administering to the subject an effective amount of an agent that reduces the level and/or activity of BICRA in the cell or subject.

65. The method of claim 64, wherein the level of SS18-SSX fusion protein, SS18 wild-type protein, SSX wild-type protein, or a BICRA fusion protein in the subject is measured in one or more cancer cells.

66. The method of claim 64 or 65, wherein the level of SS18-SSX fusion protein, SS18 wild-type protein, SSX wild-type protein, or a BICRA fusion protein in the subject is measured systemically.

67. A composition comprising an adult soft tissue sarcoma cell and an agent that reduces the level and/or activity of BICRA in a cell.


SEQUENCE LISTING (821 sequences)

SEQ ID NO: 1 - RNA, 5741 nt, Homo sapiens
gcgcggccagagcggccggggacaggctccgaggcaggcccgacccgcctccccggcgccgccgtggctcgacggagaccagctaggctggcccccaagaggaccctttccaagtccccagctgggggccctgtgtagacctggagtggacacgcccctccttcccttcatgattcgtttgtagcgcagtggcgatggatgatgaggatgggagatgcttactagacgtgatttgtgacccacaggccctcaatgacttcttgcatggatccgagaagcttgacagtgatgacctcctggataatcccggggaggcccaaagtgccttctatgaaggtcctgggctccatgtgcaagaagcttccggcaaccacctgaacccagagcccaaccagccggcccccagtgtggacctagacttcctggaagatgacatcctgggctctcctgcgacagggggcggcggcgggggcagtgggggcgctgaccagccctgtgacatcctccagcagagcctccaagaggccaacatcacggagcagacgctggaggccgaggctgagctggacctgggtcccttccagctgcccaccctgcagcctgcggatggcggggcaggcccgacgggcgctggaggggcagcggccgtggctgcggggccccaagccctcttcccaggcagcaccgacctgctggggctgcagggcccgcctaccgtgctgacccaccaggccctggtgccgccccaggacgtggtcaacaaggccctgagtgtgcagcccttcctgcagcctgtgggcctgggcaatgtgacactgcagcccatcccgggcctccaaggcctgcccaatggcagccctgggggtgccacggcggccacactgggcctggcgcccatccaggtggtgggccagcccgtcatggcgctcaacacgcccacctcccagctcctggccaagcaggtgcccgtcagcggctacctggcctcggcggctggcccctcggagcccgtgacgctggcgtcggccggtgtctcgccacagggggctggcctggtcatccagaagaacctctcggccgctgtggccaccacgctcaatgggaactctgtgttcggaggcgcgggggccgcctcggctcccaccgggacgccctcgggacagccgctggcggtggccccaggcctcggctcgtcgccactggtcccggcgcccaacgtgatcctgcatcgcacacccacgcccatccagcccaagcccgcgggggtgctgccgcccaagctctaccagctgacgcccaagccgtttgcgcccgcgggcgccacgctcaccatccagggcgagccgggggcgctcccgcagcagcccaaggccccgcagaacctgacgttcatggcggcggggaaggcgggccagaacgtggtgctgtcgggcttccccgcgcctgcgctgcaagcgaacgtcttcaagcagccaccggccaccaccaccggagcggccccgccgcagccccccggggccctgagcaaacccatgagcgtccacctcctgaaccaaggcagcagcatcgtcatccccgcccagcacatgctgccgggccagaaccagttcctactgcctggcgccccggcggtccagctcccgcagcagctctcagccctgccggccaacgtgggcgggcagatcctggcggccgctgccccccacacaggtggacagctcatcgcgaaccccatcctcacaaaccagaacctggcgggcccactgagcctgggccccgtgttggccccccactccggggcccacagcgcgcacatcctctccgccgctcccatccaggtgggccagcctgcgctcttccagatgcccgtgtcgctggcggcgggcagcctgcccacgcagagccagccagcgcccgccgggccggccgccaccactgtcctccagggggtcaccctgccccccagcgccgtggccatgctcaacacccccgacggcctggtgcagccggccacccctgccgctgccaccggggaggccgcgcctgtcctcacggtgcagcctgccccccaggcgccccccgcggtcagcacacccctgcccctgggcctccagcagccgcaggcgcagcagcccccgcaggcccccaccccacaggccgccgccccgcctcaggccaccaccccccagcccagccctggcctggcgtctagcccggagaagatcgtcctggggcagccgccctctgccacccccacggccatcctcactcaggactccctgcagatgttcctgccccaggagaggagccagcagcccctctccgcagagggcccccacctctccgtgcctgcctcggtcatagtcagcgccccgcctcccgcccaagacccagccccagccacccccgtcgccaaaggagctggcctcggccctcaggcccccgacagccaggcttccccggctccggccccccagatcccggcagcggctccgctgaagggcccaggcccctcttcgtccccgtcactacctcaccaggcccctctgggggacagcccccacctgccctccccacaccccacccggcccccttcccgcccaccctcccggccacagagtgtgtcccgccctccctcagagccacccttgcacccttgccccccaccccaggcccccccaactctgcctggcatctttgtcatccaaaaccagctaggcgttcccccgcctgccagcaacccggcccctactgccccaggcccgccgcagccgcctctccgcccccagtcccagccgcctgagggaccgctgcccccagccccccacctccctccatcctccacctcctctgctgtggcctcctcctctgagacgtcctccaggttgccagcccctacgccatccgacttccagctccagttcccacccagccaggggccccacaagtcccccactccccctccaaccctccacctggtccctgagccggcagcaccccccccaccgcctcctcggaccttccagatggtgaccacccccttcccagcgctgccccagccgaaggctcttctcgagagatttcaccaggtgccgtccggaatcatcctccagaacaaggctgggggggcccctgccgccccgcagacctccaccagcctggggcccctcaccagccccgctgcgtctgtgctggtcagtgggcaggccccatctgggacccccactgcccccagccacgcccccgccccggcacccatggccgccacaggcctccctcctctgcttccagccgagaacaaggcttttgccagcaacctcccgaccctgaatgtggccaaggccgcttcctccgggccagggaagccctccgggctgcagtatgagagcaaactgagtggcctgaagaagccccccacgcttcagcccagcaaggaagcctgtttcctggagcatttgcacaaacaccagggctccgtcctgcaccccgactacaagacggccttcccctcctttgaggacgccctgcatcgcctcctgccctaccatgtctaccagggcgccctcccctcccccagtgactaccacaaagtggacgaggagtttgagacggtctccacgcagctgctgaaacgcacccaggccatgctcaataaatatcggctcctgctcctggaggagtcccggagggtgagcccctcagcggagatggtaatgatcgaccgaatgttcattcaggaggagaagaccacccttgccttggataaacagctggccaaggagaagccggacgagtacgtgtcttcctcccgctcgctcggcctccccatcgcagcctcttccgagggtcatcggcttcccggccacggccccctgtcgtcttcagctcccggggcctccacccagccccctccacacctgcccaccaagcttgtgatccggcacggcggggcaggcggctccccttcggtcacctgggcccgggcgtcctcctccctgtcctcctcttcctcctcctcctctgccgcctcctccttggacgccgacgaggacggccccatgccctcccgcaaccgcccgcccatcaagacctacgaggcccggagccgcatcgggctcaagctcaagatcaagcaggaagccgggctcagcaaggtcgtgcacaacacggccctggaccccgtgcaccagcccccgccaccccccgctaccctcaaggtggccgagcccccgccacggccgccaccaccaccgccgcccacgggccagatgaacggcacggtggaccacccgccgcctgccgcccccgagcgcaagcccctgggcaccgccccgcactgcccgcgcctgccactgcgcaagacctaccgcgagaacgtggggggccctggcgcgccggaggggacgcccgcaggcagggcacggggaggcagcccggcgccgctgcccgccaaagtggacgaggccaccagcgggctcatccgcgagctggcggccgtggaggacgagctgtaccagcgtatgctgaagggccccccgccagagcccgcagccagcgccgcccaaggcaccggggaccccgactgggaggcgcccgggctgccccctgccaagcggcgcaagtccgagtcgcccgacgtggaccaggccagcttctccagcgacagcccgcaggatgacacgctcaccgagcacctgcagagcgccatcgacagcatcctgaacctgcagcaggcccccggccggacgcccgcgccctcgtacccccacgctgcctcggccggcacccccgcatccccgccgcccctgcacaggcccgaggcctacccaccctccagtcacaacggtggcctcggcgccaggacgttgaccagataacaccgggccgcctccccttccccgtcccctcctcccgaagacgccgggacagtcgggtgtccgccctcagcctcctggggactcgagccggggatcccctgacggtttttcttgcctaagttatttgagtcacaaaggcctccttccctgccgcctgcttcagctgggttgctggggggtgggcgtggatttagggagggggctgtgatgtaaaacgtctcccctgccaaaggaggggcaaagtgctgtgtcagttcctgtttcttcccatttcctggcacactctgcccctctgtccgggggacacgcgcatgtgtttgccagggatggggccaccgggttgatgccaacgctccgggtgcctgtcttgtctgtgtggcttctcagatggtggagggtgctgggagctggcagggtccttccagacagtctcagcctctccccgccgcccccaacaggctgtcaaacaaaaccggagagggggtgggggagccagcctcccagcgtgctgtgcccgcaggcacccgtgtgacatccgcacgtccagctccgtgacctgtgtgtgtgtgtgtgtgcacaagtgagtgagagatttcgaacgcccacccctcgactttgaaatctgagcaaaacaagaaactggggtcttcctctcccccgaacctctccccagctagtcttccctctgttcttcctgcctccagccgcccgcgccagattttgaaatctcggagacaaaactagtactgtaagataaatttttttgtactgtatttattgtgtataacgatttttttaaaggagaattctgtacatttagaactcttgtaaattaaaaaccgatcctttttttaaaactgtaaa

SEQ ID NO: 2 - protein, 1560 aa, Homo sapiens [sequence omitted]

SEQ ID NO: 3 - RNA, 20 nt, Artificial Sequence
  Synthetic Construct
ggagggcgccctggtagaca

SEQ ID NO: 4 - RNA, 20 nt, Artificial Sequence
  Synthetic Construct
atatcggctcctgctcctgg

SEQ ID NO: 5 - RNA, 20 nt, Artificial Sequence
  Synthetic Construct
tcctgctcctggaggagtcc

SEQ ID NO: 6 - RNA, 20 nt, Artificial Sequence
  Synthetic Construct
ggcgccctggtagacatggt

SEQ ID NO: 7 - RNA, 20 nt, Artificial Sequence
  Synthetic Construct
tgcagggcgtcctcaaagga

SEQ ID NO: 8 - RNA, 20 nt, Artificial Sequence
  Synthetic Construct
gcagctgctgaaacgcaccc

SEQ ID NO: 9 - RNA, 20 nt, Artificial Sequence
  Synthetic Construct
gatcattaccatctccgctg

SEQ ID NO: 10 - RNA, 20 nt, Artificial Sequence
  Synthetic Construct
cctgccctaccatgtctacc

SEQ ID NO: 11 - RNA, 20 nt, Artificial Sequence
  Synthetic Construct
gaagtctaggtccacactgg

SEQ ID NO: 12 - RNA, 20 nt, Artificial Sequence
  Synthetic Construct
cctggtagacatggtagggc

SEQ ID NO: 13 - RNA, 20 nt, Artificial Sequence
  Synthetic Construct
tcattaccatctccgctgag

SEQ ID NO: 14 - RNA, 20 nt, Artificial Sequence
  Synthetic Construct
ggtagggcaggaggcgatgc

SEQ ID NO: 15 - RNA, 20 nt, Artificial Sequence
  Synthetic Construct
gcagggcgtcctcaaaggag

SEQ ID NO: 16 - RNA, 20 nt, Artificial Sequence
  Synthetic Construct
tcagggaccaggtggagggt

SEQ ID NO: 17 - RNA, 20 nt, Artificial Sequence
  Synthetic Construct
tctgcagggagtcctgagtg

SEQ ID NO: 18 - RNA, 20 nt, Artificial Sequence
  Synthetic Construct
ctgccctaccatgtctacca

SEQ ID NO: 19 - RNA, 20 nt, Artificial Sequence
  Synthetic Construct
gtagggcaggaggcgatgca

SEQ ID NO: 20 - RNA, 20 nt, Artificial Sequence
  Synthetic Construct
aggccatgctcaataaatat

SEQ ID NO: 21 - RNA, 20 nt, Artificial Sequence
  Synthetic Construct
acagctggccaaggagaagc

SEQ ID NO: 22 - RNA, 20 nt, Artificial Sequence
  Synthetic Construct
atgcagggcgtcctcaaagg

SEQ ID NO: 23 - RNA, 20 nt, Artificial Sequence
  Synthetic Construct
gcctcctcggaccttccaga

SEQ ID NO: 24 - RNA, 20 nt, Artificial Sequence
  Synthetic Construct
agaagtcattgagggcctgt

SEQ ID NO: 25 - RNA, 20 nt, Artificial Sequence
  Synthetic Construct
tctcctcctgaatgaacatt

SEQ ID NO: 26 - RNA, 20 nt, Artificial Sequence
  Synthetic Construct
ttctggaggatgattccgga

SEQ ID NO: 27 - RNA, 20 nt, Artificial Sequence
  Synthetic Construct
gcaggaagggctgcacactc

SEQ ID NO: 28 - RNA, 20 nt, Artificial Sequence
  Synthetic Construct
ggggcctggtgaggtagtga

SEQ ID NO: 29 - RNA, 20 nt, Artificial Sequence
  Synthetic Construct
ccttgctgggctgaagcgtg

SEQ ID NO: 30 - RNA, 20 nt, Artificial Sequence
  Synthetic Construct
atcattaccatctccgctga

SEQ ID NO: 31 - RNA, 20 nt, Artificial Sequence
  Synthetic Construct
ttccttgctgggctgaagcg

SEQ ID NO: 32 - RNA, 20 nt, Artificial Sequence
  Synthetic Construct
gacggccttcccctcctttg

SEQ ID NO: 33 - RNA, 20 nt, Artificial Sequence
  Synthetic Construct
gctggtggcctcgtccactt

SEQ ID NO: 34 - RNA, 20 nt, Artificial Sequence
  Synthetic Construct
aagtggacgaggccaccagc

SEQ ID NO: 35 - RNA, 20 nt, Artificial Sequence
  Synthetic Construct
cagctgtttatccaaggcaa

SEQ ID NO: 36 - RNA, 20 nt, Artificial Sequence
  Synthetic Construct
ggtagacatggtagggcagg

SEQ ID NO: 37 - RNA, 20 nt, Artificial Sequence
  Synthetic Construct
ggagcatttgcacaaacacc

SEQ ID NO: 38 - RNA, 20 nt, Artificial Sequence
  Synthetic Construct
ttcaggaggtggacgctcat

SEQ ID NO: 39 - RNA, 20 nt, Artificial Sequence
  Synthetic Construct
tctaggtccacactgggggc

SEQ ID NO: 40 - RNA, 20 nt, Artificial Sequence
  Synthetic Construct
gccccaggacgatcttctcc

SEQ ID NO: 41 - RNA, 20 nt, Artificial Sequence
  Synthetic Construct
gacacactctgtggccggga

SEQ ID NO: 42 - RNA, 20 nt, Artificial Sequence
  Synthetic Construct
cttggccaggagctgggagg

SEQ ID NO: 43 - RNA, 20 nt, Artificial Sequence
  Synthetic Construct
gagctgtccacctgtgtggg

SEQ ID NO: 44 - RNA, 20 nt, Artificial Sequence
  Synthetic Construct
cggaagaggctgcgatgggg

SEQ ID NO: 45 - RNA, 20 nt, Artificial Sequence
  Synthetic Construct
gcgatgcagggcgtcctcaa

SEQ ID NO: 46 - RNA, 20 nt, Artificial Sequence
  Synthetic Construct
gttcaggaggtggacgctca

SEQ ID NO: 47 - RNA, 20 nt, Artificial Sequence
  Synthetic Construct
tccccgccgccatgaacgtc

SEQ ID NO: 48 - RNA, 20 nt, Artificial Sequence
  Synthetic Construct
cttgttctggaggatgattc

SEQ ID NO: 49 - RNA, 20 nt, Artificial Sequence
  Synthetic Construct
ggcctggtgaggtagtgacg

SEQ ID NO: 50 - RNA, 20 nt, Artificial Sequence
  Synthetic Construct
gggcctccccgggattatcc

SEQ ID NO: 51 - RNA, 20 nt, Artificial Sequence
  Synthetic Construct
gtccccgtcactacctcacc

SEQ ID NO: 52 - RNA, 20 nt, Artificial Sequence
  Synthetic Construct
cttgtagtcggggtgcagga

SEQ ID NO: 53 - RNA, 20 nt, Artificial Sequence
  Synthetic Construct
ctctgggttcaggtggttgc

SEQ ID NO: 54 - RNA, 20 nt, Artificial Sequence
  Synthetic Construct
gctgcctgggaagagggctt

SEQ ID NO: 55 - RNA, 20 nt, Artificial Sequence
  Synthetic Construct
cctccccgggattatccagg

SEQ ID NO: 56 - RNA, 20 nt, Artificial Sequence
  Synthetic Construct
tcgagaagagccttcggctg

SEQ ID NO: 57 - RNA, 20 nt, Artificial Sequence
  Synthetic Construct
gttgtgactggagggtgggt

SEQ ID NO: 58 - RNA, 20 nt, Artificial Sequence
  Synthetic Construct
gatgagctgtccacctgtgt

SEQ ID NO: 59 - RNA, 20 nt, Artificial Sequence
  Synthetic Construct
gctggaggatgtcacagggc

SEQ ID NO: 60 - RNA, 20 nt, Artificial Sequence
  Synthetic Construct
tgccggatcacaagcttggt

SEQ ID NO: 61 - RNA, 20 nt, Artificial Sequence
  Synthetic Construct
tccccctccaaccctccacc

SEQ ID NO: 62 - RNA, 20 nt, Artificial Sequence
  Synthetic Construct
agtggacgaggagtttgaga

SEQ ID NO: 63 - RNA, 20 nt, Artificial Sequence
  Synthetic Construct
gtagtgacggggacgaagag

SEQ ID NO: 64 - RNA, 20 nt, Artificial Sequence
  Synthetic Construct
gggtgcaagggtggctctga

SEQ ID NO: 65 - RNA, 20 nt, Artificial Sequence
  Synthetic Construct
gaccccctggaggacagtgg

SEQ ID NO: 66 - RNA, 20 nt, Artificial Sequence
  Synthetic Construct
gagcatttgcacaaacacca

SEQ ID NO: 67 - RNA, 20 nt, Artificial Sequence
  Synthetic Construct
gggcctggtgaggtagtgac

SEQ ID NO: 68 - RNA, 20 nt, Artificial Sequence
  Synthetic Construct
gggccttgttgaccacgtcc

SEQ ID NO: 69 - RNA, 20 nt, Artificial Sequence
  Synthetic Construct
gcccaaagtgccttctatga

SEQ ID NO: 70 - RNA, 20 nt, Artificial Sequence
  Synthetic Construct
caacatcacggagcagacgc

SEQ ID NO: 71 - RNA, 20 nt, Artificial Sequence
  Synthetic Construct
tgccggaagcttcttgcaca

SEQ ID NO: 72 - RNA, 20 nt, Artificial Sequence
  Synthetic Construct
ccgtgatgttggcctcttgg

SEQ ID NO: 73 - RNA, 20 nt, Artificial Sequence
  Synthetic Construct
gctccgtgatgttggcctct

SEQ ID NO: 74 - RNA, 20 nt, Artificial Sequence
  Synthetic Construct
cagcgtctgctccgtgatgt

SEQ ID NO: 75 - RNA, 20 nt, Artificial Sequence
  Synthetic Construct
tgacctcctggataatcccg

SEQ ID NO: 76 - RNA, 20 nt, Artificial Sequence
  Synthetic Construct
ggccttgttgaccacgtcct

SEQ ID NO: 77 - RNA, 20 nt, Artificial Sequence
  Synthetic Construct
ctgtggccaccacgctcaat

SEQ ID NO: 78 - RNA, 20 nt, Artificial Sequence
  Synthetic Construct
tgacgggctggcccaccacc

SEQ ID NO: 79 - RNA, 22 nt, Artificial Sequence
  Synthetic Construct
ccacacagcggagggaggcggc

SEQ ID NO: 80 - RNA, 22 nt, Artificial Sequence
  Synthetic Construct
ggggagagcgagagcccggcug

SEQ ID NO: 81 - RNA, 22 nt, Artificial Sequence
  Synthetic Construct
ccuccuuuccgaggggcgucgu

SEQ ID NO: 82 - RNA, 22 nt, Artificial Sequence
  Synthetic Construct
uucuucagcggacucaguuugc

SEQ ID NO: 83 - RNA, 22 nt, Artificial Sequence
  Synthetic Construct
ugcaccgucucgacaggcgcgg

SEQ ID NO: 84 - RNA, 22 nt, Artificial Sequence
  Synthetic Construct
uuauccaaccgaaggguggucu

SEQ ID NO: 85 - RNA, 22 nt, Artificial Sequence
  Synthetic Construct
ucucucacagucuugugcacac

SEQ ID NO: 86 - RNA, 22 nt, Artificial Sequence
  Synthetic Construct
ugucgaugcgccucugcaggug

SEQ ID NO: 87 - RNA, 22 nt, Artificial Sequence
  Synthetic Construct
ugcacacagugacacacacagg

SEQ ID NO: 88 - RNA, 22 nt, Artificial Sequence
  Synthetic Construct
ugucaagcaagucggauccaug

SEQ ID NO: 89 - RNA, 22 nt, Artificial Sequence
  Synthetic Construct
ugcuccagcuuacaggcuuccu

SEQ ID NO: 90 - RNA, 22 nt, Artificial Sequence
  Synthetic Construct
uugugcaccggcucgcugagcc

SEQ ID NO: 91 - RNA, 22 nt, Artificial Sequence
  Synthetic Construct
ugugcacagugacacacacaca

SEQ ID NO: 92 - RNA, 19 nt, Artificial Sequence
  Synthetic Construct
ggagaattctgtacattta

SEQ ID NO: 93 - RNA, 19 nt, Artificial Sequence
  Synthetic Construct
gtataacgatttttttaaa

SEQ ID NO: 94 - RNA, 19 nt, Artificial Sequence
  Synthetic Construct
ctttgaaatctgagcaaaa

SEQ ID NO: 95 - RNA, 19 nt, Artificial Sequence
  Synthetic Construct
ctgtaagataaattttttt

SEQ ID NO: 96 - RNA, 19 nt, Artificial Sequence
  Synthetic Construct
catttagaactcttgtaaa

SEQ ID NO: 97 - RNA, 19 nt, Artificial Sequence
  Synthetic Construct
gtgatgacctcctggataa

SEQ ID NO: 98 - RNA, 19 nt, Artificial Sequence
  Synthetic Construct
gcatctttgtcatccaaaa

SEQ ID NO: 99 - RNA, 19 nt, Artificial Sequence
  Synthetic Construct
cccaggccatgctcaataa

SEQ ID NO: 100 - RNA, 19 nt, Artificial Sequence
  Synthetic Construct
ggccatgctcaataaatat

SEQ ID NO: 101 - RNA, 19 nt, Artificial Sequence
  Synthetic Construct
cctcagcggagatggtaat

SEQ ID NO: 102 - RNA, 19 nt, Artificial Sequence
  Synthetic Construct
ccacccttgccttggataa

SEQ ID NO: 103 - RNA, 19 nt, Artificial Sequence
  Synthetic Construct
cacccctcgactttgaaat

SEQ ID NO: 104 - RNA, 19 nt, Artificial Sequence
  Synthetic Construct
ccgcgccagattttgaaat

SEQ ID NO: 105 - RNA, 19 nt, Artificial Sequence
  Synthetic Construct
cctgtttcctggagcattt

SEQ ID NO: 106 - RNA, 19 nt, Artificial Sequence
  Synthetic Construct
ggcccaaagtgccttctat

SEQ ID NO: 107 - RNA, 19 nt, Artificial Sequence
  Synthetic Construct
ccaggacgttgaccagata

SEQ ID NO: 108 - RNA, 19 nt, Artificial Sequence
  Synthetic Construct
ctgtatttattgtgtataa

SEQ ID NO: 109 - RNA, 19 nt, Artificial Sequence
  Synthetic Construct
ccggaatcatcctccagaa

SEQ ID NO: 110 - RNA, 19 nt, Artificial Sequence
  Synthetic Construct
ccaggccatgctcaataaa

SEQ ID NO: 111 - RNA, 19 nt, Artificial Sequence
  Synthetic Construct
caggccatgctcaataaat

SEQ ID NO: 112 - RNA, 49 nt, Artificial Sequence
  Synthetic Construct
gaggatgggagatgcttactatcaagagtagtaagcatctcccatcctc

SEQ ID NO: 113 - RNA, 49 nt, Artificial Sequence
  Synthetic Construct
ggatgggagatgcttactagatcaagagtctagtaagcatctcccatcc

SEQ ID NO: 114 - RNA, 49 nt, Artificial Sequence
  Synthetic Construct
gatgcttactagacgtgattttcaagagaaatcacgtctagtaagcatc

SEQ ID NO: 115 - RNA, 49 nt, Artificial Sequence
  Synthetic Construct
ggatccgagaagcttgacagttcaagagactgtcaagcttctcggatcc

SEQ ID NO: 116 - RNA, 49 nt, Artificial Sequence
  Synthetic Construct
gaagcttgacagtgatgaccttcaagagaggtcatcactgtcaagcttc

SEQ ID NO: 117 - RNA, 49 nt, Artificial Sequence
  Synthetic Construct
ggcccaaagtgccttctatgatcaagagtcatagaaggcactttgggcc

SEQ ID NO: 118 - RNA, 49 nt, Artificial Sequence
  Synthetic Construct
gcccaaagtgccttctatgaatcaagagttcatagaaggcactttgggc

SEQ ID NO: 119 - RNA, 49 nt, Artificial Sequence
  Synthetic Construct
gcaaactgagtggcctgaagatcaagagtcttcaggccactcagtttgc

SEQ ID NO: 120 - RNA, 49 nt, Artificial Sequence
  Synthetic Construct
ggtaatgatcgaccgaatgtttcaagagaacattcggtcgatcattacc

SEQ ID NO: 121 - RNA, 49 nt, Artificial Sequence
  Synthetic Construct
gccaggacgttgaccagataatcaagagttatctggtcaacgtcctggc

SEQ ID NO: 122 - RNA, 49 nt, Artificial Sequence
  Synthetic Construct
gtgcacaagtgagtgagagattcaagagatctctcactcacttgtgcac

SEQ ID NO: 123 - RNA, 49 nt, Artificial Sequence
  Synthetic Construct
gcacaagtgagtgagagattttcaagagaaatctctcactcacttgtgc

SEQ ID NO: 124 - RNA, 49 nt, Artificial Sequence
  Synthetic Construct
gctagtcttccctctgttctttcaagagaagaacagagggaagactagc

SEQ ID NO: 125 - RNA, 20 nt, Artificial Sequence
  Synthetic Construct
ggaggaggccacagcagagg

SEQ ID NO: 126 - RNA, 20 nt, Artificial Sequence
  Synthetic Construct
ggtggaggatggagggaggt

SEQ ID NO: 127 - RNA, 20 nt, Artificial Sequence
  Synthetic Construct
cttggccaggagctgggagg

SEQ ID NO: 128 - RNA, 20 nt, Artificial Sequence
  Synthetic Construct
gctgcctgggaagagggctt

SEQ ID NO: 129 - RNA, 20 nt, Artificial Sequence
  Synthetic Construct
actgagcctgggccccgtgt

SEQ ID NO: 130 - RNA, 20 nt, Artificial Sequence
  Synthetic Construct
cttggtgggcaggtgtggag

SEQ ID NO: 131 - RNA, 20 nt, Artificial Sequence
  Synthetic Construct
ccaccctccagtcacaacgg

SEQ ID NO: 132 - RNA, 20 nt, Artificial Sequence
  Synthetic Construct
tacctcaccaggcccctctg

SEQ ID NO: 133 - RNA, 20 nt, Artificial Sequence
  Synthetic Construct
tctgcagggagtcctgagtg

SEQ ID NO: 134 - RNA, 20 nt, Artificial Sequence
  Synthetic Construct
ggttgggctctgggttcagg

SEQ ID NO: 135 - RNA, 20 nt, Artificial Sequence
  Synthetic Construct
gcccggagaagatcgtcctg

SEQ ID NO: 136 - RNA, 20 nt, Artificial Sequence
  Synthetic Construct
atcctccacctcctctgctg

SEQ ID NO: 137 - RNA, 20 nt, Artificial Sequence
  Synthetic Construct
aaggcactttgggcctcccc

SEQ ID NO: 138 - RNA, 20 nt, Artificial Sequence
  Synthetic Construct
aagtggacgaggccaccagc

SEQ ID NO: 139 - RNA, 20 nt, Artificial Sequence
  Synthetic Construct
gaccccctggaggacagtgg

SEQ ID NO: 140 - RNA, 20 nt, Artificial Sequence
  Synthetic Construct
agggccgaggccagctcctt

SEQ ID NO: 141 - RNA, 20 nt, Artificial Sequence
  Synthetic Construct
agggtgcaagggtggctctg

SEQ ID NO: 142 - RNA, 20 nt, Artificial Sequence
  Synthetic Construct
ggaggccacagcagaggagg

SEQ ID NO: 143 - RNA, 20 nt, Artificial Sequence
  Synthetic Construct
tagcccggagaagatcgtcc

SEQ ID NO: 144 - RNA, 20 nt, Artificial Sequence
  Synthetic Construct
aaagatgccaggcagagttg

SEQ ID NO: 145 - RNA, 20 nt, Artificial Sequence
  Synthetic Construct
gtggagggttggagggggag

SEQ ID NO: 146 - RNA, 20 nt, Artificial Sequence
  Synthetic Construct
cctcggaagaggctgcgatg

SEQ ID NO: 147 - RNA, 20 nt, Artificial Sequence
  Synthetic Construct
catcctccagaacaaggctg

SEQ ID NO: 148 - RNA, 20 nt, Artificial Sequence
  Synthetic Construct
cctggaggacgtctcagagg

SEQ ID NO: 149 - RNA, 20 nt, Artificial Sequence
  Synthetic Construct
tgtgcgatgcaggatcacgt

SEQ ID NO: 150 - RNA, 20 nt, Artificial Sequence
  Synthetic Construct
tgcccaccaagcttgtgatc

SEQ ID NO: 151 - RNA, 20 nt, Artificial Sequence
  Synthetic Construct
gaagctggcctggtccacgt

SEQ ID NO: 152 - RNA, 20 nt, Artificial Sequence
  Synthetic Construct
gtccaaggaggaggcggcag

SEQ ID NO: 153 - RNA, 20 nt, Artificial Sequence
  Synthetic Construct
ggaccaggtggagggttgga

SEQ ID NO: 154 - RNA, 20 nt, Artificial Sequence
  Synthetic Construct
atcattaccatctccgctga

SEQ ID NO: 155 - RNA, 20 nt, Artificial Sequence
  Synthetic Construct
tggaggatggagggaggtgg

SEQ ID NO: 156 - RNA, 20 nt, Artificial Sequence
  Synthetic Construct
tggaaggtccgaggaggcgg

SEQ ID NO: 157 - RNA, 20 nt, Artificial Sequence
  Synthetic Construct
tgggcagctggaagggaccc

SEQ ID NO: 158 - RNA, 20 nt, Artificial Sequence
  Synthetic Construct
gatcattaccatctccgctg

SEQ ID NO: 159 - RNA, 20 nt, Artificial Sequence
  Synthetic Construct
acagcagaggaggtggagga

SEQ ID NO: 160 - RNA, 20 nt, Artificial Sequence
  Synthetic Construct
tccagttcccacccagccag

SEQ ID NO: 161 - RNA, 20 nt, Artificial Sequence
  Synthetic Construct
ctgagtgaggatggccgtgg

SEQ ID NO: 162 - RNA, 20 nt, Artificial Sequence
  Synthetic Construct
tgacctcctggataatcccg

SEQ ID NO: 163 - RNA, 20 nt, Artificial Sequence
  Synthetic Construct
ctctgctggaggatgtcaca

SEQ ID NO: 164 - RNA, 20 nt, Artificial Sequence
  Synthetic Construct
gcaagggtggctctgaggga

SEQ ID NO: 165 - RNA, 20 nt, Artificial Sequence
  Synthetic Construct
tgccccaggacgatcttctc

SEQ ID NO: 166 - RNA, 20 nt, Artificial Sequence
  Synthetic Construct
ctcctctgccgcctcctcct

SEQ ID NO: 167 - RNA, 20 nt, Artificial Sequence
  Synthetic Construct
gcctcctcggaccttccaga

SEQ ID NO: 168 - RNA, 20 nt, Artificial Sequence
  Synthetic Construct
agaggttcttctggatgacc

SEQ ID NO: 169 - RNA, 20 nt, Artificial Sequence
  Synthetic Construct
tcttctcgagagatttcacc

SEQ ID NO: 170 - RNA, 20 nt, Artificial Sequence
  Synthetic Construct
agcgtccacctcctgaacca

SEQ ID NO: 171 - RNA, 20 nt, Artificial Sequence
  Synthetic Construct
tctgcttccagccgagaaca

SEQ ID NO: 172 - RNA, 20 nt, Artificial Sequence
  Synthetic Construct
gggcctccccgggattatcc

SEQ ID NO: 173 - RNA, 20 nt, Artificial Sequence
  Synthetic Construct
tgccggatcacaagcttggt

SEQ ID NO: 174 - RNA, 20 nt, Artificial Sequence
  Synthetic Construct
agagttcccattgagcgtgg

SEQ ID NO: 175 - RNA, 20 nt, Artificial Sequence
  Synthetic Construct
ggtggcctcgtccactttgg

SEQ ID NO: 176 - RNA, 20 nt, Artificial Sequence
  Synthetic Construct
ttcctggaagatgacatcct

SEQ ID NO: 177 - RNA, 20 nt, Artificial Sequence
  Synthetic Construct
ctgccctaccatgtctacca

SEQ ID NO: 178 - RNA, 20 nt, Artificial Sequence
  Synthetic Construct
tcctgggctctcctgcgaca

SEQ ID NO: 179 - RNA, 20 nt, Artificial Sequence
  Synthetic Construct
gccccaggacgatcttctcc

SEQ ID NO: 180 - RNA, 20 nt, Artificial Sequence
  Synthetic Construct
ctcgagaagagccttcggct

SEQ ID NO: 181 - RNA, 20 nt, Artificial Sequence
  Synthetic Construct
ggtagtgacggggacgaaga

SEQ ID NO: 182 - RNA, 20 nt, Artificial Sequence
  Synthetic Construct
cggtgagcgtgtcatcctgc

SEQ ID NO: 183 - RNA, 20 nt, Artificial Sequence
  Synthetic Construct
gaagaacctctcggccgctg

SEQ ID NO: 184 - RNA, 20 nt, Artificial Sequence
  Synthetic Construct
ctgcagggtgggcagctgga

SEQ ID NO: 185 - RNA, 20 nt, Artificial Sequence
  Synthetic Construct
ttggccaggagctgggaggt

SEQ ID NO: 186 - RNA, 20 nt, Artificial Sequence
  Synthetic Construct
agtgccttctatgaaggtcc

SEQ ID NO: 187 - RNA, 20 nt, Artificial Sequence
  Synthetic Construct
tcctgctcctggaggagtcc

SEQ ID NO: 188 - RNA, 20 nt, Artificial Sequence
  Synthetic Construct
gccgtgccggatcacaagct

SEQ ID NO: 189 - RNA, 20 nt, Artificial Sequence
  Synthetic Construct
cggctggaagcagaggaggg

SEQ ID NO: 190 - RNA, 20 nt, Artificial Sequence
  Synthetic Construct
acccccgtcgccaaaggagc

SEQ ID NO: 191 - RNA, 20 nt, Artificial Sequence
  Synthetic Construct
gtcagtgggcaggccccatc

SEQ ID NO: 192 - RNA, 20 nt, Artificial Sequence
  Synthetic Construct
accctcggaagaggctgcga

SEQ ID NO: 193 - RNA, 20 nt, Artificial Sequence
  Synthetic Construct
ggtgaccccctggaggacag

SEQ ID NO: 194 - RNA, 20 nt, Artificial Sequence
  Synthetic Construct
gcagggcgtcctcaaaggag

SEQ ID NO: 195 - RNA, 20 nt, Artificial Sequence
  Synthetic Construct
cggagaggatgtgcgcgctg

SEQ ID NO: 196 - RNA, 20 nt, Artificial Sequence
  Synthetic Construct
actacctcaccaggcccctc

SEQ ID NO: 197 - RNA, 20 nt, Artificial Sequence
  Synthetic Construct
tcttgagcttgagcccgatg

SEQ ID NO: 198 - RNA, 20 nt, Artificial Sequence
  Synthetic Construct
atatcggctcctgctcctgg

SEQ ID NO: 199 - RNA, 20 nt, Artificial Sequence
  Synthetic Construct
gttgtgactggagggtgggt

SEQ ID NO: 200 - RNA, 20 nt, Artificial Sequence
  Synthetic Construct
agcggccttggccacattca

SEQ ID NO: 201 - RNA, 20 nt, Artificial Sequence
  Synthetic Construct
agatgccaggcagagttggg

SEQ ID NO: 202 - RNA, 20 nt, Artificial Sequence
  Synthetic Construct
ggcctccaaggcctgcccaa

SEQ ID NO: 203 - RNA, 20 nt, Artificial Sequence
  Synthetic Construct
ggaagggggtggtcaccatc

SEQ ID NO: 204 - RNA, 20 nt, Artificial Sequence
  Synthetic Construct
atgcagggcgtcctcaaagg

SEQ ID NO: 205 - RNA, 20 nt, Artificial Sequence
  Synthetic Construct
atcctccagaacaaggctgg

SEQ ID NO: 206 - RNA, 20 nt, Artificial Sequence
  Synthetic Construct
ttctcggctggaagcagagg

SEQ ID NO: 207 - RNA, 20 nt, Artificial Sequence
  Synthetic Construct
cagcccttcctgcagcctgt

SEQ ID NO: 208 - RNA, 20 nt, Artificial Sequence
  Synthetic Construct
tgaccgaggcaggcacggag

SEQ ID NO: 209 - RNA, 20 nt, Artificial Sequence
  Synthetic Construct
cctcgtaggtcttgatgggc

SEQ ID NO: 210 - RNA, 20 nt, Artificial Sequence
  Synthetic Construct
gctgcagtgtcacattgccc

SEQ ID NO: 211 - RNA, 20 nt, Artificial Sequence
  Synthetic Construct
acgggcatctggaagagcgc

SEQ ID NO: 212 - RNA, 20 nt, Artificial Sequence
  Synthetic Construct
gggccttgttgaccacgtcc

SEQ ID NO: 213 - RNA, 20 nt, Artificial Sequence
  Synthetic Construct
agcttggtgggcaggtgtgg

SEQ ID NO: 214 - RNA, 20 nt, Artificial Sequence
  Synthetic Construct
gcttgacagtgatgacctcc

SEQ ID NO: 215 - RNA, 20 nt, Artificial Sequence
  Synthetic Construct
gccttgttgaccacgtcctg

SEQ ID NO: 216 - RNA, 20 nt, Artificial Sequence
  Synthetic Construct
accaggtggagggttggagg

SEQ ID NO: 217 - RNA, 20 nt, Artificial Sequence
  Synthetic Construct
gtcctcgtcggcgtccaagg

SEQ ID NO: 218 - RNA, 20 nt, Artificial Sequence
  Synthetic Construct
accaagcttgtgatccggca

SEQ ID NO: 219 - RNA, 20 nt, Artificial Sequence
  Synthetic Construct
atgacctcctggataatccc

SEQ ID NO: 220 - RNA, 20 nt, Artificial Sequence
  Synthetic Construct
tcatcctccagaacaaggct

SEQ ID NO: 221 - RNA, 20 nt, Artificial Sequence
  Synthetic Construct
ggtgcgtttcagcagctgcg

SEQ ID NO: 222 - RNA, 20 nt, Artificial Sequence
  Synthetic Construct
tgctgctgccttggttcagg

SEQ ID NO: 223 - RNA, 20 nt, Artificial Sequence
  Synthetic Construct
gcgatgcagggcgtcctcaa

SEQ ID NO: 224 - RNA, 20 nt, Artificial Sequence
  Synthetic Construct
ggtcatccagaagaacctct

SEQ ID NO: 225 - RNA, 20 nt, Artificial Sequence
  Synthetic Construct
cttggccagctgtttatcca

SEQ ID NO: 226 - RNA, 20 nt, Artificial Sequence
  Synthetic Construct
cggggcgctgactatgaccg

SEQ ID NO: 227 - RNA, 20 nt, Artificial Sequence
  Synthetic Construct
cctcctctgagacgtcctcc

SEQ ID NO: 228 - RNA, 20 nt, Artificial Sequence
  Synthetic Construct
aagccgatgaccctcggaag

SEQ ID NO: 229 - RNA, 20 nt, Artificial Sequence
  Synthetic Construct
tctaggtccacactgggggc

SEQ ID NO: 230 - RNA, 20 nt, Artificial Sequence
  Synthetic Construct
tcggtgagcgtgtcatcctg

SEQ ID NO: 231 - RNA, 20 nt, Artificial Sequence
  Synthetic Construct
cccatcgcagcctcttccga

SEQ ID NO: 232 - RNA, 20 nt, Artificial Sequence
  Synthetic Construct
gtgacactgcagcccatccc

SEQ ID NO: 233 - RNA, 20 nt, Artificial Sequence
  Synthetic Construct
caagtccgagtcgcccgacg

SEQ ID NO: 234 - RNA, 20 nt, Artificial Sequence
  Synthetic Construct
tccccctccaaccctccacc

SEQ ID NO: 235 - RNA, 20 nt, Artificial Sequence
  Synthetic Construct
gctccgtgatgttggcctct

SEQ ID NO: 236 - RNA, 20 nt, Artificial Sequence
  Synthetic Construct
gctggtggcctcgtccactt

SEQ ID NO: 237 - RNA, 20 nt, Artificial Sequence
  Synthetic Construct
accatctggaaggtccgagg

SEQ ID NO: 238 - RNA, 20 nt, Artificial Sequence
  Synthetic Construct
ccgggcctcgtaggtcttga

SEQ ID NO: 239 - RNA, 20 nt, Artificial Sequence
  Synthetic Construct
gacctaccgcgagaacgtgg

SEQ ID NO: 240 - RNA, 20 nt, Artificial Sequence
  Synthetic Construct
cctccccgggattatccagg

SEQ ID NO: 241 - RNA, 20 nt, Artificial Sequence
  Synthetic Construct
ggagaggatgtgcgcgctgt

SEQ ID NO: 242 - RNA, 20 nt, Artificial Sequence
  Synthetic Construct
gggtgcaagggtggctctga

SEQ ID NO: 243 - RNA, 20 nt, Artificial Sequence
  Synthetic Construct
tgagctgtccacctgtgtgg

SEQ ID NO: 244 - RNA, 20 nt, Artificial Sequence
  Synthetic Construct
gcagctgctgaaacgcaccc

SEQ ID NO: 245 - RNA, 20 nt, Artificial Sequence
  Synthetic Construct
agcccggagaagatcgtcct

SEQ ID NO: 246 - RNA, 20 nt, Artificial Sequence
  Synthetic Construct
tcagtgggcaggccccatct

SEQ ID NO: 247 - RNA, 20 nt, Artificial Sequence
  Synthetic Construct
accttcatagaaggcacttt

SEQ ID NO: 248 - RNA, 20 nt, Artificial Sequence
  Synthetic Construct
cctccaagaggccaacatca

SEQ ID NO: 249 - RNA, 20 nt, Artificial Sequence
  Synthetic Construct
acccaggtccagctcagcct

SEQ ID NO: 250 - RNA, 20 nt, Artificial Sequence
  Synthetic Construct
tccttgctgggctgaagcgt

SEQ ID NO: 251 - RNA, 20 nt, Artificial Sequence
  Synthetic Construct
tgtcatcttccaggaagtct

SEQ ID NO: 252 - RNA, 20 nt, Artificial Sequence
  Synthetic Construct
tttgtcatccaaaaccagct

SEQ ID NO: 253 - RNA, 20 nt, Artificial Sequence
  Synthetic Construct
aggcccacaggctgcaggaa

SEQ ID NO: 254 - RNA, 20 nt, Artificial Sequence
  Synthetic Construct
agcccgacagcaccacgttc

SEQ ID NO: 255 - RNA, 20 nt, Artificial Sequence
  Synthetic Construct
gctgtggccaccacgctcaa

SEQ ID NO: 256 - RNA, 20 nt, Artificial Sequence
  Synthetic Construct
caggatctgcccgcccacgt

SEQ ID NO: 257 - RNA, 20 nt, Artificial Sequence
  Synthetic Construct
ggtagacatggtagggcagg

SEQ ID NO: 258 - RNA, 20 nt, Artificial Sequence
  Synthetic Construct
caacgtgggcgggcagatcc

SEQ ID NO: 259 - RNA, 20 nt, Artificial Sequence
  Synthetic Construct
ccttgccttggataaacagc

SEQ ID NO: 260 - RNA, 20 nt, Artificial Sequence
  Synthetic Construct
tcgagaagagccttcggctg

SEQ ID NO: 261 - RNA, 20 nt, Artificial Sequence
  Synthetic Construct
ccttgctgggctgaagcgtg

SEQ ID NO: 262 - RNA, 20 nt, Artificial Sequence
  Synthetic Construct
ggcctggtgaggtagtgacg

SEQ ID NO: 263 - RNA, 20 nt, Artificial Sequence
  Synthetic Construct
tctcgagaagagccttcggc

SEQ ID NO: 264 - RNA, 20 nt, Artificial Sequence
  Synthetic Construct
attgagcgtggtggccacag

SEQ ID NO: 265 - RNA, 20 nt, Artificial Sequence
  Synthetic Construct
ctcgtcggcgtccaaggagg

SEQ ID NO: 266 - RNA, 20 nt, Artificial Sequence
  Synthetic Construct
gctggcaaaagccttgttct

SEQ ID NO: 267 - RNA, 20 nt, Artificial Sequence
  Synthetic Construct
caggaagggctgcacactca

SEQ ID NO: 268 - RNA, 20 nt, Artificial Sequence
  Synthetic Construct
gatgagctgtccacctgtgt

SEQ ID NO: 269 - RNA, 20 nt, Artificial Sequence
  Synthetic Construct
caacatcacggagcagacgc

SEQ ID NO: 270 - RNA, 20 nt, Artificial Sequence
  Synthetic Construct
caggcccacaggctgcagga

SEQ ID NO: 271 - RNA, 20 nt, Artificial Sequence
  Synthetic Construct
tgcagggtgggcagctggaa

SEQ ID NO: 272 - RNA, 20 nt, Artificial Sequence
  Synthetic Construct
cctgccctaccatgtctacc

SEQ ID NO: 273 - RNA, 20 nt, Artificial Sequence
  Synthetic Construct
aaagtggacgaggccaccag

SEQ ID NO: 274 - RNA, 20 nt, Artificial Sequence
  Synthetic Construct
ctcaatgggaactctgtgtt

SEQ ID NO: 275 - RNA, 20 nt, Artificial Sequence
  Synthetic Construct
tcgcggtaggtcttgcgcag

SEQ ID NO: 276 - RNA, 20 nt, Artificial Sequence
  Synthetic Construct
ggaaggccgtcttgtagtcg

SEQ ID NO: 277 - RNA, 20 nt, Artificial Sequence
  Synthetic Construct
gccttggccacattcagggt

SEQ ID NO: 278 - RNA, 20 nt, Artificial Sequence
  Synthetic Construct
gcagaacctgacgttcatgg

SEQ ID NO: 279 - RNA, 20 nt, Artificial Sequence
  Synthetic Construct
gtcatcctgcgggctgtcgc

SEQ ID NO: 280 - RNA, 20 nt, Artificial Sequence
  Synthetic Construct
cctcacaaaccagaacctgg

SEQ ID NO: 281 - RNA, 20 nt, Artificial Sequence
  Synthetic Construct
aacacggggcccaggctcag

SEQ ID NO: 282 - RNA, 20 nt, Artificial Sequence
  Synthetic Construct
catcctcacaaaccagaacc

SEQ ID NO: 283 - RNA, 20 nt, Artificial Sequence
  Synthetic Construct
ctccatgtgcaagaagcttc

SEQ ID NO: 284 - RNA, 20 nt, Artificial Sequence
  Synthetic Construct
agggggagtgggggacttgt

SEQ ID NO: 285 - RNA, 20 nt, Artificial Sequence
  Synthetic Construct
gcgctgactatgaccgaggc

SEQ ID NO: 286 - RNA, 20 nt, Artificial Sequence
  Synthetic Construct
aagcggccttggccacattc

SEQ ID NO: 287 - RNA, 20 nt, Artificial Sequence
  Synthetic Construct
ttcaggaggtggacgctcat

SEQ ID NO: 288 - RNA, 20 nt, Artificial Sequence
  Synthetic Construct
ttgttctcggctggaagcag

SEQ ID NO: 289 - RNA, 20 nt, Artificial Sequence
  Synthetic Construct
cttctccagcgacagcccgc

SEQ ID NO: 290 - RNA, 20 nt, Artificial Sequence
  Synthetic Construct
cctggtagacatggtagggc

SEQ ID NO: 291 - RNA, 20 nt, Artificial Sequence
  Synthetic Construct
gctggaggatgtcacagggc

SEQ ID NO: 292 - RNA, 20 nt, Artificial Sequence
  Synthetic Construct
ggcgccctggtagacatggt

SEQ ID NO: 293 - RNA, 20 nt, Artificial Sequence
  Synthetic Construct
gatgccaggcagagttgggg

SEQ ID NO: 294 - RNA, 20 nt, Artificial Sequence
  Synthetic Construct
ggtccaccgtgccgttcatc

SEQ ID NO: 295 - RNA, 20 nt, Artificial Sequence
  Synthetic Construct
ccttggccacattcagggtc

SEQ ID NO: 296 - RNA, 20 nt, Artificial Sequence
  Synthetic Construct
gccatgctcaacacccccga

SEQ ID NO: 297 - RNA, 20 nt, Artificial Sequence
  Synthetic Construct
tgctgtcgatggcgctctgc

SEQ ID NO: 298 - RNA, 20 nt, Artificial Sequence
  Synthetic Construct
gcctcgtaggtcttgatggg

SEQ ID NO: 299 - RNA, 20 nt, Artificial Sequence
  Synthetic Construct
gcaggaagggctgcacactc

SEQ ID NO: 300 - RNA, 20 nt, Artificial Sequence
  Synthetic Construct
ctggaagtcggatggcgtag

SEQ ID NO: 301 - RNA, 20 nt, Artificial Sequence
  Synthetic Construct
gtagggcaggaggcgatgca

SEQ ID NO: 302 - RNA, 20 nt, Artificial Sequence
  Synthetic Construct
tggcgtaggggctggcaacc

SEQ ID NO: 303 - RNA, 20 nt, Artificial Sequence
  Synthetic Construct
gctctgctggaggatgtcac

SEQ ID NO: 304 - RNA, 20 nt, Artificial Sequence
  Synthetic Construct
tctggcccggcagcatgtgc

SEQ ID NO: 305 - RNA, 20 nt, Artificial Sequence
  Synthetic Construct
gagggggagtgggggacttg

SEQ ID NO: 306 - RNA, 20 nt, Artificial Sequence
  Synthetic Construct
aggtagtgacggggacgaag

SEQ ID NO: 307 - RNA, 20 nt, Artificial Sequence
  Synthetic Construct
caacctcccgaccctgaatg

SEQ ID NO: 308 - RNA, 20 nt, Artificial Sequence
  Synthetic Construct
gctggttgggctctgggttc

SEQ ID NO: 309 - RNA, 20 nt, Artificial Sequence
  Synthetic Construct
gggccagaacgtggtgctgt

SEQ ID NO: 310 - RNA, 20 nt, Artificial Sequence
  Synthetic Construct
caccgttgtgactggagggt

SEQ ID NO: 311 - RNA, 20 nt, Artificial Sequence
  Synthetic Construct
tctcctcctgaatgaacatt

SEQ ID NO: 312 - RNA, 20 nt, Artificial Sequence
  Synthetic Construct
gcagcccttcctgcagcctg

SEQ ID NO: 313 - RNA, 20 nt, Artificial Sequence
  Synthetic Construct
tccttggacgccgacgagga

SEQ ID NO: 314 - RNA, 20 nt, Artificial Sequence
  Synthetic Construct
gaccaggtggagggttggag

SEQ ID NO: 315 - RNA, 20 nt, Artificial Sequence
  Synthetic Construct
caacctggaggacgtctcag

SEQ ID NO: 316 - RNA, 20 nt, Artificial Sequence
  Synthetic Construct
ccgtgatgttggcctcttgg

SEQ ID NO: 317 - RNA, 20 nt, Artificial Sequence
  Synthetic Construct
tcctgagtgaggatggccgt

SEQ ID NO: 318 - RNA, 20 nt, Artificial Sequence
  Synthetic Construct
ggacacactctgtggccggg

SEQ ID NO: 319 - RNA, 20 nt, Artificial Sequence
  Synthetic Construct
actgaccagcacagacgcag

SEQ ID NO: 320 - RNA, 20 nt, Artificial Sequence
  Synthetic Construct
tcctccagaacaaggctggg

SEQ ID NO: 321 - RNA, 20 nt, Artificial Sequence
  Synthetic Construct
ggagcatttgcacaaacacc

SEQ ID NO: 322 - RNA, 20 nt, Artificial Sequence
  Synthetic Construct
atcatcctccagaacaaggc

SEQ ID NO: 323 - RNA, 20 nt, Artificial Sequence
  Synthetic Construct
ggccttgttgaccacgtcct

SEQ ID NO: 324 - RNA, 20 nt, Artificial Sequence
  Synthetic Construct
gacacactctgtggccggga

SEQ ID NO: 325 - RNA, 20 nt, Artificial Sequence
  Synthetic Construct
gggccagatgaacggcacgg

SEQ ID NO: 326 - RNA, 20 nt, Artificial Sequence
  Synthetic Construct
aggttctggtttgtgaggat

SEQ ID NO: 327 - RNA, 20 nt, Artificial Sequence
  Synthetic Construct
aatgggaactctgtgttcgg

SEQ ID NO: 328 - RNA, 20 nt, Artificial Sequence
  Synthetic Construct
gctcaagctcaagatcaagc

SEQ ID NO: 329 - RNA, 20 nt, Artificial Sequence
  Synthetic Construct
gcacctgcttggccaggagc

SEQ ID NO: 330 - RNA, 20 nt, Artificial Sequence
  Synthetic Construct
ttgtggtagtcactggggga

SEQ ID NO: 331 - RNA, 20 nt, Artificial Sequence
  Synthetic Construct
gaagcgtggggggcttcttc

SEQ ID NO: 332 - RNA, 20 nt, Artificial Sequence
  Synthetic Construct
gtgccgttcatctggcccgt

SEQ ID NO: 333 - RNA, 20 nt, Artificial Sequence
  Synthetic Construct
cagcggccgagaggttcttc

SEQ ID NO: 334 - RNA, 20 nt, Artificial Sequence
  Synthetic Construct
gactatgaccgaggcaggca

SEQ ID NO: 335 - RNA, 20 nt, Artificial Sequence
  Synthetic Construct
agctcctttggcgacggggg

SEQ ID NO: 336 - RNA, 20 nt, Artificial Sequence
  Synthetic Construct
ggacgctcatgggtttgctc

SEQ ID NO: 337 - RNA, 20 nt, Artificial Sequence
  Synthetic Construct
ccgtcttgtagtcggggtgc

SEQ ID NO: 338 - RNA, 20 nt, Artificial Sequence
  Synthetic Construct
ccccatcgcagcctcttccg

SEQ ID NO: 339 - RNA, 20 nt, Artificial Sequence
  Synthetic Construct
tgtgacactgcagcccatcc

SEQ ID NO: 340 - RNA, 20 nt, Artificial Sequence
  Synthetic Construct
ggggaaggccgtcttgtagt

SEQ ID NO: 341 - RNA, 20 nt, Artificial Sequence
  Synthetic Construct
cccgcagaacctgacgttca

SEQ ID NO: 342 - RNA, 20 nt, Artificial Sequence
  Synthetic Construct
catcacggagcagacgctgg

SEQ ID NO: 343 - RNA, 20 nt, Artificial Sequence
  Synthetic Construct
aagctggcctggtccacgtc

SEQ ID NO: 344 - RNA, 20 nt, Artificial Sequence
  Synthetic Construct
tgtggtagtcactgggggag

SEQ ID NO: 345 - RNA, 20 nt, Artificial Sequence
  Synthetic Construct
gctgcgtctgtgctggtcag

SEQ ID NO: 346 - RNA, 20 nt, Artificial Sequence
  Synthetic Construct
ggtgtttgtgcaaatgctcc

SEQ ID NO: 347 - RNA, 20 nt, Artificial Sequence
  Synthetic Construct
ggaagtctaggtccacactg

SEQ ID NO: 348 - RNA, 20 nt, Artificial Sequence
  Synthetic Construct
gaacctgacgttcatggcgg

SEQ ID NO: 349 - RNA, 20 nt, Artificial Sequence
  Synthetic Construct
gtagtgacggggacgaagag

SEQ ID NO: 350 - RNA, 20 nt, Artificial Sequence
  Synthetic Construct
gacgctcatgggtttgctca

SEQ ID NO: 351 - RNA, 20 nt, Artificial Sequence
  Synthetic Construct
cctccagaacaaggctgggg

SEQ ID NO: 352 - RNA, 20 nt, Artificial Sequence
  Synthetic Construct
cggaatcatcctccagaaca

SEQ ID NO: 353 - RNA, 20 nt, Artificial Sequence
  Synthetic Construct
gcctggtgggtcagcacggt

SEQ ID NO: 354 - RNA, 20 nt, Artificial Sequence
  Synthetic Construct
ggcgtgttgagcgccatgac

SEQ ID NO: 355 - RNA, 20 nt, Artificial Sequence
  Synthetic Construct
gttcaggaggtggacgctca

SEQ ID NO: 356 - RNA, 20 nt, Artificial Sequence
  Synthetic Construct
tccgagggtcatcggcttcc

SEQ ID NO: 357 - RNA, 20 nt, Artificial Sequence
  Synthetic Construct
cggaagaggctgcgatgggg

SEQ ID NO: 358 - RNA, 20 nt, Artificial Sequence
  Synthetic Construct
tggccacattcagggtcggg

SEQ ID NO: 359 - RNA, 20 nt, Artificial Sequence
  Synthetic Construct
ctgcgtctgtgctggtcagt

SEQ ID NO: 360 - RNA, 20 nt, Artificial Sequence
  Synthetic Construct
ctgcttggccaggagctggg

SEQ ID NO: 361 - RNA, 20 nt, Artificial Sequence
  Synthetic Construct
ggtgagcccctcagcggaga

SEQ ID NO: 362 - RNA, 20 nt, Artificial Sequence
  Synthetic Construct
cacgggccagatgaacggca

SEQ ID NO: 363 - RNA, 20 nt, Artificial Sequence
  Synthetic Construct
aactctgtgttcggaggcgc

SEQ ID NO: 364 - RNA, 20 nt, Artificial Sequence
  Synthetic Construct
tgctgccttggttcaggagg

SEQ ID NO: 365 - RNA, 20 nt, Artificial Sequence
  Synthetic Construct
ggagggcgccctggtagaca

SEQ ID NO: 366 - RNA, 20 nt, Artificial Sequence
  Synthetic Construct
gccatgaacgtcaggttctg

SEQ ID NO: 367 - RNA, 20 nt, Artificial Sequence
  Synthetic Construct
ttgttgaccacgtcctgggg

SEQ ID NO: 368 - RNA, 20 nt, Artificial Sequence
  Synthetic Construct
taaatatcggctcctgctcc

SEQ ID NO: 369 - RNA, 20 nt, Artificial Sequence
  Synthetic Construct
cccgaccctgaatgtggcca

SEQ ID NO: 370 - RNA, 20 nt, Artificial Sequence
  Synthetic Construct
ccctcggaagaggctgcgat

SEQ ID NO: 371 - RNA, 20 nt, Artificial Sequence
  Synthetic Construct
tacccaccctccagtcacaa

SEQ ID NO: 372 - RNA, 20 nt, Artificial Sequence
  Synthetic Construct
accccccgctaccctcaagg

SEQ ID NO: 373 - RNA, 20 nt, Artificial Sequence
  Synthetic Construct
gggggcccttcagcatacgc

SEQ ID NO: 374 - RNA, 20 nt, Artificial Sequence
  Synthetic Construct
gcccaaagtgccttctatga

SEQ ID NO: 375 - RNA, 20 nt, Artificial Sequence
  Synthetic Construct
tcagggaccaggtggagggt

SEQ ID NO: 376 - RNA, 20 nt, Artificial Sequence
  Synthetic Construct
gtcagctggtagagcttggg

SEQ ID NO: 377 - RNA, 20 nt, Artificial Sequence
  Synthetic Construct
ctgcctgggaagagggcttg

SEQ ID NO: 378 - RNA, 20 nt, Artificial Sequence
  Synthetic Construct
tgctcctggaggagtcccgg

SEQ ID NO: 379 - RNA, 20 nt, Artificial Sequence
  Synthetic Construct
caggcagtaggaactggttc

SEQ ID NO: 380 - RNA, 20 nt, Artificial Sequence
  Synthetic Construct
gatggcgctctgcaggtgct

SEQ ID NO: 381 - RNA, 20 nt, Artificial Sequence
  Synthetic Construct
cttcctggaagatgacatcc

SEQ ID NO: 382 - RNA, 20 nt, Artificial Sequence
  Synthetic Construct
acagctggccaaggagaagc

SEQ ID NO: 383 - RNA, 20 nt, Artificial Sequence
  Synthetic Construct
ctgaccagcacagacgcagc

SEQ ID NO: 384 - RNA, 20 nt, Artificial Sequence
  Synthetic Construct
gaagtctaggtccacactgg

SEQ ID NO: 385 - RNA, 20 nt, Artificial Sequence
  Synthetic Construct
atgggtttgctcagggcccc

SEQ ID NO: 386 - RNA, 20 nt, Artificial Sequence
  Synthetic Construct
gatgacctcctggataatcc

SEQ ID NO: 387 - RNA, 20 nt, Artificial Sequence
  Synthetic Construct
gtgccttctatgaaggtcct

SEQ ID NO: 388 - RNA, 20 nt, Artificial Sequence
  Synthetic Construct
ccccacggccatcctcactc

SEQ ID NO: 389 - RNA, 20 nt, Artificial Sequence
  Synthetic Construct
agcttgtgatccggcacggc

SEQ ID NO: 390 - RNA, 20 nt, Artificial Sequence
  Synthetic Construct
cctcctggataatcccgggg

SEQ ID NO: 391 - RNA, 20 nt, Artificial Sequence
  Synthetic Construct
ctacctcaccaggcccctct

SEQ ID NO: 392 - RNA, 20 nt, Artificial Sequence
  Synthetic Construct
ctctgggttcaggtggttgc

SEQ ID NO: 393 - RNA, 20 nt, Artificial Sequence
  Synthetic Construct
tgcaagggtggctctgaggg

SEQ ID NO: 394 - RNA, 20 nt, Artificial Sequence
  Synthetic Construct
caaacggcttgggcgtcagc

SEQ ID NO: 395 - RNA, 20 nt, Artificial Sequence
  Synthetic Construct
ttgctgggctgaagcgtggg

SEQ ID NO: 396 - RNA, 20 nt, Artificial Sequence
  Synthetic Construct
tggtcaacgtcctggcgccg

SEQ ID NO: 397 - RNA, 20 nt, Artificial Sequence
  Synthetic Construct
ggcgtcagctggtagagctt

SEQ ID NO: 398 - RNA, 20 nt, Artificial Sequence
  Synthetic Construct
gtgccggatcacaagcttgg

SEQ ID NO: 399 - RNA, 20 nt, Artificial Sequence
  Synthetic Construct
tgcagggcgtcctcaaagga

SEQ ID NO: 400 - RNA, 20 nt, Artificial Sequence
  Synthetic Construct
gatcgaccgaatgttcattc

SEQ ID NO: 401 - RNA, 20 nt, Artificial Sequence
  Synthetic Construct
cctggggcaggaacatctgc

SEQ ID NO: 402 - RNA, 20 nt, Artificial Sequence
  Synthetic Construct
cagcgtctgctccgtgatgt

SEQ ID NO: 403 - RNA, 20 nt, Artificial Sequence
  Synthetic Construct
tccatgcaagaagtcattga

SEQ ID NO: 404 - RNA, 20 nt, Artificial Sequence
  Synthetic Construct
caggaagtctaggtccacac

SEQ ID NO: 405 - RNA, 20 nt, Artificial Sequence
  Synthetic Construct
ccccagtgactaccacaaag

SEQ ID NO: 406 - RNA, 20 nt, Artificial Sequence
  Synthetic Construct
tggcctcttggaggctctgc

SEQ ID NO: 407 - RNA, 20 nt, Artificial Sequence
  Synthetic Construct
ttccttgctgggctgaagcg

SEQ ID NO: 408 - RNA, 20 nt, Artificial Sequence
  Synthetic Construct
gggggtggtcaccatctgga

SEQ ID NO: 409 - RNA, 20 nt, Artificial Sequence
  Synthetic Construct
cagaaccagttcctactgcc

SEQ ID NO: 410 - RNA, 20 nt, Artificial Sequence
  Synthetic Construct
ctgtggccaccacgctcaat

SEQ ID NO: 411 - RNA, 20 nt, Artificial Sequence
  Synthetic Construct
cagcaaggtcgtgcacaaca

SEQ ID NO: 412 - RNA, 20 nt, Artificial Sequence
  Synthetic Construct
gacgttcatggcggcgggga

SEQ ID NO: 413 - RNA, 20 nt, Artificial Sequence
  Synthetic Construct
agtggacgaggagtttgaga

SEQ ID NO: 414 - RNA, 20 nt, Artificial Sequence
  Synthetic Construct
gggaccaggtggagggttgg

SEQ ID NO: 415 - RNA, 20 nt, Artificial Sequence
  Synthetic Construct
ctgtaccagcgtatgctgaa

SEQ ID NO: 416 - RNA, 20 nt, Artificial Sequence
  Synthetic Construct
tcagtgggcccgccaggttc

SEQ ID NO: 417 - RNA, 20 nt, Artificial Sequence
  Synthetic Construct
gggcgccaggcagtaggaac

SEQ ID NO: 418 - RNA, 20 nt, Artificial Sequence
  Synthetic Construct
gggggtgttgagcatggcca

SEQ ID NO: 419 - RNA, 20 nt, Artificial Sequence
  Synthetic Construct
gaccttcatagaaggcactt

SEQ ID NO: 420 - RNA, 20 nt, Artificial Sequence
  Synthetic Construct
tctcggctggaagcagagga

SEQ ID NO: 421 - RNA, 20 nt, Artificial Sequence
  Synthetic Construct
gtctgtgctggtcagtgggc

SEQ ID NO: 422 - RNA, 20 nt, Artificial Sequence
  Synthetic Construct
gctggaagtcggatggcgta

SEQ ID NO: 423 - RNA, 20 nt, Artificial Sequence
  Synthetic Construct
gttgagcatggccacggcgc

SEQ ID NO: 424 - RNA, 20 nt, Artificial Sequence
  Synthetic Construct
aagcttgtgatccggcacgg

SEQ ID NO: 425 - RNA, 20 nt, Artificial Sequence
  Synthetic Construct
ggtagggcaggaggcgatgc

SEQ ID NO: 426 - RNA, 20 nt, Artificial Sequence
  Synthetic Construct
gtcggtgctgcctgggaaga

SEQ ID NO: 427 - RNA, 20 nt, Artificial Sequence
  Synthetic Construct
catgggtttgctcagggccc

SEQ ID NO: 428 - RNA, 20 nt, Artificial Sequence
  Synthetic Construct
tgggaactggagctggaagt

SEQ ID NO: 429 - RNA, 20 nt, Artificial Sequence
  Synthetic Construct
gaggccagctcctttggcga

SEQ ID NO: 430 - RNA, 20 nt, Artificial Sequence
  Synthetic Construct
gtcaccatctggaaggtccg

SEQ ID NO: 431 - RNA, 20 nt, Artificial Sequence
  Synthetic Construct
aatctctcgagaagagcctt

SEQ ID NO: 432 - RNA, 20 nt, Artificial Sequence
  Synthetic Construct
agtgaggatggccgtggggg

SEQ ID NO: 433 - RNA, 20 nt, Artificial Sequence
  Synthetic Construct
aagatgccaggcagagttgg

SEQ ID NO: 434 - RNA, 20 nt, Artificial Sequence
  Synthetic Construct
cctgcagatgttcctgcccc

SEQ ID NO: 435 - RNA, 20 nt, Artificial Sequence
  Synthetic Construct
gcaggaagccgggctcagca

SEQ ID NO: 436 - RNA, 20 nt, Artificial Sequence
  Synthetic Construct
cgggcctcgtaggtcttgat

SEQ ID NO: 437 - RNA, 20 nt, Artificial Sequence
  Synthetic Construct
gtcctgagtgaggatggccg

SEQ ID NO: 438 - RNA, 20 nt, Artificial Sequence
  Synthetic Construct
acctaccgcgagaacgtggg

SEQ ID NO: 439 - RNA, 20 nt, Artificial Sequence
  Synthetic Construct
ctccagttcccacccagcca

SEQ ID NO: 440 - RNA, 20 nt, Artificial Sequence
  Synthetic Construct
aactggagctggaagtcgga

SEQ ID NO: 441 - RNA, 20 nt, Artificial Sequence
  Synthetic Construct
gggaaggccgtcttgtagtc

SEQ ID NO: 442 - RNA, 20 nt, Artificial Sequence
  Synthetic Construct
ggggcctggtgaggtagtga

SEQ ID NO: 443 - RNA, 20 nt, Artificial Sequence
  Synthetic Construct
acctgacgttcatggcggcg

SEQ ID NO: 444 - RNA, 20 nt, Artificial Sequence
  Synthetic Construct
gccgtcgggggtgttgagca

SEQ ID NO: 445 - RNA, 20 nt, Artificial Sequence
  Synthetic Construct
ggtagtcactgggggagggg

SEQ ID NO: 446 - RNA, 20 nt, Artificial Sequence
  Synthetic Construct
ctcctccttggacgccgacg

SEQ ID NO: 447 - RNA, 20 nt, Artificial Sequence
  Synthetic Construct
actctgtgttcggaggcgcg

SEQ ID NO: 448 - RNA, 20 nt, Artificial Sequence
  Synthetic Construct
cgtaggggctggcaacctgg

SEQ ID NO: 449 - RNA, 20 nt, Artificial Sequence
  Synthetic Construct
cttgtagtcggggtgcagga

SEQ ID NO: 450 - RNA, 20 nt, Artificial Sequence
  Synthetic Construct
cgtgccgttcatctggcccg

SEQ ID NO: 451 - RNA, 20 nt, Artificial Sequence
  Synthetic Construct
atgaccaggccagccccctg

SEQ ID NO: 452 - RNA, 20 nt, Artificial Sequence
  Synthetic Construct
gtccccgtcactacctcacc

SEQ ID NO: 453 - RNA, 20 nt, Artificial Sequence
  Synthetic Construct
agaagtcattgagggcctgt

SEQ ID NO: 454 - RNA, 20 nt, Artificial Sequence
  Synthetic Construct
agcccaggatgtcatcttcc

SEQ ID NO: 455 - RNA, 20 nt, Artificial Sequence
  Synthetic Construct
ctgagagctgctgcgggagc

SEQ ID NO: 456 - RNA, 20 nt, Artificial Sequence
  Synthetic Construct
agctggaagtcggatggcgt

SEQ ID NO: 457 - RNA, 20 nt, Artificial Sequence
  Synthetic Construct
gtggcctcgtccactttggc

SEQ ID NO: 458 - RNA, 20 nt, Artificial Sequence
  Synthetic Construct
ggtcggtgctgcctgggaag

SEQ ID NO: 459 - RNA, 20 nt, Artificial Sequence
  Synthetic Construct
ggttctggtttgtgaggatg

SEQ ID NO: 460 - RNA, 20 nt, Artificial Sequence
  Synthetic Construct
actggagggtgggtaggcct

SEQ ID NO: 461 - RNA, 20 nt, Artificial Sequence
  Synthetic Construct
gaacccagagcccaaccagc

SEQ ID NO: 462 - RNA, 20 nt, Artificial Sequence
  Synthetic Construct
cctgagtgaggatggccgtg

SEQ ID NO: 463 - RNA, 20 nt, Artificial Sequence
  Synthetic Construct
attcagggtcgggaggttgc

SEQ ID NO: 464 - RNA, 20 nt, Artificial Sequence
  Synthetic Construct
gcctaccgtgctgacccacc

SEQ ID NO: 465 - RNA, 20 nt, Artificial Sequence
  Synthetic Construct
cagtatgagagcaaactgag

SEQ ID NO: 466 - RNA, 20 nt, Artificial Sequence
  Synthetic Construct
aggccatgctcaataaatat

SEQ ID NO: 467 - RNA, 20 nt, Artificial Sequence
  Synthetic Construct
gggcctggtgaggtagtgac

SEQ ID NO: 468 - RNA, 20 nt, Artificial Sequence
  Synthetic Construct
cagcatcctgaacctgcagc

SEQ ID NO: 469 - RNA, 20 nt, Artificial Sequence
  Synthetic Construct
gccccaggacgtggtcaaca

SEQ ID NO: 470 - RNA, 20 nt, Artificial Sequence
  Synthetic Construct
atgagctgtccacctgtgtg

SEQ ID NO: 471 - RNA, 20 nt, Artificial Sequence
  Synthetic Construct
tcttgatgggcgggcggttg

SEQ ID NO: 472 - RNA, 20 nt, Artificial Sequence
  Synthetic Construct
cagcctcttccgagggtcat

SEQ ID NO: 473 - RNA, 20 nt, Artificial Sequence
  Synthetic Construct
ccatgaacgtcaggttctgc

SEQ ID NO: 474 - RNA, 20 nt, Artificial Sequence
  Synthetic Construct
aagtcggatggcgtaggggc

SEQ ID NO: 475 - RNA, 20 nt, Artificial Sequence
  Synthetic Construct
cgatgctgctgccttggttc

SEQ ID NO: 476 - RNA, 20 nt, Artificial Sequence
  Synthetic Construct
tgggcgtgggtgtgcgatgc

SEQ ID NO: 477 - RNA, 20 nt, Artificial Sequence
  Synthetic Construct
ggtaggtcttgcgcagtggc

SEQ ID NO: 478 - RNA, 20 nt, Artificial Sequence
  Synthetic Construct
ggatcacaagcttggtgggc

SEQ ID NO: 479 - RNA, 20 nt, Artificial Sequence
  Synthetic Construct
ctggtacagctcgtcctcca

SEQ ID NO: 480 - RNA, 20 nt, Artificial Sequence
  Synthetic Construct
ccccacgcttcagcccagca

SEQ ID NO: 481 - RNA, 20 nt, Artificial Sequence
  Synthetic Construct
accttgagggtagcgggggg

SEQ ID NO: 482 - RNA, 20 nt, Artificial Sequence
  Synthetic Construct
acaaagatgccaggcagagt

SEQ ID NO: 483 - RNA, 20 nt, Artificial Sequence
  Synthetic Construct
gacggccttcccctcctttg

SEQ ID NO: 484 - RNA, 20 nt, Artificial Sequence
  Synthetic Construct
cttgctgggctgaagcgtgg

SEQ ID NO: 485 - RNA, 20 nt, Artificial Sequence
  Synthetic Construct
tcttctccgggctagacgcc

SEQ ID NO: 486 - RNA, 20 nt, Artificial Sequence
  Synthetic Construct
aggccagctcctttggcgac

SEQ ID NO: 487 - RNA, 20 nt, Artificial Sequence
  Synthetic Construct
ggcgtcctcaaaggagggga

SEQ ID NO: 488 - RNA, 20 nt, Artificial Sequence
  Synthetic Construct
aggaagtctaggtccacact

SEQ ID NO: 489 - RNA, 20 nt, Artificial Sequence
  Synthetic Construct
ggagggcgggacacactctg

SEQ ID NO: 490 - RNA, 20 nt, Artificial Sequence
  Synthetic Construct
cagtgtggacctagacttcc

SEQ ID NO: 491 - RNA, 20 nt, Artificial Sequence
  Synthetic Construct
gtcacattgcccaggcccac

SEQ ID NO: 492 - RNA, 20 nt, Artificial Sequence
  Synthetic Construct
gcttggtgggcaggtgtgga

SEQ ID NO: 493 - RNA, 20 nt, Artificial Sequence
  Synthetic Construct
gccctcaatgacttcttgca

SEQ ID NO: 494 - RNA, 20 nt, Artificial Sequence
  Synthetic Construct
ccaccgttgtgactggaggg

SEQ ID NO: 495 - RNA, 20 nt, Artificial Sequence
  Synthetic Construct
tgaccagcacagacgcagcg

SEQ ID NO: 496 - RNA, 20 nt, Artificial Sequence
  Synthetic Construct
cagctgtttatccaaggcaa

SEQ ID NO: 497 - RNA, 20 nt, Artificial Sequence
  Synthetic Construct
tgacgggcacctgcttggcc

SEQ ID NO: 498 - RNA, 20 nt, Artificial Sequence
  Synthetic Construct
cacagagttcccattgagcg

SEQ ID NO: 499 - RNA, 20 nt, Artificial Sequence
  Synthetic Construct
ggtcgtgcacaacacggccc

SEQ ID NO: 500 - RNA, 20 nt, Artificial Sequence
  Synthetic Construct
tgccattgggcaggccttgg

SEQ ID NO: 501 - RNA, 20 nt, Artificial Sequence
  Synthetic Construct
cacctgcttggccaggagct

SEQ ID NO: 502 - RNA, 20 nt, Artificial Sequence
  Synthetic Construct
cagggagtcctgagtgagga

SEQ ID NO: 503 - RNA, 20 nt, Artificial Sequence
  Synthetic Construct
gggcgtcagctggtagagct

SEQ ID NO: 504 - RNA, 20 nt, Artificial Sequence
  Synthetic Construct
ctgtttatccaaggcaaggg

SEQ ID NO: 505 - RNA, 20 nt, Artificial Sequence
  Synthetic Construct
gaaggcactttgggcctccc

SEQ ID NO: 506 - RNA, 20 nt, Artificial Sequence
  Synthetic Construct
ttctggaggatgattccgga

SEQ ID NO: 507 - RNA, 20 nt, Artificial Sequence
  Synthetic Construct
agtaggaactggttctggcc

SEQ ID NO: 508 - RNA, 20 nt, Artificial Sequence
  Synthetic Construct
aggccaccgttgtgactgga

SEQ ID NO: 509 - RNA, 20 nt, Artificial Sequence
  Synthetic Construct
ggatgacgatgctgctgcct

SEQ ID NO: 510 - RNA, 20 nt, Artificial Sequence
  Synthetic Construct
cgggctgtcgctggagaagc

SEQ ID NO: 511 - RNA, 20 nt, Artificial Sequence
  Synthetic Construct
gtgcacgaccttgctgagcc

SEQ ID NO: 512 - RNA, 20 nt, Artificial Sequence
  Synthetic Construct
ctggggcaggaacatctgca

SEQ ID NO: 513 - RNA, 20 nt, Artificial Sequence
  Synthetic Construct
gtcgccaaaggagctggcct

SEQ ID NO: 514 - RNA, 20 nt, Artificial Sequence
  Synthetic Construct
tcaagatcaagcaggaagcc

SEQ ID NO: 515 - RNA, 20 nt, Artificial Sequence
  Synthetic Construct
atctggaaggtccgaggagg

SEQ ID NO: 516 - RNA, 20 nt, Artificial Sequence
  Synthetic Construct
ctcggccaccttgagggtag

SEQ ID NO: 517 - RNA, 20 nt, Artificial Sequence
  Synthetic Construct
catgaacgtcaggttctgcg

SEQ ID NO: 518 - RNA, 20 nt, Artificial Sequence
  Synthetic Construct
ggatgattccggacggcacc

SEQ ID NO: 519 - RNA, 20 nt, Artificial Sequence
  Synthetic Construct
cctcttggaggctctgctgg

SEQ ID NO: 520 - RNA, 20 nt, Artificial Sequence
  Synthetic Construct
gaactctgtgttcggaggcg

SEQ ID NO: 521 - RNA, 20 nt, Artificial Sequence
  Synthetic Construct
atatttattgagcatggcct

SEQ ID NO: 522 - RNA, 20 nt, Artificial Sequence
  Synthetic Construct
cgactcggacttgcgccgct

SEQ ID NO: 523 - RNA, 20 nt, Artificial Sequence
  Synthetic Construct
ctcacaaaccagaacctggc

SEQ ID NO: 524 - RNA, 20 nt, Artificial Sequence
  Synthetic Construct
tttgtggtagtcactggggg

SEQ ID NO: 525 - RNA, 20 nt, Artificial Sequence
  Synthetic Construct
tcattaccatctccgctgag

SEQ ID NO: 526 - RNA, 20 nt, Artificial Sequence
  Synthetic Construct
gagcatttgcacaaacacca

SEQ ID NO: 527 - RNA, 20 nt, Artificial Sequence
  Synthetic Construct
tcgtccactttggcgggcag

SEQ ID NO: 528 - RNA, 20 nt, Artificial Sequence
  Synthetic Construct
atcctgggctctcctgcgac

SEQ ID NO: 529 - RNA, 20 nt, Artificial Sequence
  Synthetic Construct
gcatctggaagagcgcaggc

SEQ ID NO: 530 - RNA, 20 nt, Artificial Sequence
  Synthetic Construct
ctcgccctggatggtgagcg

SEQ ID NO: 531 - RNA, 20 nt, Artificial Sequence
  Synthetic Construct
ttgagcatggccacggcgct

SEQ ID NO: 532 - RNA, 20 nt, Artificial Sequence
  Synthetic Construct
gggcgtgttgagcgccatga

SEQ ID NO: 533 - RNA, 20 nt, Artificial Sequence
  Synthetic Construct
atccatgcaagaagtcattg

SEQ ID NO: 534 - RNA, 20 nt, Artificial Sequence
  Synthetic Construct
tcggccaccttgagggtagc

SEQ ID NO: 535 - RNA, 20 nt, Artificial Sequence
  Synthetic Construct
ccagctgtttatccaaggca

SEQ ID NO: 536 - RNA, 20 nt, Artificial Sequence
  Synthetic Construct
ctcccagctcctggccaagc

SEQ ID NO: 537 - RNA, 20 nt, Artificial Sequence
  Synthetic Construct
acaagcttggtgggcaggtg

SEQ ID NO: 538 - RNA, 20 nt, Artificial Sequence
  Synthetic Construct
gctgtaccagcgtatgctga

SEQ ID NO: 539 - RNA, 20 nt, Artificial Sequence
  Synthetic Construct
cttggataaacagctggcca

SEQ ID NO: 540 - RNA, 20 nt, Artificial Sequence
  Synthetic Construct
cgtaggtcttgatgggcggg

SEQ ID NO: 541 - RNA, 20 nt, Artificial Sequence
  Synthetic Construct
tccccgccgccatgaacgtc

SEQ ID NO: 542 - RNA, 20 nt, Artificial Sequence
  Synthetic Construct
gagccgatatttattgagca

SEQ ID NO: 543 - RNA, 20 nt, Artificial Sequence
  Synthetic Construct
gaggccaccgttgtgactgg

SEQ ID NO: 544 - RNA, 20 nt, Artificial Sequence
  Synthetic Construct
aagaagtcattgagggcctg

SEQ ID NO: 545 - RNA, 20 nt, Artificial Sequence
  Synthetic Construct
ctcaagatcaagcaggaagc

SEQ ID NO: 546 - RNA, 20 nt, Artificial Sequence
  Synthetic Construct
caggttctggtttgtgagga

SEQ ID NO: 547 - RNA, 20 nt, Artificial Sequence
  Synthetic Construct
gtcaggttctgcggggcctt

SEQ ID NO: 548 - RNA, 20 nt, Artificial Sequence
  Synthetic Construct
caaagatgccaggcagagtt

SEQ ID NO: 549 - RNA, 20 nt, Artificial Sequence
  Synthetic Construct
gcaggctgcaccgtgaggac

SEQ ID NO: 550 - RNA, 20 nt, Artificial Sequence
  Synthetic Construct
tgttgagcgccatgacgggc

SEQ ID NO: 551 - RNA, 20 nt, Artificial Sequence
  Synthetic Construct
gtgcaaatgctccaggaaac

SEQ ID NO: 552 - RNA, 20 nt, Artificial Sequence
  Synthetic Construct
aacctgacgttcatggcggc

SEQ ID NO: 553 - RNA, 20 nt, Artificial Sequence
  Synthetic Construct
cttccagatgcccgtgtcgc

SEQ ID NO: 554 - RNA, 20 nt, Artificial Sequence
  Synthetic Construct
tgctgcctgggaagagggct

SEQ ID NO: 555 - RNA, 20 nt, Artificial Sequence
  Synthetic Construct
acactctgtggccgggaggg

SEQ ID NO: 556 - RNA, 20 nt, Artificial Sequence
  Synthetic Construct
tcggacttgcgccgcttggc

SEQ ID NO: 557 - RNA, 20 nt, Artificial Sequence
  Synthetic Construct
cttgttctggaggatgattc

SEQ ID NO: 558 - RNA, 20 nt, Artificial Sequence
  Synthetic Construct
gcaaaagccttgttctcggc

SEQ ID NO: 559 - RNA, 20 nt, Artificial Sequence
  Synthetic Construct
cgatgagctgtccacctgtg

SEQ ID NO: 560 - RNA, 20 nt, Artificial Sequence
  Synthetic Construct
gctgcccgccaaagtggacg

SEQ ID NO: 561 - RNA, 20 nt, Artificial Sequence
  Synthetic Construct
cccccccagccttgttctgg

SEQ ID NO: 562 - RNA, 20 nt, Artificial Sequence
  Synthetic Construct
gccgaggccaccgttgtgac

SEQ ID NO: 563 - RNA, 20 nt, Artificial Sequence
  Synthetic Construct
cgaccgaatgttcattcagg

SEQ ID NO: 564 - RNA, 20 nt, Artificial Sequence
  Synthetic Construct
cttcctgcagcctgtgggcc

SEQ ID NO: 565 - RNA, 20 nt, Artificial Sequence
  Synthetic Construct
ccgccaggttctggtttgtg

SEQ ID NO: 566 - RNA, 20 nt, Artificial Sequence
  Synthetic Construct
tccagtcacaacggtggcct

SEQ ID NO: 567 - RNA, 20 nt, Artificial Sequence
  Synthetic Construct
gatatttattgagcatggcc

SEQ ID NO: 568 - RNA, 20 nt, Artificial Sequence
  Synthetic Construct
ggctgccattgggcaggcct

SEQ ID NO: 569 - RNA, 20 nt, Artificial Sequence
  Synthetic Construct
tggtgggtcagcacggtagg

SEQ ID NO: 570 - RNA, 20 nt, Artificial Sequence
  Synthetic Construct
ttcctgcagcctgtgggcct

SEQ ID NO: 571 - RNA, 20 nt, Artificial Sequence
  Synthetic Construct
gcgccctggtagacatggta

SEQ ID NO: 572 - RNA, 20 nt, Artificial Sequence
  Synthetic Construct
tgccggaagcttcttgcaca

SEQ ID NO: 573 - RNA, 20 nt, Artificial Sequence
  Synthetic Construct
cctccagcagagcctccaag

SEQ ID NO: 574 - RNA, 20 nt, Artificial Sequence
  Synthetic Construct
gtagtcactgggggagggga

SEQ ID NO: 575 - RNA, 20 nt, Artificial Sequence
  Synthetic Construct
cgtcaggttctgcggggcct

SEQ ID NO: 576 - RNA, 20 nt, Artificial Sequence
  Synthetic Construct
caggttcaggatgctgtcga

SEQ ID NO: 577 - RNA, 20 nt, Artificial Sequence
  Synthetic Construct
tgtcgctggagaagctggcc

SEQ ID NO: 578 - RNA, 20 nt, Artificial Sequence
  Synthetic Construct
ggccagaacgtggtgctgtc

SEQ ID NO: 579 - RNA, 20 nt, Artificial Sequence
  Synthetic Construct
gagctgtccacctgtgtggg

SEQ ID NO: 580 - RNA, 20 nt, Artificial Sequence
  Synthetic Construct
gctccagttcccacccagcc

SEQ ID NO: 581 - RNA, 20 nt, Artificial Sequence
  Synthetic Construct
ggctgggtgggaactggagc

SEQ ID NO: 582 - RNA, 20 nt, Artificial Sequence
  Synthetic Construct
gtagcgaacgtgtccggcgt

SEQ ID NO: 583 - RNA, 20 nt, Artificial Sequence
  Synthetic Construct
gaccggaacgatctcgcgta

SEQ ID NO: 584 - RNA, 20 nt, Artificial Sequence
  Synthetic Construct
ggcagtcgttcggttgatat

SEQ ID NO: 585 - RNA, 20 nt, Artificial Sequence
  Synthetic Construct
gcttgagcacatacgcgaat

SEQ ID NO: 586 - RNA, 20 nt, Artificial Sequence
  Synthetic Construct
gtggtagaataacgtattac

SEQ ID NO: 587 - RNA, 20 nt, Artificial Sequence
  Synthetic Construct
gtcatacatggataaggcta

SEQ ID NO: 588 - RNA, 20 nt, Artificial Sequence
  Synthetic Construct
gatacacgaagcatcactag

SEQ ID NO: 589 - RNA, 20 nt, Artificial Sequence
  Synthetic Construct
gaacgttggcactacttcac

SEQ ID NO: 590 - RNA, 20 nt, Artificial Sequence
  Synthetic Construct
gatccatgtaatgcgttcga

SEQ ID NO: 591 - RNA, 20 nt, Artificial Sequence
  Synthetic Construct
gtcgtgaagtgcattcgatc

SEQ ID NO: 592 - RNA, 20 nt, Artificial Sequence
  Synthetic Construct
gttcgactcgcgtgaccgta

SEQ ID NO: 593 - RNA, 20 nt, Artificial Sequence
  Synthetic Construct
gaatctaccgcagcggttcg

SEQ ID NO: 594 - RNA, 20 nt, Artificial Sequence
  Synthetic Construct
gaagtgacgtcgattcgata

SEQ ID NO: 595 - RNA, 20 nt, Artificial Sequence
  Synthetic Construct
gcggtgtatgacaaccgccg

SEQ ID NO: 596 - RNA, 20 nt, Artificial Sequence
  Synthetic Construct
gtaccgcgcctgaagttcgc

SEQ ID NO: 597 - RNA, 20 nt, Artificial Sequence
  Synthetic Construct
gcagctcgtgtgtcgtactc

SEQ ID NO: 598 - RNA, 20 nt, Artificial Sequence
  Synthetic Construct
gcgccttaagagtactcatc

SEQ ID NO: 599 - RNA, 20 nt, Artificial Sequence
  Synthetic Construct
gagtgtcgtcgttgctccta

SEQ ID NO: 600 - RNA, 20 nt, Artificial Sequence
  Synthetic Construct
gcagctcgacctcaagccgt

SEQ ID NO: 601 - RNA, 20 nt, Artificial Sequence
  Synthetic Construct
gtatcctgacctacgcgctg

SEQ ID NO: 602 - RNA, 20 nt, Artificial Sequence
  Synthetic Construct
gtgtatctcagcacgctaac

SEQ ID NO: 603 - RNA, 20 nt, Artificial Sequence
  Synthetic Construct
gtcgtcatacaacggcaacg

SEQ ID NO: 604 - RNA, 20 nt, Artificial Sequence
  Synthetic Construct
gtcgtgcgcttccggcggta

SEQ ID NO: 605 - RNA, 20 nt, Artificial Sequence
  Synthetic Construct
gcggtcctcagtaagcgcgt

SEQ ID NO: 606 - RNA, 20 nt, Artificial Sequence
  Synthetic Construct
gctctgctgcggaaggattc

SEQ ID NO: 607 - RNA, 20 nt, Artificial Sequence
  Synthetic Construct
gcatggaggagcgtcgcaga

SEQ ID NO: 608 - RNA, 20 nt, Artificial Sequence
  Synthetic Construct
gtagcgcgcgtaggagtggc

SEQ ID NO: 609 - RNA, 20 nt, Artificial Sequence
  Synthetic Construct
gatcacctgcattcgtacac

SEQ ID NO: 610 - RNA, 20 nt, Artificial Sequence
  Synthetic Construct
gcacacctagatatcgaatg

SEQ ID NO: 611 - RNA, 20 nt, Artificial Sequence
  Synthetic Construct
gttgatcaacgcgcttcgcg

SEQ ID NO: 612 - RNA, 20 nt, Artificial Sequence
  Synthetic Construct
gcgtctcactcactccatcg

SEQ ID NO: 613 - RNA, 20 nt, Artificial Sequence
  Synthetic Construct
gccgaccaacgtcagcggta

SEQ ID NO: 614 - RNA, 20 nt, Artificial Sequence
  Synthetic Construct
ggatacggtgcgtcaatcta

SEQ ID NO: 615 - RNA, 20 nt, Artificial Sequence
  Synthetic Construct
gaatccagtggcggcgacaa

SEQ ID NO: 616 - RNA, 20 nt, Artificial Sequence
  Synthetic Construct
gcactgtcagtgcaacgata

SEQ ID NO: 617 - RNA, 20 nt, Artificial Sequence
  Synthetic Construct
gcgatcctcaagtatgctca

SEQ ID NO: 618 - RNA, 20 nt, Artificial Sequence
  Synthetic Construct
gctaatatcgacacggccgc

SEQ ID NO: 619 - RNA, 20 nt, Artificial Sequence
  Synthetic Construct
ggagatgcatcgaagtcgat

SEQ ID NO: 620 - RNA, 20 nt, Artificial Sequence
  Synthetic Construct
ggatgcactccatctcgtct

SEQ ID NO: 621 - RNA, 20 nt, Artificial Sequence
  Synthetic Construct
gtgccgagtaataacgcgag

SEQ ID NO: 622 - RNA, 20 nt, Artificial Sequence
  Synthetic Construct
gagattccgatgtaacgtac

SEQ ID NO: 623 - RNA, 20 nt, Artificial Sequence
  Synthetic Construct
gtcgtcacgagcaggattgc

SEQ ID NO: 624 - RNA, 20 nt, Artificial Sequence
  Synthetic Construct
gcgttagtcacttagctcga

SEQ ID NO: 625 - RNA, 20 nt, Artificial Sequence
  Synthetic Construct
gttcacacggtgtcggatag

SEQ ID NO: 626 - RNA, 20 nt, Artificial Sequence
  Synthetic Construct
ggataggtgaccttagtacg

SEQ ID NO: 627 - RNA, 20 nt, Artificial Sequence
  Synthetic Construct
gtatgagtcaagctaatgcg

SEQ ID NO: 628 - RNA, 20 nt, Artificial Sequence
  Synthetic Construct
gcaactattggaatacgtga

SEQ ID NO: 629 - RNA, 20 nt, Artificial Sequence
  Synthetic Construct
gttaccttcgctcgtctata

SEQ ID NO: 630 - RNA, 20 nt, Artificial Sequence
  Synthetic Construct
gtaccgagcaccacaggccg

SEQ ID NO: 631 - RNA, 20 nt, Artificial Sequence
  Synthetic Construct
gtcagccatcggatagagat

SEQ ID NO: 632 - RNA, 20 nt, Artificial Sequence
  Synthetic Construct
gtacggcactcctagccgct

SEQ ID NO: 633 - RNA, 20 nt, Artificial Sequence
  Synthetic Construct
ggtcctgtcgtatgcttgca

SEQ ID NO: 634 - RNA, 20 nt, Artificial Sequence
  Synthetic Construct
gccgcaatatatgcggtaag

SEQ ID NO: 635 - RNA, 20 nt, Artificial Sequence
  Synthetic Construct
gcgcacgtataatcctgcgt

SEQ ID NO: 636 - RNA, 20 nt, Artificial Sequence
  Synthetic Construct
gtgcacaacacgatccacga

SEQ ID NO: 637 - RNA, 20 nt, Artificial Sequence
  Synthetic Construct
gcacaatgttgacgtaagtg

SEQ ID NO: 638 - RNA, 20 nt, Artificial Sequence
  Synthetic Construct
gtaagatgctgctcaccgtg

SEQ ID NO: 639 - RNA, 20 nt, Artificial Sequence
  Synthetic Construct
gtcggtgatccaacgtatcg

SEQ ID NO: 640 - RNA, 20 nt, Artificial Sequence
  Synthetic Construct
gagctagtaggacgcaagac

SEQ ID NO: 641 - RNA, 20 nt, Artificial Sequence
  Synthetic Construct
gtacgtggaagcttgtggcc

SEQ ID NO: 642 - RNA, 20 nt, Artificial Sequence
  Synthetic Construct
gagaactgccagttctcgat

SEQ ID NO: 643 - RNA, 20 nt, Artificial Sequence
  Synthetic Construct
gccattcggcgcggcacttc

SEQ ID NO: 644 - RNA, 20 nt, Artificial Sequence
  Synthetic Construct
gcacacgaccaatccgcttc

SEQ ID NO: 645 - RNA, 20 nt, Artificial Sequence
  Synthetic Construct
gaggtgatcgattaagtaca

SEQ ID NO: 646 - RNA, 20 nt, Artificial Sequence
  Synthetic Construct
gtcactcgcagacgcctaac

SEQ ID NO: 647 - RNA, 20 nt, Artificial Sequence
  Synthetic Construct
gcgctacggaatcatacgtt

SEQ ID NO: 648 - RNA, 20 nt, Artificial Sequence
  Synthetic Construct
ggtaggacctcacggcgcgc

SEQ ID NO: 649 - RNA, 20 nt, Artificial Sequence
  Synthetic Construct
gaactgcatcttgttgtagt

SEQ ID NO: 650 - RNA, 20 nt, Artificial Sequence
  Synthetic Construct
gatcctgatccggcggcgcg

SEQ ID NO: 651 - RNA, 20 nt, Artificial Sequence
  Synthetic Construct
ggtatgcgcgatcctgagtt

SEQ ID NO: 652 - RNA, 20 nt, Artificial Sequence
  Synthetic Construct
gcggagctagagagcggtca

SEQ ID NO: 653 - RNA, 20 nt, Artificial Sequence
  Synthetic Construct
gaatggcaattacggctgat

SEQ ID NO: 654 - RNA, 20 nt, Artificial Sequence
  Synthetic Construct
gtatggtgagtagtcgcttg

SEQ ID NO: 655 - RNA, 20 nt, Artificial Sequence
  Synthetic Construct
gtgtaattgcgtctagtcgg

SEQ ID NO: 656 - RNA, 20 nt, Artificial Sequence
  Synthetic Construct
ggtcctggcgaggagccttg

SEQ ID NO: 657 - RNA, 20 nt, Artificial Sequence
  Synthetic Construct
gaagataagtcgctgtctcg

SEQ ID NO: 658 - RNA, 20 nt, Artificial Sequence
  Synthetic Construct
gtcggcgttctgttgtgact

SEQ ID NO: 659 - RNA, 20 nt, Artificial Sequence
  Synthetic Construct
gaggcaagccgttaggtgta

SEQ ID NO: 660 - RNA, 20 nt, Artificial Sequence
  Synthetic Construct
gcggatccagatctcattcg

SEQ ID NO: 661 - RNA, 20 nt, Artificial Sequence
  Synthetic Construct
ggaacataggagcacgtagt

SEQ ID NO: 662 - RNA, 20 nt, Artificial Sequence
  Synthetic Construct
gtcatcattatggcgtaagg

SEQ ID NO: 663 - RNA, 20 nt, Artificial Sequence
  Synthetic Construct
gcgactagcgccatgagcgg

SEQ ID NO: 664 - RNA, 20 nt, Artificial Sequence
  Synthetic Construct
ggcgaagttcgacatgacac

SEQ ID NO: 665 - RNA, 20 nt, Artificial Sequence
  Synthetic Construct
gctgtcgtgtggaggctatg

SEQ ID NO: 666 - RNA, 20 nt, Artificial Sequence
  Synthetic Construct
gcggagagcattgacctcat

SEQ ID NO: 667 - RNA, 20 nt, Artificial Sequence
  Synthetic Construct
gactaatggaccaagtcagt

SEQ ID NO: 668 - RNA, 20 nt, Artificial Sequence
  Synthetic Construct
gcggattagaggtaatgcgg

SEQ ID NO: 669 - RNA, 20 nt, Artificial Sequence
  Synthetic Construct
gccgacggcaatcagtacgc

SEQ ID NO: 670 - RNA, 20 nt, Artificial Sequence
  Synthetic Construct
gtaacctctcgagcgataga

SEQ ID NO: 671 - RNA, 20 nt, Artificial Sequence
  Synthetic Construct
gacttgtatgtggcttacgg

SEQ ID NO: 672 - RNA, 20 nt, Artificial Sequence
  Synthetic Construct
gtcactgtggtcgaacatgt

SEQ ID NO: 673 - RNA, 20 nt, Artificial Sequence
  Synthetic Construct
gtactccaatccgcgatgac

SEQ ID NO: 674 - RNA, 20 nt, Artificial Sequence
  Synthetic Construct
gcgttggcacgatgttacgg

SEQ ID NO: 675 - RNA, 20 nt, Artificial Sequence
  Synthetic Construct
gaaccagccggctagtatga

SEQ ID NO: 676 - RNA, 20 nt, Artificial Sequence
  Synthetic Construct
gtatactagctaaccacacg

SEQ ID NO: 677 - RNA, 20 nt, Artificial Sequence
  Synthetic Construct
gaatcggaatagttgattcg

SEQ ID NO: 678 - RNA, 20 nt, Artificial Sequence
  Synthetic Construct
gagcacttgcatgaggcggt

SEQ ID NO: 679 - RNA, 20 nt, Artificial Sequence
  Synthetic Construct
gaacggcgatgaagccagcc

SEQ ID NO: 680 - RNA, 20 nt, Artificial Sequence
  Synthetic Construct
gcaaccgagatgagaggttc

SEQ ID NO: 681 - RNA, 20 nt, Artificial Sequence
  Synthetic Construct
gcaagatcaatatgcgtgat

SEQ ID NO: 682 - RNA, 20 nt, Artificial Sequence
  Synthetic Construct
acggaggctaagcgtcgcaa

SEQ ID NO: 683 - RNA, 20 nt, Artificial Sequence
  Synthetic Construct
cgcttccgcggcccgttcaa

SEQ ID NO: 684 - RNA, 20 nt, Artificial Sequence
  Synthetic Construct
atcgtttccgcttaacggcg

SEQ ID NO: 685 - RNA, 20 nt, Artificial Sequence
  Synthetic Construct
gtaggcgcgccgctctctac

SEQ ID NO: 686 - RNA, 20 nt, Artificial Sequence
  Synthetic Construct
ccatatcggggcgagacatg

SEQ ID NO: 687 - RNA, 20 nt, Artificial Sequence
  Synthetic Construct
tactaacgccgctcctacag

SEQ ID NO: 688 - RNA, 20 nt, Artificial Sequence
  Synthetic Construct
tgaggatcatgtcgagcgcc

SEQ ID NO: 689 - RNA, 20 nt, Artificial Sequence
  Synthetic Construct
gggcccgcataggatatcgc

SEQ ID NO: 690 - RNA, 20 nt, Artificial Sequence
  Synthetic Construct
tagacaaccgcggagaatgc

SEQ ID NO: 691 - RNA, 20 nt, Artificial Sequence
  Synthetic Construct
acgggcggctatcgctgact

SEQ ID NO: 692 - RNA, 20 nt, Artificial Sequence
  Synthetic Construct
cgcggaaattttaccgacga

SEQ ID NO: 693 - RNA, 20 nt, Artificial Sequence
  Synthetic Construct
cttacaatcgtcggtccaat

SEQ ID NO: 694 - RNA, 20 nt, Artificial Sequence
  Synthetic Construct
gcgtgcgtcccgggttaccc

SEQ ID NO: 695 - RNA, 20 nt, Artificial Sequence
  Synthetic Construct
cggagtaacaagcggacgga

SEQ ID NO: 696 - RNA, 20 nt, Artificial Sequence
  Synthetic Construct
cgagtgttatacgcaccgtt

SEQ ID NO: 697 - RNA, 20 nt, Artificial Sequence
  Synthetic Construct
cgactaaccggaaacttttt

SEQ ID NO: 698 - RNA, 20 nt, Artificial Sequence
  Synthetic Construct
caacgggttctcccggctac

SEQ ID NO: 699 - RNA, 20 nt, Artificial Sequence
  Synthetic Construct
caggagtcgccgatacgcgt

SEQ ID NO: 700 - RNA, 20 nt, Artificial Sequence
  Synthetic Construct
ttcacgtcgtctcgcgacca

SEQ ID NO: 701 - RNA, 20 nt, Artificial Sequence
  Synthetic Construct
gtgtcggattccgccgctta

SEQ ID NO: 702 - RNA, 20 nt, Artificial Sequence
  Synthetic Construct
cacgaactcacaccgcgcga

SEQ ID NO: 703 - RNA, 20 nt, Artificial Sequence
  Synthetic Construct
cgctagtacgctcctctata

SEQ ID NO: 704 - RNA, 20 nt, Artificial Sequence
  Synthetic Construct
tcgcgcttgggttatacgct

SEQ ID NO: 705 - RNA, 20 nt, Artificial Sequence
  Synthetic Construct
ctatctcgagtggtaatgcg

SEQ ID NO: 706 - RNA, 20 nt, Artificial Sequence
  Synthetic Construct
aatcgactcgaacttcgtgt

SEQ ID NO: 707 - RNA, 20 nt, Artificial Sequence
  Synthetic Construct
cccgatggactataccgaac

SEQ ID NO: 708 - RNA, 20 nt, Artificial Sequence
  Synthetic Construct
acgttcgagtacgaccagct

SEQ ID NO: 709 - RNA, 20 nt, Artificial Sequence
  Synthetic Construct
cgcgacgactcaacctagtc

SEQ ID NO: 710 - RNA, 20 nt, Artificial Sequence
  Synthetic Construct
ggtcaccgatcgagagctag

SEQ ID NO: 711 - RNA, 20 nt, Artificial Sequence
  Synthetic Construct
ctcaaccgaccgtatggtca

SEQ ID NO: 712 - RNA, 20 nt, Artificial Sequence
  Synthetic Construct
cgtattcgactctcaacgcg

SEQ ID NO: 713 - RNA, 20 nt, Artificial Sequence
  Synthetic Construct
ctagccgcccagatcgagcc

SEQ ID NO: 714 - RNA, 20 nt, Artificial Sequence
  Synthetic Construct
gaatcgaccgacactaatgt

SEQ ID NO: 715 - RNA, 20 nt, Artificial Sequence
  Synthetic Construct
acttcagttcggcgtagtca

SEQ ID NO: 716 - RNA, 20 nt, Artificial Sequence
  Synthetic Construct
gtgcgatgtcgcttcaacgt

SEQ ID NO: 717 - RNA, 20 nt, Artificial Sequence
  Synthetic Construct
cgcctaatttccggatcaat

SEQ ID NO: 718 - RNA, 20 nt, Artificial Sequence
  Synthetic Construct
cgtggccggaaccgtcatag

SEQ ID NO: 719 - RNA, 20 nt, Artificial Sequence
  Synthetic Construct
accctccgaatcgtaacgga

SEQ ID NO: 720 - RNA, 20 nt, Artificial Sequence
  Synthetic Construct
aaacggtacgacagcgtgtg

SEQ ID NO: 721 - RNA, 20 nt, Artificial Sequence
  Synthetic Construct
acatagtcgacggctcgatt

SEQ ID NO: 722 - RNA, 20 nt, Artificial Sequence
  Synthetic Construct
gatggcgcttcagtcgtcgg

SEQ ID NO: 723 - RNA, 20 nt, Artificial Sequence
  Synthetic Construct
ataatccggaaacgctcgac

SEQ ID NO: 724 - RNA, 20 nt, Artificial Sequence
  Synthetic Construct
cgccgggctgacaattaacg

SEQ ID NO: 725 - RNA, 20 nt, Artificial Sequence
  Synthetic Construct
cgtcgccatatgccggtggc

SEQ ID NO: 726 - RNA, 20 nt, Artificial Sequence
  Synthetic Construct
cgggcctataacaccatcga

SEQ ID NO: 727 - RNA, 20 nt, Artificial Sequence
  Synthetic Construct
cgccgttccgagatacttga

SEQ ID NO: 728 - RNA, 20 nt, Artificial Sequence
  Synthetic Construct
cgggacgtcgcgaaaatgta

SEQ ID NO: 729 - RNA, 20 nt, Artificial Sequence
  Synthetic Construct
tcggcatacgggacacacgc

SEQ ID NO: 730 - RNA, 20 nt, Artificial Sequence
  Synthetic Construct
agctccatcgccgcgataat

SEQ ID NO: 731 - RNA, 20 nt, Artificial Sequence
  Synthetic Construct
atcgtatcatcagctagcgc

SEQ ID NO: 732 - RNA, 20 nt, Artificial Sequence
  Synthetic Construct
tcgatcgaggttgcattcgg

SEQ ID NO: 733 - RNA, 20 nt, Artificial Sequence
  Synthetic Construct
ctcgacagttcgtcccgagc

SEQ ID NO: 734 - RNA, 20 nt, Artificial Sequence
  Synthetic Construct
cggtagtattaatcgctgac

SEQ ID NO: 735 - RNA, 20 nt, Artificial Sequence
  Synthetic Construct
tgaacgcgtgtttccttgca

SEQ ID NO: 736 - RNA, 20 nt, Artificial Sequence
  Synthetic Construct
cgacgctaggtaacgtagag

SEQ ID NO: 737 - RNA, 20 nt, Artificial Sequence
  Synthetic Construct
cattgttgagcgggcgcgct

SEQ ID NO: 738 - RNA, 20 nt, Artificial Sequence
  Synthetic Construct
ccgctattgaaaccgcccac

SEQ ID NO: 739 - RNA, 20 nt, Artificial Sequence
  Synthetic Construct
agacacgtcaccggtcaaaa

SEQ ID NO: 740 - RNA, 20 nt, Artificial Sequence
  Synthetic Construct
tttacgatctagcggcgtag

SEQ ID NO: 741 - RNA, 20 nt, Artificial Sequence
  Synthetic Construct
ttcgcacgattgcaccttgg

SEQ ID NO: 742 - RNA, 20 nt, Artificial Sequence
  Synthetic Construct
ggttagagactaggcgcgcg

SEQ ID NO: 743 - RNA, 20 nt, Artificial Sequence
  Synthetic Construct
cctccgtgctaacgcggacg

SEQ ID NO: 744 - RNA, 20 nt, Artificial Sequence
  Synthetic Construct
ttatcgcgtagtgctgacgt

SEQ ID NO: 745 - RNA, 20 nt, Artificial Sequence
  Synthetic Construct
tacgcttgcgtttagcgtcc

SEQ ID NO: 746 - RNA, 20 nt, Artificial Sequence
  Synthetic Construct
cgcggcccacgcgtcatcgc

SEQ ID NO: 747 - RNA, 20 nt, Artificial Sequence
  Synthetic Construct
agctcgccatgtcggttctc

SEQ ID NO: 748 - RNA, 20 nt, Artificial Sequence
  Synthetic Construct
aactagcccgagcagcttcg

SEQ ID NO: 749 - RNA, 20 nt, Artificial Sequence
  Synthetic Construct
cgcaaggtgtcggtaaccct

SEQ ID NO: 750 - RNA, 20 nt, Artificial Sequence
  Synthetic Construct
cttcgacgccatcgtgctca

SEQ ID NO: 751 - RNA, 20 nt, Artificial Sequence
  Synthetic Construct
tcctggataccgcgtggtta

SEQ ID NO: 752 - RNA, 20 nt, Artificial Sequence
  Synthetic Construct
atagccgccgctcattactt

SEQ ID NO: 753 - RNA, 20 nt, Artificial Sequence
  Synthetic Construct
gtcgtccgggattacaaaat

SEQ ID NO: 754 - RNA, 20 nt, Artificial Sequence
  Synthetic Construct
taatgctgcacacgccgaat

SEQ ID NO: 755 - RNA, 20 nt, Artificial Sequence
  Synthetic Construct
tatcgcttccgattagtccg

SEQ ID NO: 756 - RNA, 20 nt, Artificial Sequence
  Synthetic Construct
gtaccataccgcgtaccctt

SEQ ID NO: 757 - RNA, 20 nt, Artificial Sequence
  Synthetic Construct
taagatccgcgggtggcaac

SEQ ID NO: 758 - RNA, 20 nt, Artificial Sequence
  Synthetic Construct
gtagacgtcgtgagcttcac

SEQ ID NO: 759 - RNA, 20 nt, Artificial Sequence
  Synthetic Construct
tcgcggacatagggctctaa

SEQ ID NO: 760 - RNA, 20 nt, Artificial Sequence
  Synthetic Construct
agcgcagatagcgcgtatca

SEQ ID NO: 761 - RNA, 20 nt, Artificial Sequence
  Synthetic Construct
gttcgcttcgtaacgaggaa

SEQ ID NO: 762 - RNA, 20 nt, Artificial Sequence
  Synthetic Construct
gacccccgataacttttgac

SEQ ID NO: 763 - RNA, 20 nt, Artificial Sequence
  Synthetic Construct
acgtccatactgtcggctac

SEQ ID NO: 764 - RNA, 20 nt, Artificial Sequence
  Synthetic Construct
gtaccattgccggctcccta

SEQ ID NO: 765 - RNA, 20 nt, Artificial Sequence
  Synthetic Construct
tggttccgtaggtcggtata

SEQ ID NO: 766 - RNA, 20 nt, Artificial Sequence
  Synthetic Construct
tctggcttgacacgaccgtt

SEQ ID NO: 767 - RNA, 20 nt, Artificial Sequence
  Synthetic Construct
cgctaggtccggtaagtgcg

SEQ ID NO: 768 - RNA, 20 nt, Artificial Sequence
  Synthetic Construct
agcacgtaatgtccgtggat

SEQ ID NO: 769 - RNA, 20 nt, Artificial Sequence
  Synthetic Construct
aaggcgcgcgaatgtggcag

SEQ ID NO: 770 - RNA, 20 nt, Artificial Sequence
  Synthetic Construct
actgcggagcgcccaatatc

SEQ ID NO: 771 - RNA, 20 nt, Artificial Sequence
  Synthetic Construct
cgtcgagtgctcgaactcca

SEQ ID NO: 772 - RNA, 20 nt, Artificial Sequence
  Synthetic Construct
tcgcagcggcgtgggatcgg

SEQ ID NO: 773 - RNA, 20 nt, Artificial Sequence
  Synthetic Construct
atctgtcctaattcggatcg

SEQ ID NO: 774 - RNA, 20 nt, Artificial Sequence
  Synthetic Construct
tgcggcgtaatgcttgaaag

SEQ ID NO: 775 - RNA, 20 nt, Artificial Sequence
  Synthetic Construct
cgaacttaatcccgtggcaa

SEQ ID NO: 776 - RNA, 20 nt, Artificial Sequence
  Synthetic Construct
gccgtgttgctggatacgcc

SEQ ID NO: 777 - RNA, 20 nt, Artificial Sequence
  Synthetic Construct
taccctccggatacggactg

SEQ ID NO: 778 - RNA, 20 nt, Artificial Sequence
  Synthetic Construct
ccgttggactatggcgggtc

SEQ ID NO: 779 - RNA, 20 nt, Artificial Sequence
  Synthetic Construct
gtacggggcgatcatccaca

SEQ ID NO: 780 - RNA, 20 nt, Artificial Sequence
  Synthetic Construct
aagagtagtagacgcccggg

SEQ ID NO: 781 - RNA, 20 nt, Artificial Sequence
  Synthetic Construct
aagagcgaatcgatttcgtg

SEQ ID NO: 782 - RNA, 20 nt, Artificial Sequence
  Synthetic Construct
tcaacaccagtgcctgacgg

SEQ ID NO: 783 - RNA, 20 nt, Artificial Sequence
  Synthetic Construct
aaagtagcttcactctctcg

SEQ ID NO: 784 - RNA, 20 nt, Artificial Sequence
  Synthetic Construct
gagccaaccaatagatgtcc

SEQ ID NO: 785 - RNA, 20 nt, Artificial Sequence
  Synthetic Construct
gcgccgccatgaacctagag

SEQ ID NO: 786 - RNA, 20 nt, Artificial Sequence
  Synthetic Construct
acaaaggttggaacagaacc

SEQ ID NO: 787 - RNA, 20 nt, Artificial Sequence
  Synthetic Construct
ggtgaccgggttattgatgt

SEQ ID NO: 788 - RNA, 20 nt, Artificial Sequence
  Synthetic Construct
ttagtggaggactacagagc

SEQ ID NO: 789 - RNA, 20 nt, Artificial Sequence
  Synthetic Construct
acatatagcccgtaaagctg

SEQ ID NO: 790 - RNA, 20 nt, Artificial Sequence
  Synthetic Construct
cgttggcgatgatctccacg

SEQ ID NO: 791 - RNA, 20 nt, Artificial Sequence
  Synthetic Construct
tggccttttctacctcgcgc

SEQ ID NO: 792 - RNA, 20 nt, Artificial Sequence
  Synthetic Construct
aatggagatactcatctggg

SEQ ID NO: 793 - RNA, 20 nt, Artificial Sequence
  Synthetic Construct
gaagcccgtccagaaagtgt

SEQ ID NO: 794 - RNA, 20 nt, Artificial Sequence
  Synthetic Construct
caatctgaggaactccacga

SEQ ID NO: 795 - RNA, 20 nt, Artificial Sequence
  Synthetic Construct
aggctgcggcgcccacgaga

SEQ ID NO: 796 - RNA, 20 nt, Artificial Sequence
  Synthetic Construct
actttgaccaggccttgcta

SEQ ID NO: 797 - RNA, 20 nt, Artificial Sequence
  Synthetic Construct
accttccataactgccacgc

SEQ ID NO: 798 - RNA, 20 nt, Artificial Sequence
  Synthetic Construct
cgaggcgtacatacccaagg

SEQ ID NO: 799 - RNA, 20 nt, Artificial Sequence
  Synthetic Construct
atggtacggccaaatcaaga

SEQ ID NO: 800 - RNA, 20 nt, Artificial Sequence
  Synthetic Construct
tcttgtaatcccatacgcgt

SEQ ID NO: 801 - RNA, 20 nt, Artificial Sequence
  Synthetic Construct
attcacaggacacagagaat

SEQ ID NO: 802 - RNA, 20 nt, Artificial Sequence
  Synthetic Construct
ccagggctccatcctcaaga

SEQ ID NO: 803 - RNA, 20 nt, Artificial Sequence
  Synthetic Construct
tgagctgcaccaaagagacg

SEQ ID NO: 804 - RNA, 20 nt, Artificial Sequence
  Synthetic Construct
atgtctgcagatgtacccct

SEQ ID NO: 805 - RNA, 20 nt, Artificial Sequence
  Synthetic Construct
cgaagataacgcggatacct

SEQ ID NO: 806 - RNA, 20 nt, Artificial Sequence
  Synthetic Construct
gctgcaggccgagtacaccg

SEQ ID NO: 807 - RNA, 20 nt, Artificial Sequence
  Synthetic Construct
acaagtgggaggcttacctg

SEQ ID NO: 808 - RNA, 20 nt, Artificial Sequence
  Synthetic Construct
gcagcgtacagggatgatca

SEQ ID NO: 809 - RNA, 20 nt, Artificial Sequence
  Synthetic Construct
gcagtagcgcttcaggccca

SEQ ID NO: 810 - RNA, 20 nt, Artificial Sequence
  Synthetic Construct
caaatggtggggtaacagaa

SEQ ID NO: 811 - RNA, 20 nt, Artificial Sequence
  Synthetic Construct
gaaaggaactggctaccgtt

SEQ ID NO: 812 - RNA, 20 nt, Artificial Sequence
  Synthetic Construct
agggcttccgttacaagatg

SEQ ID NO: 813 - RNA, 20 nt, Artificial Sequence
  Synthetic Construct
gaacaagcaacacctaaaag

SEQ ID NO: 814 - RNA, 20 nt, Artificial Sequence
  Synthetic Construct
tgaggagaaggaacggctca

SEQ ID NO: 815 - RNA, 20 nt, Artificial Sequence
  Synthetic Construct
ggaagaatgcagagtataag

SEQ ID NO: 816 - RNA, 20 nt, Artificial Sequence
  Synthetic Construct
ggaatttgaggaactcctga

SEQ ID NO: 817 - RNA, 20 nt, Artificial Sequence
  Synthetic Construct
gctcaccggccatccaggaa

SEQ ID NO: 818 - RNA, 20 nt, Artificial Sequence
  Synthetic Construct
actggccaggaacgatgcga

SEQ ID NO: 819 - RNA, 20 nt, Artificial Sequence
  Synthetic Construct
gcagctccaagatcttccca

SEQ ID NO: 820 - RNA, 20 nt, Artificial Sequence
  Synthetic Construct
gaatgagtacacagaacgga

SEQ ID NO: 821 - RNA, 20 nt, Artificial Sequence
  Synthetic Construct
ggagcaggacaaggtcgggg